(19) United States
(12) Patent Application Publication
(10) Pub. No.: US 2026/0191470 A1
(43) Pub. Date: Jul. 9, 2026

(54) Oximeter Method with Durable Unit Reuse and Sheath Disposal

(71) Applicant: ViOptix, Inc., Newark, CA (US)

(72) Inventors: Shaheen Jeeawoody, Hayward, CA (US); Jordan A. Sweer, Laurel, MD (US); Alex Michael Margiott, Dunbarton, NH (US); Steven J. Kuhn, Santa Clara, CA (US); Arik Golden Singh, Palo Alto, CA (US); Robert John de Saint Phalle, Marblehead, MA (US); Cameran Casale, Berkeley, CA (US); Timothy Lee Sauder, San Francisco, CA (US); Peter Callas, Castro Valley, CA (US); Timothy Buskard, Livermore, CA (US); Jeffrey Perkins, Lexington, MA (US); Nicholas L. Mann, Livermore, CA (US); Hailey Alison Gryka, Los Gatos, CA (US); Maw-Lin Kuo, Fremont, CA (US); Lilit Karayan, Sunnyvale, CA (US); Mark Lonsinger, San Jose, CA (US); Scott E. Coleridge, New York, NY (US)

(21) Appl. No.: 19/444,118

(22) Filed: Jan. 8, 2026

Related U.S. Application Data
(60) Provisional application No. 63/743,228, filed on Jan. 8, 2025.
(60) Provisional application No. 63/895,914, filed on Oct. 8, 2025.

(51) Int. Cl. A61B 1/313; A61B 1/00; A61B 1/07; A61B 5/00; A61B 5/1455; A61B 5/1459

(52) U.S. Cl. CPC A61B 5/6847; A61B 5/14551; A61B 5/1459; A61B 2562/146; A61B 2562/166; A61M 2025/0681

(57) ABSTRACT

A medical device includes a replaceable or disposable sheath and laparoscopic sensor, which is at an end or tip of a laparoscopic tube. A durable or processing unit fits into and is enclosed by the disposable sheath, such that the processing unit connects to and operates in conjunction with a light engine of the sheath, to form a laparoscopic oximeter. This oximeter can measure oxygen saturation laparoscopically. The sheath can be disposed of after a single use. The processing unit can be removed from a used sheath and then used multiple additional times with clean, unused sheaths.

### CROSS-REFERENCE TO RELATED APPLICATIONS

This application claims the benefit of U.S. patent applications 63/895,914, filed Oct. 8, 2025, and 63/743,228, filed Jan. 8, 2025. These applications are incorporated by reference along with all references cited in these applications and this application.

### BACKGROUND OF THE INVENTION

The invention generally relates to optical systems that monitor oxygen levels in tissue. More specifically, the invention relates to optical sensors, such as laparoscopic oximeters, that include source structures and detector structures in the laparoscopic oximeters.

Oximeters are medical devices used to measure the level of oxygen in the vessels, tissues, and organs of humans and other living things for various purposes. For example, oximeters are used for medical and diagnostic purposes in hospitals and other medical facilities (e.g., surgery, patient monitoring, or ambulance or other mobile monitoring for, e.g., hypoxia); sports and athletics purposes at a sports arena (e.g., professional athlete monitoring); personal or at-home monitoring of individuals (e.g., general health monitoring, or personal training for a marathon); and veterinary purposes (e.g., animal monitoring).

Pulse oximeters and tissue oximeters are two types of oximeters that operate on different principles. A pulse oximeter uses a patient's pulse to make measurements. A pulse oximeter typically measures the absorbance of light due to pulsing arterial blood. In contrast, a tissue oximeter does not need a pulse in order to function and can be used to make oxygen saturation measurements of tissue that has been disconnected from a blood supply (tissue flap) or of tissue, such as internal organs, that are connected to a blood supply.

Human tissue includes a variety of light-absorbing molecules referred to as chromophores. Such chromophores in the cells of tissue of a body include oxygenated hemoglobin, deoxygenated hemoglobin, water, lipid, and cytochrome. Oxygenated hemoglobin and deoxygenated hemoglobin are the most dominant chromophores in cells for much of the visible and near-infrared spectral range. Light absorption differs significantly for oxygenated and deoxygenated hemoglobins at certain wavelengths of light. Tissue oximeters can measure oxygen levels in human tissue by exploiting these light-absorption differences.

Despite the success of existing oximeters, there is a continuing desire to improve oximeters, for example, by improving form factor; improving measurement accuracy; reducing measurement time; lowering cost; reducing size, reducing or distributing weight, or reducing form factor, such as for portability; reducing power consumption; and for other reasons, and any combination of these improvements.

In particular, there is a continuing desire to determine and assess a patient's localized tissue oxygenation. A determination and assessment of localized tissue oxygenation is useful in many medical settings, such as in clinical settings, during surgery and recovery, and where it may be suspected that the patient's tissue oxygenation state is unstable. For example, during surgery, oximeters should be able to quickly deliver accurate oxygen saturation measurements for localized patient tissue. While existing oximeters have been sufficient for tissue monitoring where a localized tissue measurement is not critical and trending data alone is sufficient, accuracy is, however, important during surgery in which spot-checking can be used to determine whether tissue might remain viable, such as for transplant, or whether a portion of a piece of tissue needs to be removed because the portion might be suspect for becoming necrotic.

Further, since many surgeries are moving toward less invasive techniques, like laparoscopy, there is a need for improved tissue laparoscopic oximeters and methods of making measurements using these oximeters.

### BRIEF SUMMARY OF THE INVENTION

A medical device includes a replaceable or disposable sheath and laparoscopic sensor, which is at an end or tip of a laparoscopic tube. A durable or processing unit fits into and is enclosed by the disposable sheath, such that the processing unit connects to and operates in conjunction with a light engine of the sheath, to form a laparoscopic oximeter. This oximeter can measure oxygen saturation laparoscopically. The sheath can be disposed of after a single use. The processing unit can be removed from a used sheath and then used multiple additional times with clean, unused sheaths.

A laparoscopic medical device includes an oximeter sensor at its tip, which allows making of oxygen saturation measurements laparoscopically. The laparoscopic medical device includes a durable unit and a measurement unit that includes laparoscopic tube, a sheath, and a lid hinge coupled to the sheath. The lid can be rotated with respect to the sheath to form a sealed inner space in the lid and sheath. When the durable unit is in the sheath, the lid and sheath can be closed so that the durable unit is sealed in the sealed inner space where contaminants cannot reach the durable unit. The durable unit detachably connects with the lid of the measurement unit so that the durable unit can be connected and disconnected from the measurement unit for numerous for reuses of the durable unit. The measurement unit can be disposed of and replaced for different patient surgeries. The lid is integrated with the tube. The sheath and lid into which the durable unit can be sealed provides a sterile space for the unit so that the unit can be used for multiple patient surgeries. The durable unit's reusability facilitates cost savings and ecological conservation since the unit includes costly circuitry.

All public published content by ViOptix, Inc. on or before the filing date of this patent application is incorporated by reference along with all other cited references in this application. This published content includes Web site pages, user guides and manuals, white papers, and other on-line and paper publications and documentation. This application also incorporates by reference U.S. patent applications 63/618,864 and Ser. No. 19/014,122, filed Jan. 8, 2024, and Ser. Nos. 19/012,790, 19/012,792, 19/012,826, and 19/012,833, filed Jan. 7, 2025.

Other objects, features, and advantages of the present invention will become apparent upon consideration of the following detailed description and the accompanying drawings, in which like reference designations represent like features throughout the figures.

### BRIEF DESCRIPTION OF THE DRAWINGS

FIG. 1 shows an image of a laparoscopic oximeter, in an implementation.

FIG. 2 is a diagram of a computing environment to which various techniques described in this application may be applied.

FIG. 3 shows a computer system, in an implementation.

FIG. 4 shows a block diagram of the computer system, in an implementation.

FIG. 5 shows an example client system that can include a smartphone in which some of the various techniques described in this application may be implemented.

FIG. 6 shows an example client system that can include a tablet computing device in which some of the various techniques described in this application may be implemented.

FIG. 7 shows a simplified diagram of a client system in which some of the various techniques described in this application may be implemented.

FIGS. 8A-8K are diagrams of laparoscopic oximeters, in various implementations.

FIG. 9A shows an end view of the sensor head of the laparoscopic oximeter, in an implementation.

FIG. 9B shows an end view of the sensor head, in an implementation.

FIG. 10A shows the durable unit and measurement unit, in an implementation.

FIGS. 10B-10C show front and back views of the oximeter device, in an implementation.

FIG. 10D is a transparent view of the lid, in an implementation.

FIG. 10E shows a view of the sheath in an open configuration and shows interior views of the lid and sheath of the housing, in an implementation.

FIG. 10F shows a view of the sheath in a closed configuration.

FIG. 11A shows the durable unit, the laparoscopic tube, and the sheath, in an implementation where the lid is open and the durable unit is oriented for insertion into the housing portion of the sheath.

FIG. 11B shows the durable unit located in the housing portion of the sheath, in an implementation where the lid is open.

FIG. 11C shows the durable unit located in the sheath 35, in an implementation where the sheath's lid is closed onto the sheath's housing.

FIG. 12 shows an end view of the top of the durable unit.

FIG. 13 shows an end view of the lid connected to the laparoscopic tube.

FIG. 14A shows a view of the durable unit with the battery cover removed from the housing of the durable unit.

FIG. 14B shows an exploded view of the durable unit, in an implementation.

FIG. 14C shows a transparent view of the durable unit, in an implementation.

FIG. 14D shows a front view of the durable unit, in an implementation.

FIG. 14E shows a side view of the durable unit, in an implementation.

FIG. 15A shows a perspective view of the shell of the lid.

FIG. 15B shows a view of the durable unit located in the closed sheath.

FIG. 16A shows a transparent view of the lid, in an implementation.

FIG. 16B shows an exploded view of components housed in the interior space of the lid, in an implementation.

FIGS. 16C-16D show back views of the guide blocks, in an implementation.

FIGS. 16F-16G show views of the PCB of the lid, in an implementation.

FIG. 16H shows a top view of the baffle and the tops of fasteners that connect the guide blocks and to the PCB.

FIG. 16I shows a view of the bottom surface of the PCB, in an implementation.

FIG. 16J shows a side view of the PCB and various elements that are connected to the PCB, in an implementation.

FIGS. 16K-16M show views of a fiber guard, in an implementation.

FIGS. 16N-16P show locations where an adhesive is applied to the housing of the lid and to the housing of the laparoscopic tube.

FIG. 17A shows a transparent view of the lid, in an implementation.

FIG. 17B shows the PCB with the light engine and photodetectors mounted on the PCB.

FIG. 18A shows an end portion of the housing of the laparoscopic tube and the sensor head, in an implementation.

FIG. 18B shows a transparent view of the housing, in an implementation where the laparoscopic oximeter includes 2 source waveguides and 4 detector waveguides.

FIGS. 18C-18D show the source and detector waveguides being inserted into the housing 217 of the laparoscopic tube for assembly.

FIG. 18E shows a transparent view of the housing, in an implementation where the laparoscopic oximeter includes 2 source waveguides and 8 detector waveguides.

FIG. 19 shows an example use of the laparoscopic oximeter in an operating room with a patient during an operation.

FIG. 20 shows an example use of the laparoscopic oximeter in an operating room with a patient, in an implementation.

FIG. 21 shows a process for placing the durable unit into the sheath for a surgery, in an implementation.

FIG. 22 shows the laparoscopic oximeter located in a sheath, in an implementation.

FIG. 23 shows the laparoscopic oximeter located in a sheath, in an implementation.

FIGS. 24-25 show a laparoscopic oximeter, in an implementation.

FIGS. 26-27 show a laparoscopic oximeter, in an implementation.

FIGS. 28-29 show a laparoscopic oximeter, in an implementation.

### DETAILED DESCRIPTION OF THE INVENTION

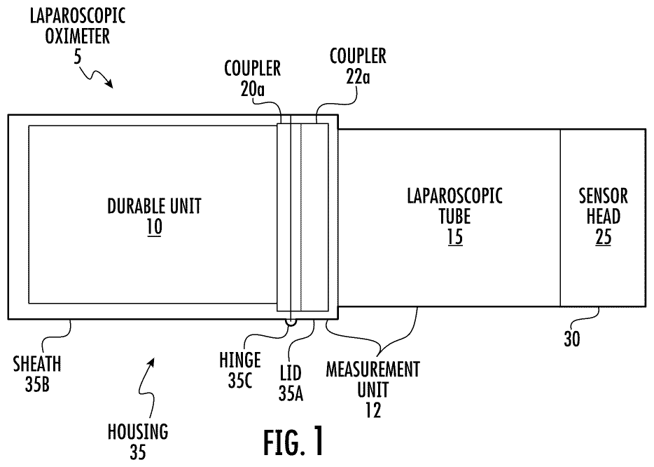

FIG. 1 shows an image of an oximeter device 5. The oximeter device may also be referred to as an oximeter, oximeter apparatus, oximeter probe, or another term. The oximeter device is configured to make tissue oximetry measurements of patient tissue. The oximeter device can be used for invasive procedures, minimally invasive procedures, or noninvasive or not invasive procedures.

In an implementation, the oximeter device includes an elongated tube 15 for laparoscopic use. The oximeter device further includes a housing 35 that includes a lower portion 35a and an upper portion 35b that are sometimes referred to as a lid and a sheath. The lower portion is connected to the upper portion by a hinge 35c. The upper and lower portions can rotate relative to each other via the hinge.

The lower portion includes a first opening at a distal end and a second opening at a proximal end. The upper portion includes a third opening at a distal end and a closed portion at a proximal end. And by rotation of the hinge, the third opening can mate with the second opening to form a sealed enclosed space that is within the upper and lower portions.

The elongated tube is connected to the first opening at the proximal end of the lower portion. The connected elongated tube 15 and housing 35 form a measurement unit or probe 12. And a sensor head 25 or sensor probe is connected to a distal end 30 or tip of the elongated tube. In an implementation where the oximeter device is configured for invasive or minimally invasive procedures, the elongated tube is a laparoscopic tube.

Further, the oximeter device includes a processing unit 10 or portion. The processing unit is sometimes referred to as a durable unit because the unit is reusable. The durable unit includes an enclosure having an interior space. An electrical connector 20a is formed at a distal end of the enclosure. A printed circuit board includes electrical circuitry, where the printed circuit board is enclosed within the interior space and is connected to the electrical connector. A battery compartment is formed in the enclosure. The battery compartment is covered by a battery cover that forms an exterior surface of the enclosure. A display is connected to the enclosure and the printed circuit board.

For operation of the oximeter device, the durable unit is inserted into the lower portion 35b of the housing 35. The durable unit is then sealed in the enclosed space formed by the upper and lower portions of housing 35 when the second and third openings of the upper and lower portions are mated together. The lower portion 35a includes an electrical connector 22a. The electrical connector 22a of the lower portion connects with the electrical connector 20a of the durable unit when the durable unit is sealed in the enclosed space of the housing when the upper and lower portions are closed.

The lower portion includes a light engine that the electrical circuitry of the durable unit connects to. The light engine is located on a printed circuit board with the electrical connector 22a, light emitters, and photodetectors. The light emitters and photodetectors are connected by optical conductors through the elongated tube to the sensor head at the distal end of the elongated tube. In an implementation, the durable unit does not include the light emitters, such as light emitting diodes or laser diodes. In an implementation, the durable unit does not include photodetectors.

The light engine can include a circuit that drives the light emitters to emit light. The light engine can include an analog front-end (AFE) circuit that is connected to the processor through electrical connectors 20a and 22a when the connectors are connected. The processor can control the AFE circuit. The AFE can control the time-multiplexing of electrical signal generation for electrical signal for light generation by the LEDs and for controlling detection by the photodetector for oximeter measurements. In an implementation, the AFE is coupled to the LEDs via one or more multiplexer circuits. In an implementation, the AFE is coupled to the photodetectors via one or more multiplexer circuits. The multiplexer circuits can be the same circuit or different circuits. In another implementation, the AFE is not coupled to the LEDs and photodetectors through a multiplexer or multiplexors but is coupled to the LEDs and photodetectors through fan-out connections.

The light emitters can include two or more light emitters where each light emitter transmits light into a source optical waveguide. Each light emitter can include one or more light emitting diodes (LEDs), one or more laser diodes, or other types of light emitter.

In an implementation, each light emitter includes two or more LEDs, such as three LEDs. The LEDs can be formed on a single circuit that is mounted on PCB 70. Each LED can emit two or more wavelengths of light, such as wavelengths at 760, 810, and 840 nanometers. Other wavelengths can be used. Additional wavelengths can also be used. The wavelengths can be chosen to substantially not interact with surgical dyes that are applied to tissue being measured. For example, the reflectance and transmission of emitted light may not be substantially changed if the light strikes tissue without or with surgical dye, such as indocyanine green or methylene blue.

The depth of tissue for which oximetry measurements are taken can be varied based, for example, on the particular source-detector pair used by the oximeter to collect oximetry data. For example, for a relatively shallow tissue depth measurement (first tissue depth), the source and detector having the shortest separation may be used for collecting oximetry information. For example, for a next deeper tissue depth measurement (second tissue depth), the source and detector having the a second shortest separation may be used for collecting oximetry information, such that the first tissue depth is less than the second tissue depth. For example, for a next deeper tissue depth measurement (third tissue depth), the source and detector having the a third shortest separation may be used for collecting oximetry information, such that the first tissue depth is less than the second tissue depth and the second tissue depth is less than the third tissue depth. Measurement for further tissue depth may be made using sources and detector having larger separations. Oximetry information for a two different tissue depth may be used to generate oximetry information for tissue for the first and second tissue depths, such as by averaging measurements or weighted averaging of measurements.

Lid 35a includes one or more thermistors (not shown), which are used in a temperature correction step of the algorithm. The durable unit also has EEPROM memory 222 to store calibration coefficients.

The durable unit 10 can be reused multiple times, whereas the measurement unit 12 (e.g., sheath, lid, and elongated tube) are typically disposed of after a single use. The durable unit can be reused more than 100 times, such as up to 500 times or more. The disposable measurement unit facilitates ease in maintaining sterility of a procedure, without the time taken for cleaning or sterilizing the previously used unit (although sterilization of the measurement unit after use with a patient may also be possible).

The durable unit is completely sealed in the lid and sheath from the environment for use, and consequently, may not be cleaned for reuse. Alternatively, the durable unit can be cleaned, sanitized, or disinfected for reuse. For example, a durable unit can be cleaned by wiping down the durable unit with a cleaning agent, a sanitizing agent, a disinfecting agent, or any combination of these agents prior to reuse. A durable unit can be cleaned by lightly spraying the durable unit with a cleaning agent, a sanitizing agent, a disinfecting agent, or any combination of these agents prior to reuse. The spray may remain on the durable unit for a period of time, wiped across the durable unit after being sprayed, or both prior to reuse. The durable unit can be lightly exposed to a gaseous sanitizing agent, a disinfecting agent, or both prior to reuse. The durable unit can be exposed to radiation, such as ultraviolet radiation to sanitize, disinfect, or both prior to reuse. Agent for cleaning, sterilizing, or disinfecting can include ethylene oxide (EtO), alcohols (such as Isopropyl alcohol of about a 60%-80% concentration), bleach and other chlorine compounds, hydrogen peroxide, quaternary ammonium compounds, glutaraldehyde, ortho-phthalaldehyde (OPA), peracetic acid, benzyl-C12-18-alkyldimethyl ammonium chlorides, diemthyl chlorides, or other agents.

Thus, the durable unit can be reused multiple times prior to disposal, which lowers the cost of using the oximeter device. Disposal of durable unit 10, measurement unit 12, or both can include recycling or partial recycling.

In an implementation, the measurement unit (e.g., lid, sheath, and elongated tube) is intended to be a disposable unit that is to be disposed of after use with a single patient for a single procedure. In an implementations, the measurement unit can be can be cleaned, sanitized, disinfected, or a combination of these processes for reuse. The measurement unit can be treated via wiping, spraying, gas exposure, or radiation exposure as described immediately above with respect to the durable unit. The measurement unit can be sterilized during manufacture and shipped sterile for use. The measurement unit can be cleaned, sanitized, or disinfected after use so that the measurement can be reused, such as for noninvasive use.

In an implementation, the oximeter device 5 is fully self-contained and does not need to be connected to another device to be fully operational. That is, the oximeter device 5 does not need to be wire-connected or wirelessly connected to another device to operate to make oximetry measurements, store information for the oximetry measurements, display information for the oximetry measurements, generate measurement averages, calibrate oximetry measurements, or perform other functions, or any combination of these functions. Thus, the oximeter device can be used after removal from packaging the without connection to another device, which provides for relatively fast usage from unpacking to use.

In an implementation, the oximeter device 5 does connect to other devices, such as one or more other medical devices, a server system, a computer system (e.g., a laptop computer or a desktop computer), a mobile device (e.g., a smartphone or a tablet computer), a display, these devices or systems, or other devices or systems. The oximeter device can be wire connected or wirelessly connected to these other devices.

In an implementation, when the oximeter device is a laparoscopic oximeter, the oximeter is adapted for intraoperative use in a patient. The elongated housing of the laparoscopic tube of the oximeter can be introduced into the abdominal cavity of the patient through a cannula or other access port, the throat of a patient, or other parts of a patient's body. For example, an outer surface of the elongated housing of the laparoscopic tube can be smooth so that the elongated housing of the laparoscopic tube can slide through the cannula smoothly, can rotate within the cannula smoothly, and can slide into contact and past patient tissue smoothly and without abrading the tissue. The laparoscopic oximeter can be used for invasive procedures, minimally invasive procedures, or non-invasive or not invasive procedures. The laparoscopic oximeter can be used for making oximetry measurements for attached tissue or detached tissue, such as tissue being transplanted that is attached or not attached to a blood supply, such as colon being transplanted or other tissue, sometimes including a tissue flap. The laparoscopic oximeter can be used to make oximetry measurements for a variety of tissues (e.g., internal tissues) to determine various oximetry information for the tissues, such as oxygen saturation. The tissue under test can include intestinal tissue, such as the large intestine, the small intestine, tissue that supports these tissues, such as the mesentery tissue, muscle, the liver, kidneys, stomach, esophagus, or other tissues.

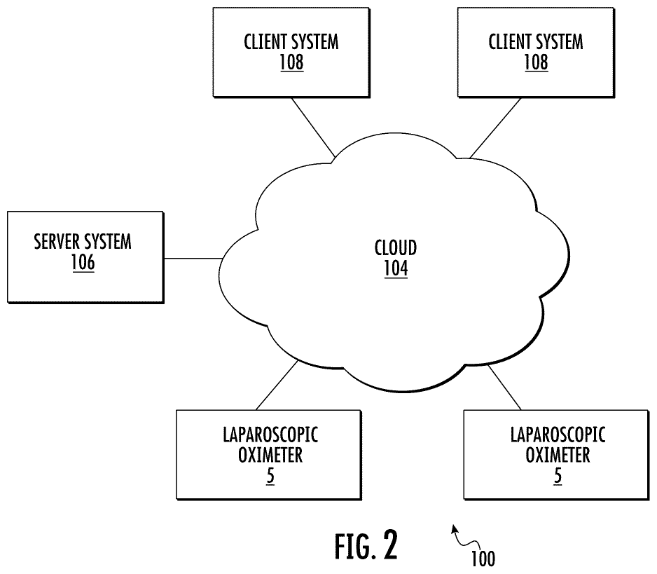

FIG. 2 is a diagram of a computing environment to which various techniques described in this application may be applied. More specifically, FIG. 2 shows a computing environment 100 where a plurality of client systems 108 may be connected with one or more laparoscopic oximeters 5 via a cloud computing environment or a network 104 to provide various oximetry features, oximetry functions, oximetry tasks, oximetry calculations, and other functions. Computing environment 100 may also provide for one or more server systems 108 to communicate with one or more laparoscopic oximeters 5, one or more client systems 108, or both via a cloud computing environment or a network 104 to provide various oximetry features, oximetry functions, oximetry tasks, oximetry calculations, and other functions.

Each client system 108 includes one or more types of computing devices, such as a mobile device (e.g., a smartphone or tablet computer), a laptop computer, a desktop computer, another medical device, another device in a medical care facility, or any combination of these devices or systems, in an implementation. Laparoscopic oximeter 5 can be a medical device system or a patient monitoring system for monitoring oxygen saturation of patient tissue to further determine the viability of the patient tissue. Cloud network 104 can include one or more of a private cloud, a public cloud, a hybrid cloud, the Internet, an intranet, a mesh network, other types of networks, or any combination of these systems.

In an implementation where the client system is a mobile device, such as a smartphone or tablet computer having cellular communication circuits, cloud network 104 is a mobile phone network (i.e., a cellular network) or can include a mobile phone network. The mobile phone network can be a network provided by Verizon Communications Inc., AT&T Inc., T-Mobile US, Inc. T-Mobile in the US, Reliance Jio Infocomm Limited, Bharti Airtel Limited, Vodafone Idea Ltd India, a government provider, or other provider. In the implementation where the client system is a mobile device, the mobile device can have an application that allows for monitoring and displaying oximetry information (e.g., oxygen saturation) generated by the laparoscopic oximetry. The mobile device can be located at a variety of locations, such as the hospital where the laparoscopic oximeter is being used (e.g., at a doctor's office separate from an operating room where the oximeter is being used), at a home (e.g., a doctor's home), in a vehicle, or other location.

The cloud computing environment or network 104 provides for communication by a laparoscopic oximeter 5 or client system 108 with one or more server systems 106, where the server system can include one or more server computers, one or more virtual machines, one or more executable containers, or other systems, in various implementations. A laparoscopic oximeter 5 may be connected to the cloud through a hardwired connection, a wireless connection, or through a combination of both hardwired and wireless transmissions to facilitate various functions described (e.g., monitoring the viability of biological tissue, intestinal ischemia, bowel ischemia, peripheral vascular disease, and others, measurement of changes in the concentrations of oxygenated hemoglobin, deoxygenated hemoglobin, and other tissue components.

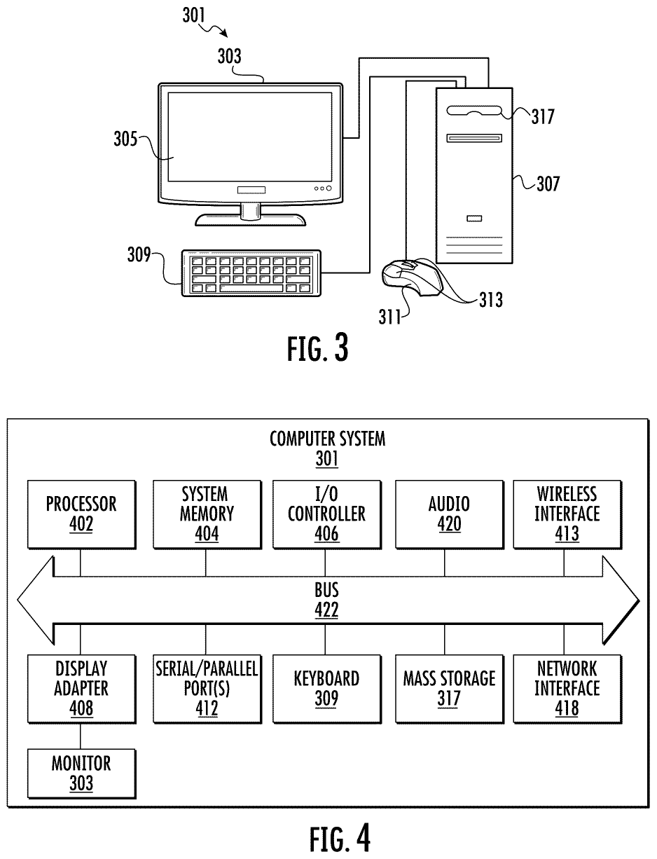

FIG. 3 shows a computer system 301, in an implementation. The computer system can be server system 106, client system 108, or another medical device system, in various implementations. In one implementation, computer system 301 includes a monitor 303 having a display screen 305, an enclosure 307 (may also be referred to as a system unit, cabinet, or case), a keyboard 309, a mouse 311 having one or more mouse buttons 313, scroll wheels, track balls, or other buttons, another type of pointing device, another human input device, or any combination of these devices. For example, where the computer system 301 is a client system 108, the computer system includes each of the devices shown in FIG. 3 and described. Where the computer system 301 is a server system 106, the computer system may not include the monitor, the keyboard, and the mouse. In this implementation, the computer system 301 may be a server system located in a server farm that is located at a data center facility. The server system can work together with other server systems located at the data center facility to support shared workloads such as hosting websites, hosting databases, providing various calculations (e.g., oximetry calculations), providing for various cloud services, other functions, or any combination of these functions. These facilities provide essential infrastructure like reliable power, cooling, and network systems to ensure high performance, scalability, and continuous availability for various digital applications.

It should be understood that the computing systems described are not limited to any computing device in a specific form factor (e.g., desktop computer form factor), but can include all types of computing devices in various form factors. A user can interact with any computing device, including smartphones, personal computers, laptops, electronic tablet devices, global positioning system (GPS) receivers, portable media players, personal digital assistants (PDAs), other network access devices, and other processing devices capable of receiving or transmitting data, where the user can be a human user or a computer user.

For example, in a specific implementation, the client system 108 can be a smartphone or tablet device, such as the Apple iPhone (e.g., Apple iphone 16), Apple iPad (e.g., Apple iPad, Apple ipad Air, Apple ipad Pro, or Apple ipad mini), Samsung Galaxy product (e.g., Galaxy S series product or Galaxy Note series product), Google Nexus and Pixel devices (e.g., Google Pixel 7, Google Pixel 8, Google Pixel 9, or Google Pixel 10), and Microsoft devices (e.g., Microsoft Surface tablet). Typically, a smartphone includes a telephony portion (and associated radio frequency (RF) communication modules) and a computer portion, which are accessible to a human user via a touch screen display.

There is nonvolatile memory configured to store data of the telephone portion (e.g., contacts and phone numbers), and the computer portion operates various applications (e.g., application programs including a browser, pictures, games, videos, and music). The smartphone typically includes a camera (e.g., front-facing camera, rear-facing camera, or both) for taking pictures and video. For example, a smartphone or tablet computer can be used to take live video that can be streamed to one or more other devices.

Enclosure 307 houses familiar computer components, such as a processor, memory, Fm storage devices 317, and the like. Mass storage devices 317 may include mass disk drives, floppy disks, magnetic disks, optical disks, magneto-optical disks, fixed disks, hard disks, CD-ROMs, recordable CDs, DVDs, recordable DVDs (e.g., DVD-R, DVD+R, DVD-RW, DVD+RW, HD-DVD, or Blu-ray Disc), flash and other nonvolatile solid-state storage (e.g., USB flash drive or solid state drive (SSD)), battery-backed-up volatile memory, tape storage, reader, and other similar media, and combinations of these. In an implementation where the enclosure is a server system, mass storage devices may be housed outside of enclosure 307. In the server system implementation, the server may store and operate a server operating system.

A computer-implemented or computer-executable version or computer program product of the described implementations may be embodied using, stored on, or associated with a computer-readable medium. A computer-readable medium may include any medium that participates in providing instructions to one or more processors for execution. Such a medium may take many forms, including, but not limited to, nonvolatile memory, volatile memory, and transmission media. Nonvolatile memory includes, for example, flash memory, optical disk memory, or magnetic disk memory. Volatile memory includes static or dynamic memory, such as cache memory or RAM. Transmission media includes coaxial cables, copper wire, fiber optic lines, and wires arranged in a bus. Transmission media can also take the form of electromagnetic, radio frequency, acoustic, or light waves, such as those generated during radio wave and infrared data communications.

For example, a binary machine-executable version of the software, in an implementation, may be stored or reside in RAM, cache memory, or on a mass storage device 317. The source code of the software of the implementation may also be stored or reside on mass storage device 317 (e.g., hard disk, magnetic disk, tape, or CD-ROM). As a further example, the code of the described implementations may be transmitted via wires, radio waves, or through a network such as the Internet.

FIG. 4 shows a block diagram of computer system 301 used to execute the computer code, in an implementation. As in FIG. 3, computer system 301 includes monitor 303, keyboard 309, and mass storage devices 317. Computer system 301 further includes subsystems such as central processor 402, system memory 404 (e.g., volatile and nonvolatile memory), input/output (I/O) controller 406, display adapter 408, serial or universal serial bus (USB) port 412, wireless interface (e.g., RF interface, such as a Bluetooth interface), network interface 418, and speaker 420. The laparoscopic oximeter may also be used with computer systems with additional or fewer subsystems. For example, a computer system could include more than one processor 402 (i.e., a multiprocessor system) or a system may include a cache memory.

Arrows such as 422 represent the system bus architecture of computer system 301. However, these arrows are illustrative of any interconnection scheme serving to link the subsystems. For example, speaker 420 could be connected to the other subsystems through a port or have an internal direct connection to central processor 402. The processor may include multiple processors or a multicore processor, which may permit parallel processing of information. The processor may include a graphical durable unit (GPU). The processor may include a GPU where parallel processing is desired. Computer system 301 shown in FIG. 4 is an example of a computer system suitable for use with the laparoscopic oximeter.

Computer software products may be written in any of various suitable programming languages, such as C, C++, C#, Pascal, Fortran, Perl, MATLAB (from Math Works, www.mathworks.com), SAS, SPSS, JavaScript, AJAX, Java, Python, Erlang, and Ruby on Rails. The computer software product may be an independent application with data input and data display modules. Alternatively, the computer software products may be classes that may be instantiated as distributed objects. The computer software products may also be component software, such as Java Beans (from Oracle Corporation) or Enterprise Java Beans (EJB from Oracle Corporation).

An operating system for the computer system may be one of the Microsoft Windows® family of systems (e.g., Windows 95, 98, Me, Windows NT, Windows 2000, Windows XP, Windows XP x64 Edition, Windows Vista, Windows 7, Windows 8, Windows 10, Windows 11, Windows CE, Windows Mobile, Windows RT), Symbian OS, Tizen, Linux, HP-UX, UNIX, Sun OS, Solaris, Mac OS 15, Apple IOS, Android, Alpha OS, or AIX. Other operating systems may be used. Microsoft Windows is a trademark of Microsoft Corporation.

Any trademarks or service marks used in this patent are the property of their respective owners. Any company, product, or service names in this patent are for identification purposes only. Use of these names, logos, and brands does not imply endorsement.

Furthermore, the computer system 301 may be connected to network 104 and may connect to other computer systems using this network. The network may be an intranet, internet, or the Internet, among others. The network may be a wired network (e.g., using copper wire), telephone network, packet network, an optical network (e.g., using optical fiber), a wireless network, or any combination of these. For example, data and other information may be passed between the laparoscopic oximeter and the computer system 301 using a wireless network using a protocol such as Wi-Fi (IEEE standards 802.11, 802.11a, 802.11b, 802.11e, 802.11g, 802.11i, 802.11n, 802.11ac, and 802.11ad, just to name a few examples), near field communication (NFC), radio-frequency identification (RFID), mobile or cellular wireless (e.g., 2G, 3G, 4G, 5G, 3GPP LTE, WiMAX, LTE, LTE Advanced, Flash-OFDM, HIPERMAN, iBurst, EDGE Evolution, UMTS, UMTS-TDD, 1×RDD, and EV-DO).

For example, signals from a computer may be transferred, at least in part, wirelessly to components or other computers.

In an implementation, with a Web browser executing on computer system 301, the web browser accesses a system on the World Wide Web (WWW) through a network such as the Internet. The Web browser is used to download Web pages or other content in various formats, including HTML, XML, text, PDF, and PostScript, and may be used to upload information to other parts of the system of the WWW. The Web browser may use uniform resource identifiers (URLs) to identify resources on the Web and hypertext transfer protocol (HTTP) in transferring files on the Web.

In other implementations, a human user or another computer system accesses the computer system 301 through either or both of native and nonnative applications. Native applications are locally installed on the particular computer system 301 and are specific to the operating system or one or more hardware devices of that computer system, or a combination of these. These applications (which are sometimes also referred to as “apps”) can be updated (e.g., periodically) via a direct internet upgrade patching mechanism or through an applications store (e.g., Apple iTunes and App store, Google Play store, Windows Phone store, and Blackberry App World store).

The computer system 301 can run in platform-independent, nonnative applications. For example, a client operating on the computer system can access other computer systems through a Web application. The computer system 301 can access the other computer systems from one or more servers using a network connection with the server or servers. Thereby, the computer system can load the Web application via a Web browser, which can be the client operating on the computer system. For example, a Web application can be downloaded from an application server over the Internet by a Web browser. Nonnative applications can also be obtained from other sources, such as a disk.

FIG. 5 shows an example of a client system 108 that can include a smartphone in which some of the various techniques described in this application may be implemented. The client system includes, for example without limitation, a display 503, a front-facing camera 513, a rear-facing camera (not shown), a speaker or an array of multiple speakers 511, an optional proximity sensor 509, and a multi-function virtual or physical button 510 (e.g., for accessing one or more menus or home screen, for obtaining fingerprints for authentication and/or authorization, and others).

The client system 108 can include a tablet computer, a mobile telephone, a desktop computer, a laptop computer, or another type of computing device. The client system can be a device of Teguar Corporation of Charlotte North Carolina, Apple Inc. of Cupertino California, Alphabet Inc. of Mountain View California, Microsoft Corporation of Redmond Washington, Sony Corporation of Tokyo Japan, Samsung Electronics Co. Seoul, South Korea, Ltd, Dell Technologies Inc. of Round Rock Texas, or other computer manufacturers.

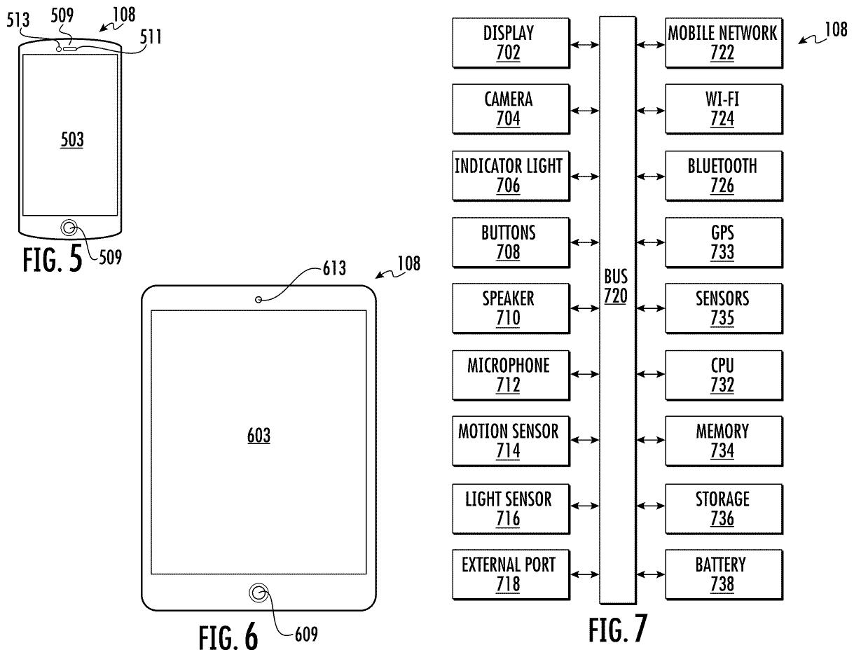

FIG. 6 shows an example client system 108 that can include a tablet computing device in which some of the various techniques described in this application may be implemented. The client system includes, for example without limitation, a display 603, a front-facing camera 613, a rear-facing camera (not shown), a speaker or an array of multiple speakers (not shown), a multi-function virtual or physical button 609 (e.g., for accessing one or more menus or home screen, for obtaining fingerprints for authentication or authorization, and others).

FIG. 7 shows a simplified diagram of a client system 108 in which some of the various techniques described in this application may be implemented. The client system of FIG. 7 may be the smartphone device shown in FIG. 5, the tablet computing device shown in FIG. 6, or another device.

Client system 108 includes a bus 720 that operates according to a bus protocol to transfer data between components inside the Client system or between computers (e.g., between the Client system and one or more other computing devices). The components inside Client system 108 and connected by bus architecture 720 may include a processor 732 (e.g., a central durable unit or CPU) that executes instructions from computer programs (including operating systems). The processor may include caches (e.g., L1, L2, or L3 cache, or any combination) and may utilize memory 734 (e.g., dynamic random-access memory or DRAM), as a cache where memory 734 may store instructions or computer code and data.

Client system 108 includes a storage device (e.g., persistent memory, a solid-state high-performance byte-addressable memory device, solid state drives, and others) 736 that stores computer programs and data such that it is typically persistent and provides more storage when compared to memory 734. Storage device 736 includes a removable storage device that provides mobility to computer programs and/or data that is stored in the storage device, in an implementation. The removable storage device can include a persistent memory or PRAM is a type of computer memory with the speed of RAM (random access memory), the retention of an SSD (solid state drive), and which remembers data even after powering off the device.

Memory 734 and storage device 736 provide examples of non-transitory computer readable storage media that may be utilized to store and retrieve computer programs incorporating computer codes that implement various implementations of the system, data for use with the system, and the like. Additionally or alternatively, a data signal embodied in a carrier wave (e.g., in a network including the Internet) may be another form of a computer-readable storage medium. These various components of a client system 108 may be powered by a battery 738 that may be charged via a charging circuit (not shown).

Client system 108 further includes a display 702, one or more cameras 704 (e.g., a front-facing camera, a rear-facing camera, and others), one or more indicator lights 706, one or more physical buttons, virtual buttons, or switches 708, and a speaker or an array of multiple speakers 710. Client system 108 further includes one or more microphones 712 (e.g., an array of a plurality of microphones for beamforming, actively cancelling noises, controlling directional audio outputs, and others), and one or more sensors 714 (e.g., proximity sensor, motion sensor, or other sensors). Client system 108 further includes one or more light sensors 716 (e.g., photosensors or photodiodes) 716 to sense and collect one or more characteristics of light. The light sensor may collect light intensity (e.g., representative of a human presence) and light color (e.g., red, green, or blue), illuminance in the unit of lux (e.g., the luminous flux as perceived by a surface and others), for example, to convert perceived light to electrical signals, in an implementation.

The client system 108 may include one or more display adapters (not shown) that function in conjunction with display 702 to, for example, to provide a user interface that accepts inputs and displays outputs. Client system 108 further includes one or more external ports 718. For example, a client system 108 may include a serial port that includes a serial communication interface through which information transfers in or out sequentially one bit at a time, and/or a parallel port that includes an interface allowing the client system 108 to transmit or receive data down multiple bundled cables to a peripheral device (e.g., a printer).

The external port 718 can operate according to one or more protocols for interfacing with external devices. For example, the external ports 718 can include a lightning port, a Thunderbolt port, a USB-C port, and other ports for interfacing with external devices. The client system 108 can further include one or more networking or telecommunication modules, such as a mobile network module 722 for communication with a cellular network, a Wi-Fi network module 724 for communication with a Wi-Fi network, a Bluetooth connection 726 for communication with other Bluetooth enabled devices, other network modules, or any combination of the modules.

The client system 108 can further include a GPS module 733 connected to bus 720 that collects GPS signals from GPS satellites. The GPS signals may be used by various applications that operate on the system. The client system can further include one or more sensors 735 that are connected to bus 720. The sensors can include various sensor types, such as tissue sensors of a user, heart rate, electrical conductivity of the user's skin, or other sensors.

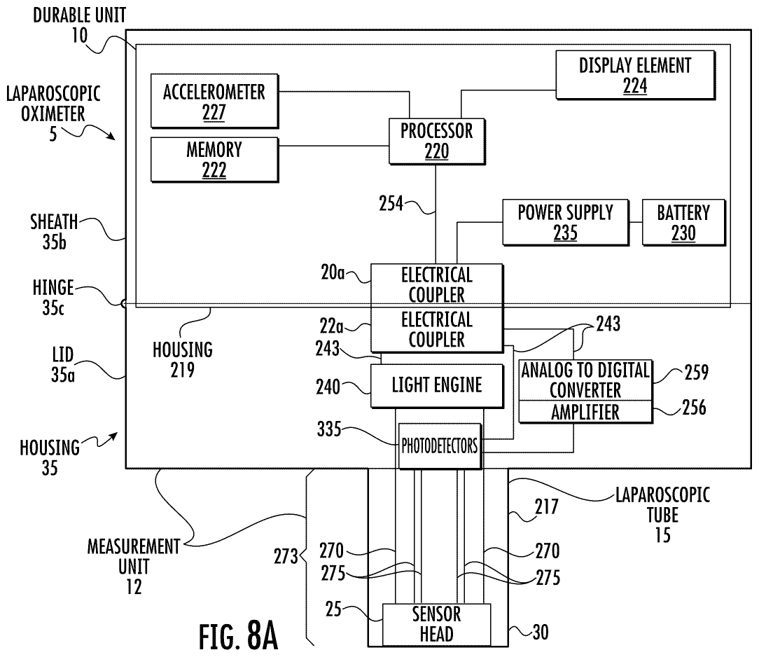

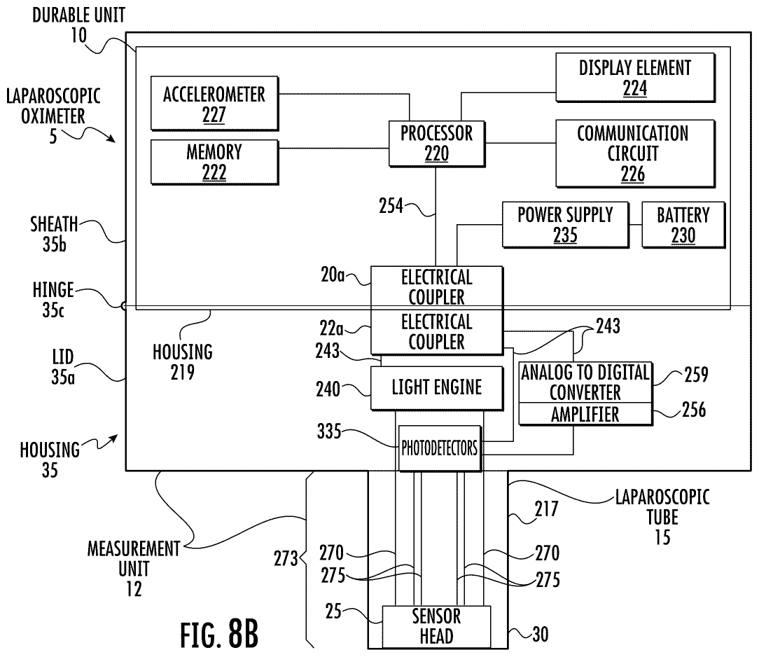

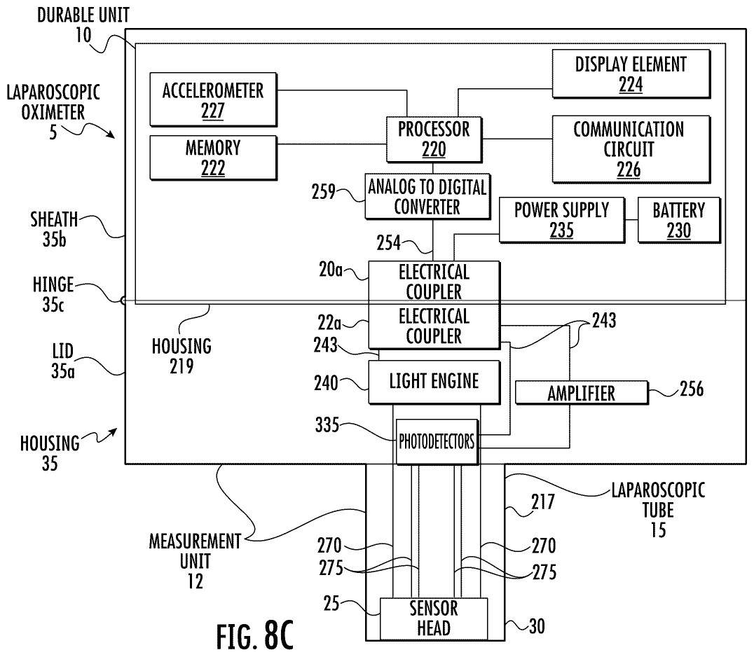

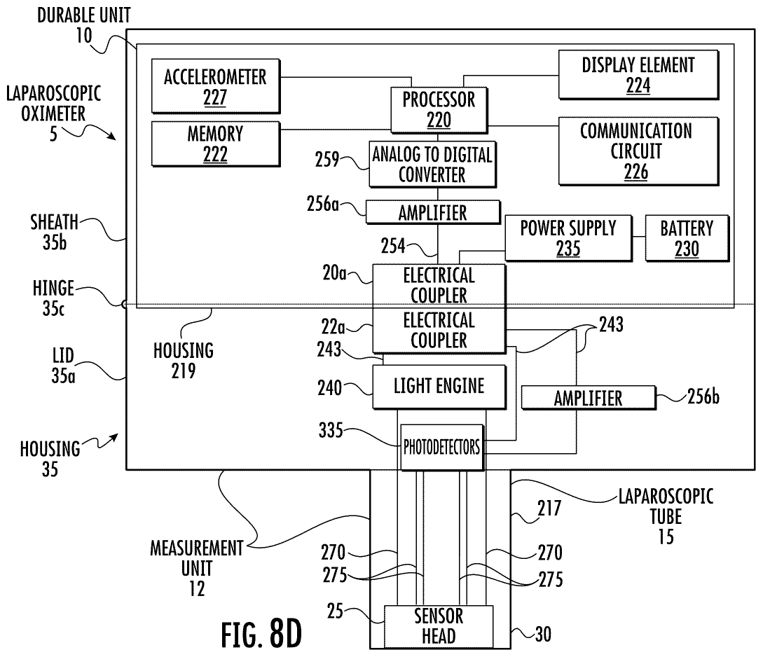

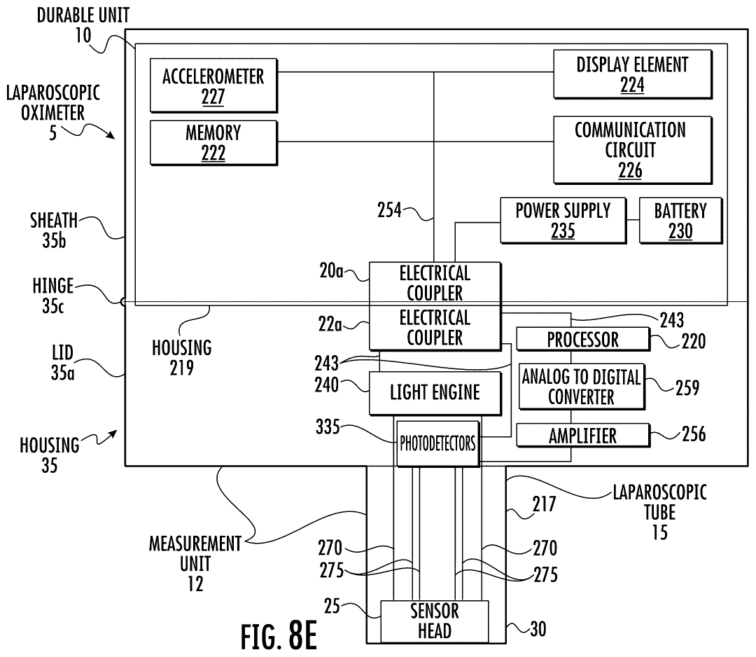

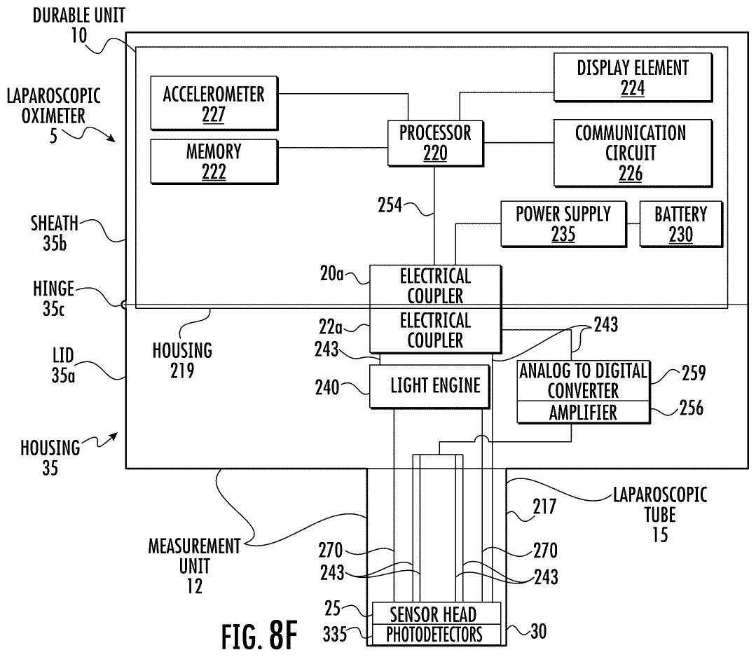

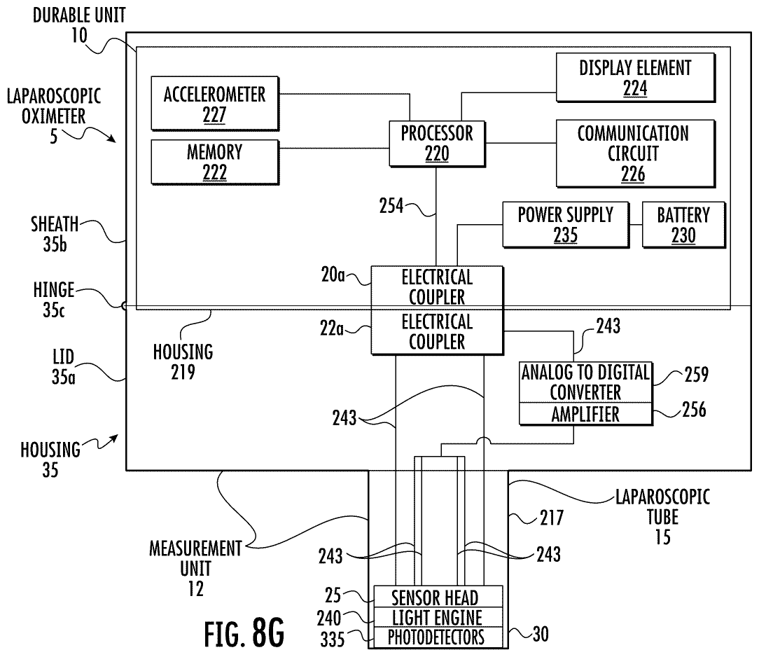

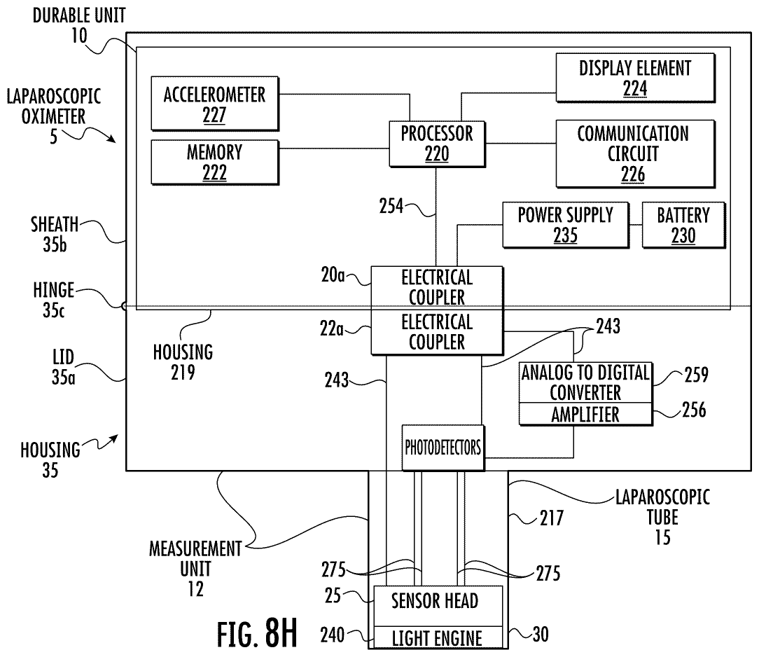

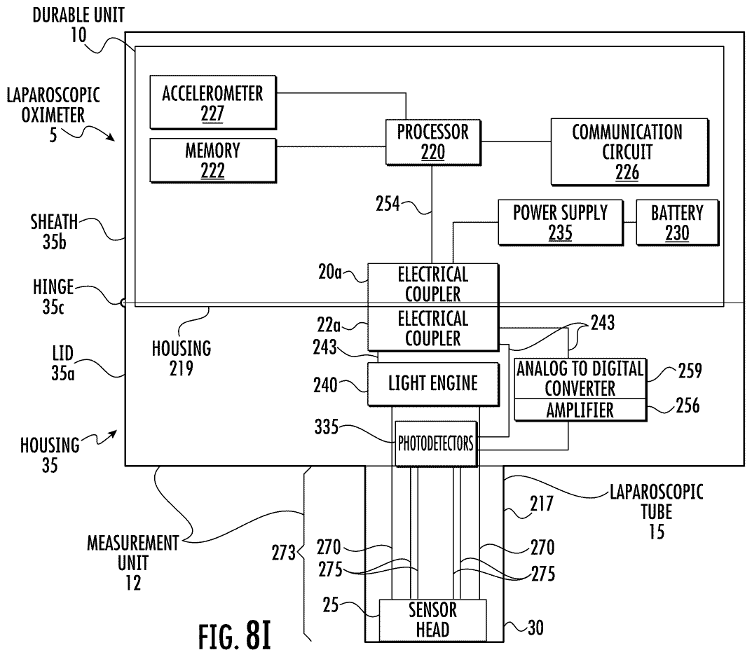

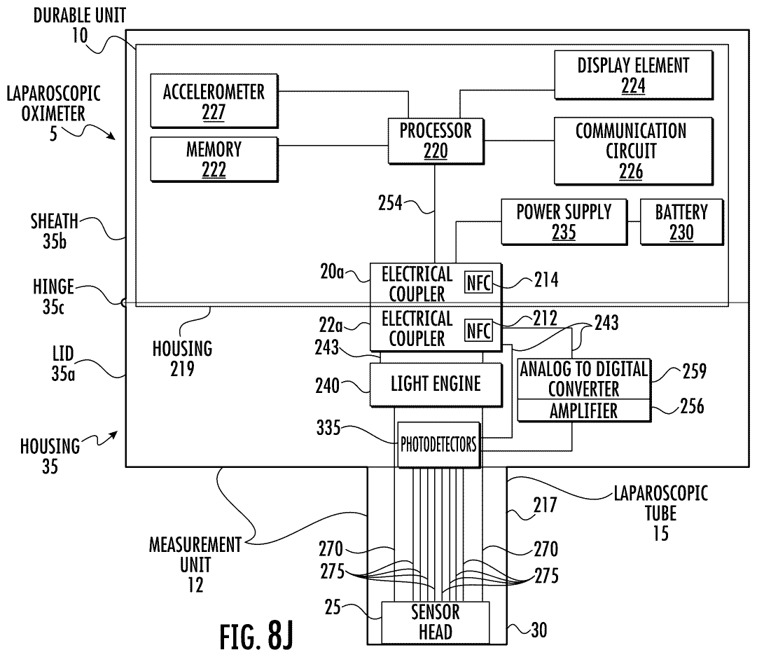

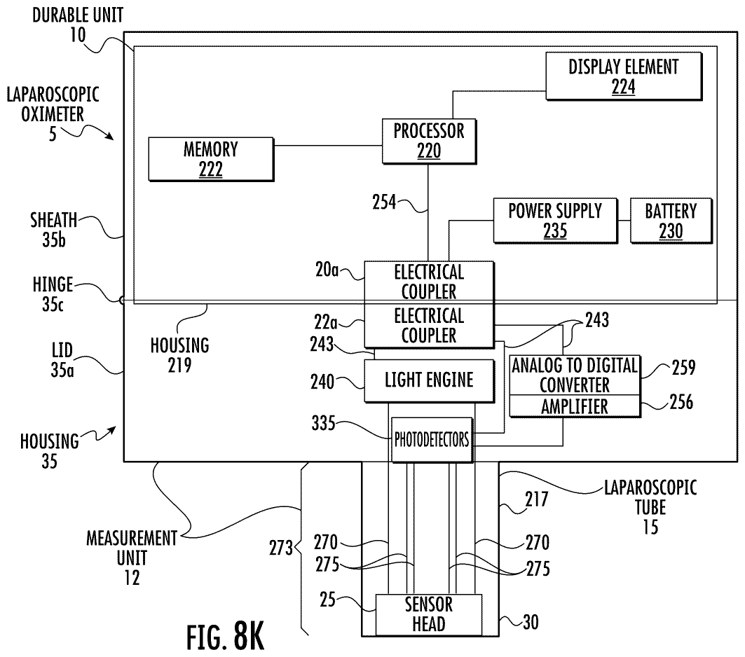

FIG. 8A is a diagram of laparoscopic oximeter 5, in an implementation. Laparoscopic oximeter 5 includes a durable unit 10 and a measurement unit 12. The measurement unit includes an elongated tube 15, such as a laparoscopic tube, and a housing 35. Durable unit 10 includes a housing 219 that houses a processor 220, a memory 222, a display 224, an accelerometer 227, a battery 230, a power supply circuit 235, one or more electrical conductors 254 (e.g., wires in a cable, such as a flex cable, electrical traces formed in a printed circuit board (PCB), or both), an electrical coupler 20a, other elements, or one or more of these elements in any combination. The electrical circuitry can be located on the same PCB as the electrical traces or a portion of the circuits can be located on another PCB. In an implementation where the durable unit includes display 224, the display element is visible from an exterior of the housing. In this implementation, the laparoscopic oximeter does not include a communication circuit.

The processor is connected to the accelerometer, memory, display, and electrical coupler via conductors 254. The circuits may be mounted on one or more PCBs included in the durable unit in housing 219, where the conductors are formed in or on the PCB.

The processor may be referred to as a processing circuit and can include one or more electrical components or circuits, such as a processor, microprocessor, microcontroller, a multi-core processor, application specific integrated circuit (ASIC), field programmable gate array (FPGA), multiplexers, standard cells, control logic (e.g., programmable logic, programmable logic device (PLD), CPLD, and others), memory, look-up tables, state machines, logic gates, digital signal processors (DSP), and others. In an implementation, the processing circuit performs operations in digital (e.g., Boolean logic). The circuitry can include one or more memories, such as a volatile memory (e.g., a RAM), a nonvolatile memory (e.g., a disk or FLASH), or other memory types.

The display can be a liquid crystal display (LCD) using a backlight, such as an LED to illuminate liquid crystals. The LCD display can be a twisted nematic type of LCD display, an in-plane switching LCD display, a vertical alignment type of LCD display, or other type of LCD display. The display can be an electrophoretic or electrochromic display.

The battery is connected to the power supply circuit, which is connected and supplies battery power to the processor, memory, display, accelerometer, and electrical coupler. The power supply circuit can be a DC-to-DC (direct current to direct current) converter circuit that converts the voltage output from the battery to a different voltage. The battery of the durable unit can include one or more of a variety of battery types, such as one or more disposable batteries (e.g., size AAA, AA, or other sizes) or one or more rechargeable batteries. Disposable batteries are discarded after their stored charge is depleted. Some disposable battery chemistry technologies include non-rechargeable lithium-ion, sodium-ion, alkaline, zinc-carbon, or silver oxide. The battery has sufficient stored charge to allow the use of the handheld device for several hours.

In implementations where the battery is rechargeable, the battery can be recharged multiple times after the stored charge is depleted. Some rechargeable battery chemistry technologies include nickel cadmium (NiCd), nickel metal hydride (NiMH), lithium ion (Li-ion), and zinc air. The battery can be recharged, for example, via a wireless charging system (e.g., a wireless charging system operating according to the Qi standard or another charging standard), via an AC adapter with a cord that connects to the handheld unit. The circuitry in the durable unit can include a recharger circuit (not shown) for recharging a rechargeable battery.

In an implementation, housing 35 includes a lid 35a, sheath 35b, and hinge 35c that hinge couples the lid and the sheath. The laparoscopic tube is coupled to an end portion of the lid. When the lid and sheath are open, the durable unit can be placed into the sheath and removed from the sheath. The lid and sheath, when closed, form a sealed interior space that the durable unit can be located in.

The durable unit 10 is configured for reuse, whereas the measurement unit 12 is configured to be disposed of after use with a single patient. The measurement unit 12 that includes the laparoscopic tube 15 and the housing 35 is sometimes referred to as a disposable unit because the measurement unit is intended to be disposed of after a single use, in an implementation. In an alternative implementation, the measurement unit 12 can be sanitized and reused after use with a patient.

The lid includes an interior space that houses an electrical coupler 22a, a light engine 240, an analog-to-digital converter (ADC) circuit 259, one or more amplifiers 256 (e.g., a transimpedance amplifier, a fixed gain amplifier, and a processor-controlled amplifier), photodetectors 259, other circuits, or any combination of these circuits. Processor 220 can couple to the light engine, ADC, amplifiers, photodetectors, or any combination of these circuits through electrical couplers 20a and 22a when the electrical couplers are electrically coupled.

More specifically, the electrical coupler 20a of the durable unit can electrically couple the durable unit to the measurement unit via electrical coupler 22a. The electrical couplers can be coupled and uncoupled when housing 35 is closed and open via the lid being closed onto the sheath and the lid being opened from the sheath. The electrical couplers can include mechanical elements that can mechanically couple or can aid the mechanical coupling of the durable unit to the measurement unit.

The laparoscopic tube includes a housing 217 having an interior space, source waveguides 270, detector waveguides 275, and a sensor head 25 located at a tip 30 of the laparoscopic tube. The housing can include an interior space that extends from a first end of the housing to a second end of the housing. Housing can be a tube having an elongated tube-shape and include the interior space, in an implementation. The interior space of the tube can have a fixed radius from the first end to the second end. The exterior surface of the tube can have a fixed radius from the first end to the second end. The housing can be formed of metal, such as stainless steel, titanium, or other metals, plastic-type materials, other types of materials, or any combination of these materials. Stainless steel allows for a relatively small outer diameter of the housing of the laparoscopic tube so that the housing can be placed inside a relatively small cannula or other type of port compared to a plastic housing having a larger diameter.

The housing 217 can have a variety of lengths 273 where the length of the housing extends from the location where the housing extends from the lid to the sensor head or to the face of the sensor head. The length of the housing can be from about be about 5 centimeters (e.g., about 2 inches) to about 40 centimeters (e.g., about 15.74 inches). In some implementations, the length of the housing is about 5.1 centimeters (e.g., about 2 inches), about 7.6 centimeters (e.g., about 3 inches), about 10.2 centimeters (e.g., about 4 inches), about 12.7 centimeters (e.g., about 5 inches, about 15.25 centimeters (e.g., about 6 inches), about 17.8 centimeters (e.g., about 8 inches), about 22.9 centimeters (e.g., about 9 inches), about 25.5 centimeters (e.g., about 10 inches), about 27.9 centimeters (e.g., about 11 inches), about 30.5 centimeters (e.g., about 12 inches), about 33 centimeters (e.g., about 13 inches), about 35.6 centimeters (about 14 inches), about 38.1 centimeters (e.g., about 15 inches), or other lengths.

Housing 217 of the laparoscopic tube, having a specific length, can be chosen for one or more specific medical procedures. For example, a housing of the laparoscopic tube having a length of about 5 centimeters to about 13 centimeters may be useful for an esophagectomy, where a part or the entire esophagus is removed. The laparoscopic oximeter can measure and display the oxygen saturation of the portions of tissue that will be connected following the removal of the portion or the entire esophagus to verify that the tissue is healthy for the connection of the tissue. The tissue for which oxygen saturation can be measured by the laparoscopic oximeter can include the stomach or a portion of the colon that will replace the removed esophagus.

The laparoscopic tube can be rigidly connected to the lid. The connection can be an adhered connection (e.g., an epoxy connection), a mechanical connection (e.g., a connection by friction, a connection by fasteners, or other connection types), or any combination of these connection types.

In an implementation, the sensor head is located in the tip portion 30 of a housing 217 of the laparoscopic tube 15 or can extend outward from the tip portion 30 of the housing. In an implementation, the sensor head is integrally formed with the housing. In an implementation, the sensor head is not integrally formed with the housing and can be a different material from the housing. The sensor head can be formed of a plastic type material, whereas the housing can be formed of metal, such as stainless steel or another metal. The sensor head can be a relatively rigid plastic type material, such as acrylic, polycarbonate, high-density polyethylene (HDPE), polyvinyl chloride (PVC), acrylonitrile butadiene styrene (ABS), polytetrafluoroethylene (PTFE), high-impact polystyrene (HIPS), acetal, resin, or other types of plastic material.

The face of the sensor head can be perpendicular to the longitudinal axis of the housing of the laparoscopic tube, in an implementation. In alternative implementations, the face of the sensor head is not perpendicular to the longitudinal axis of the housing of the laparoscopic tube. For example, the face can be angled at 85 degrees, 80 degrees, 75 degrees, 70 degrees, 65 degrees, 60 degrees, 55 degrees, 50 degrees, 45 degrees, or other angles from the longitudinal axis of the housing of the laparoscopic tube.

Laparoscopic oximeter 5 shown in FIG. 8A and described can include any combination of the circuits, circuit configurations, and waveguide configurations shown in FIGS. 8B-8J or other figures and described with respect to these figures. The operation of laparoscopic oximeter 5 is described further below.

Source waveguides 270 can include optical fibers or other types of waveguides. The source waveguides extend from the sensor head and through the interior space of housing 217 (e.g., an interior tubular space of the housing) into the interior space of the lid 35a that contains electrical coupler 22a, light engine 240, ADC 20d, amplifiers 256, and photodetectors 259. Detector waveguides 275 can include optical fibers or other types of waveguides. The detector waveguides extend from the sensor head and through the interior space of the housing into the interior space of the lid that contains electrical coupler 22a, light engine 240, ADC 20d, amplifiers 256, and photodetectors 259.

The sensor head includes a number of apertures formed in the sensor head. The apertures extend from a first end of the sensor head that is located in the interior space of the laparoscopic housing 217 of the laparoscopic tube 15 to a second end of the sensor head that is located outside of the interior space of the laparoscopic housing of the laparoscopic tube. The second end of the sensor head forms the face of the sensor head of laparoscopic oximeter 5. The apertures can have central axes that are parallel with a longitudinal axis of the laparoscopic tube where the longitudinal axis of the laparoscopic tube extends from a first end of the laparoscopic tube at the lid to a second end of the laparoscopic tube at the sensor head. The longitudinal axis can be parallel to the outer wall of housing 217 of the laparoscopic tube, such as when the diameter of the housing is constant.

Portions of the second ends of the source waveguides and second ends of the detector waveguides are located in the apertures. The second ends of the waveguides can be adhered inside the apertures, such as via epoxy.

More specifically, tip portions of the second ends of the source waveguides and tip portions of the second ends of the detector waveguides can be flush with the openings of the apertures of the sensor head at the face of the sensor head. The tip portions of the second ends of the source waveguides and detector waveguides can be located inside of the apertures behind the face of the sensor head or flush with the face of the sensor head. In an implementation where the ends of the source and detector waveguides are inside of the apertures formed in the sensor head, the open interior space of the apertures to which the waveguides do not extend can be filled with epoxy, where the epoxy covers the ends of the source and detector waveguides. The epoxy can be polished with the sensor head during manufacturing to form the face of the sensor head. In an implementation where the epoxy covers the ends of the source waveguides, the index of refraction of the epoxy can approximately match the index of refraction of the source waveguides to limit reflections between the epoxy and the waveguides. In an implementation where epoxy covers the ends of the detector waveguides, the index of refraction of the epoxy can approximately match the index of refraction of the detector waveguides to limit reflections between the epoxy and the waveguides. The epoxy located in the apertures of the sensor head can form waveguides that guide light from the source waveguides outward from the sensor head, such as into patient tissue or a tissue phantom, such as for calibration. The epoxy located in the apertures of the sensor head can also form waveguides that guide light that guide light that is collected by the sensor head (e.g., reflected from patient tissue, a tissue phantom material, or other material) to the detector waveguides.

Portions of the sensor head from which light exits the sensor head are sometimes referred to as source structures, and portions of the sensor head at which light enters the sensor head are referred to as detector structures. The source structure can include portions of the source waveguides (e.g., portions of the waveguides in the apertures formed in the sensor head), the epoxy that forms waveguides that receive light from the source waveguides, open space in the apertures, or other structures of the sensor head. The detector structures can include portions of the detector waveguides (e.g., portions of the waveguides in the apertures formed in the sensor head), the epoxy that forms waveguides that transmit light to the detector waveguides, open space in the apertures, or other structures of the sensor head.

First ends of the source waveguides are optically coupled, mechanically coupled, or both to the light source and second ends of the source waveguides are optically coupled, mechanically coupled, or both to the sensor head. The number of source waveguides is less than, more than, or equal to the number of detector waveguides. First ends of the detector waveguides are optically coupled, mechanically coupled, or both to the photodetectors and second ends of the detector waveguides are optically coupled, mechanically coupled, or both to the sensor head.

The number of optical fibers included in the source waveguides can be 1, 2, 3, 4, 5, 6, 7, 8, 9, 10, 11, 12, 13, 14 or more. The number of optical fibers included in the detector waveguides can be 1, 2, 3, 4, 5, 6, 7, 8, 9, 10 or more. In an implementation, the source waveguides include 2 waveguides (e.g., 2 optical fibers) and the detector waveguides include 4 waveguides (e.g., 4 optical fibers). In an implementation, the source waveguides include 2 waveguides (e.g., 2 optical fibers) and the detector waveguides include 8 waveguides (e.g., 8 optical fibers).

FIG. 8B is a diagram of laparoscopic oximeter 5, in an implementation. Laparoscopic oximeter 5 includes a communication circuit 226 that is coupled to durable unit 10. The communication circuit can be a transceiver circuit that is configured to wirelessly communicate via one or more wireless transmission protocols. The communication circuit can also be configured for hardwired communication via a communication port with another electronic device. Using one or more wireless transmission protocols, the laparoscopic oximeter can communicate with other devices, such as client system 108 via network 104, server system 106 via network 104, or computing device 301 via network 104 or via direct wireless communication. Laparoscopic oximeter 5 shown in FIG. 2B and described can include any combination of the circuits, circuit configurations, and waveguide configurations shown in FIGS. 8A-8J and described, other circuits, other circuit configurations, or other waveguide configurations. The operation of laparoscopic oximeter 5 is described further below.

In an implementation where communication circuit 226 provides for wireless communication, the communication circuit may operate according to one or more standard protocols such as Bluetooth or other local wireless network (IoT protocols such as Zigbee wireless mesh network, Z-wave, Bluetooth Low Energy (BLE or Bluetooth LE) aka Bluetooth 4.1. Communication circuit 226 may operate according to other IoT protocols, such as Advanced Message Queuing Protocol, AMQP, Cellular-2G, 3G, 4G/LTE, 5G, or others, Constrained Application Protocol or CoAP, Data Distribution Service or DDS, LoRa, LoRaWAN, Lightweight M2M (LWM2M) as a device management protocol designed for sensor networks and the demands of an M2M environment, Message Queuing Telemetry Transport or MQTT, Wi-Fi, XMPP Extensible Messaging and Presence Protocol for real-time human-to-human communication, Zigbee which has a longer range than BLE but a lower data rate than BLE, Z-Wave allowing smart devices to communicate with encryption and thereby providing a level of security to the IoT (Internet of Things) deployment, and others Bluetooth LE, colloquially BLE, formerly marketed as Bluetooth Smart) is a wireless personal area network technology.

Bluetooth mesh profiles use Bluetooth Low Energy to communicate with other Bluetooth Low Energy devices in the network. Each device may pass the information forward to other Bluetooth Low Energy devices creating a mesh effect.

In some implementations, communication circuit 226 operate via one or more of protocols such as 802.11x, 802.15 (e.g., 802.15.1 for WPAN/Bluetooth connections, 802.15.2 for coexistence connections, 802.15.3 for high-rate WPAN, 802.15.3b-2005, 802.15.3c-2009, 802.15.3d-2017, 802.15.3e-2017, 802.15.3f-2017, 802.15.4 for low-rate WPAN connections, 802.15.4a, 802.15.5, 802.15.6, 802.15.7 for visible light communication, 802.15.8 for Peer Aware Communications, 802.15.9 for Key Management Protocol, 802.15.10 for Layer 2 Routing, 802.15.13 for Multi-Gigabit/s Optical Wireless Communications, and others), DASH7 Alliance Protocol, Ultra-wideband (UWB), or ultraband.

802.15.1 may be used for WPAN or Bluetooth connections. More particularly, task group one may be based on Bluetooth technology. 802.15.1 defines physical layer (PHY) and Media Access Control (MAC) specification for wireless connectivity with fixed, portable and moving devices within or entering personal operating space. 802.15.2 is used where task group two addresses the coexistence of wireless personal area networks (WPAN) with other wireless devices operating in unlicensed frequency bands such as wireless local area networks (WLAN).

802.15.3 for high-rate WPAN comprises a MAC and PHY standard for high-rate (11 to 55 Mbit/s) WPANs. 802.15.3a was an attempt to provide a higher speed ultra-wideband PHY enhancement amendment to IEEE 802.15.3 for applications that involve imaging and multimedia. 802.15.3b-2005 amendment was released on May 5, 2006. It enhanced 802.15.3 to improve implementations and interoperability of the MAC. This amendment includes many optimizations, corrected errors, clarified ambiguities, and added editorial clarifications while preserving backward compatibility.

802.15.3c-2009 constitutes a millimeter-wave-based alternative physical layer (PHY) for the existing 802.15.3 Wireless Personal Area Network (WPAN) Standard 802.15.3-2003. 802.15.3d-2017: an alternative physical layer (PHY) at the lower THz frequency range between 252 GHz and 325 GHz for switched point-to-point links is defined in this amendment. Two PHY modes are defined that enable data rates of up to 100 gigabytes per second using eight different bandwidths between 2.16 GHz and 69.12 GHz. 802.15.3e-2017 constitutes an alternative physical layer (PHY), and a modified medium access control (MAC) layer is defined in this amendment. Two PHY modes have been defined that enable data rates up to 100 Gb/s using the 60 GHz band. MIMO and aggregation methods have been defined to increase the maximum achievable communication speeds. Stack acknowledgment has been defined to improve the medium access control (MAC) efficiency when used in a point-to-point (P2P) topology between two devices.

802.15.3f-2017 extends the RF channelization of the millimeter wave PHYs to allow for use of the spectrum up to 71 GHz. 802.15.3f was initiated because several regulatory domains extended the licensed exempt 60 GHz bands up to 71 GHz. 802.15.4 for Low Rate WPAN connections deals with low data rate but very long battery life (months or even years) and very low complexity. The standard defines both the physical (Layer 1) and data-link (Layer 2) layers of the OSI model. IEEE 802.15.4a (formally called IEEE 802.15.4a-2007) is an amendment to IEEE 802.15.4 specifying additional physical layers (PHYs) to the original standard. The principal interest was in providing higher precision ranging and localization capability (1 meter accuracy and better), higher aggregate throughput, adding scalability to data rates, longer range, and lower power consumption and cost.

802.15.5 provides the architectural framework enabling WPAN devices to promote interoperable, stable, and scalable wireless mesh networking. This standard is composed of two parts: low-rate WPAN mesh and high-rate WPAN mesh networks. The low-rate mesh is built on IEEE 802.15.4-2006 MAC, while the high-rate mesh utilizes IEEE 802.15.3/3b MAC. The common features of both meshes include network initialization, addressing, and multi-hop unicasting. In addition, the low-rate mesh supports multicasting, reliable broadcasting, portability support, trace route and energy saving function, and the high-rate mesh supports multi-hop time-guaranteed service. The IEEE 802.15.6 task group approved a draft of a standard for Body Area Network (BAN) technologies. The draft was approved on 22 Jul. 2011 by Letter Ballot to start the Sponsor Ballot process. Task Group 6 was formed in November 2007 to focus on a low-power and short-range wireless standard to be optimized for devices and operation on, in, or around the human body (but not limited to humans) to serve a variety of applications, including medical, consumer electronics, and personal entertainment.

802.15.7 may be used for visible light communication with several new PHY layers and MAC routines to support optical camera communications (OCC) and light fidelity (Li-Fi). In March 2017, the 802.15 Working Group decided to continue 802.15.7 with OCC only, which is broadcast only, and to create a new task group 802.15.13 to work on a new standard for Li-Fi, which needed a revised MAC layer, besides new PHYs.

DASH7 Alliance Protocol (D7A) is an open-source wireless sensor and actuator network protocol, which operates in the 433 MHz, 868 MHz, and 915 MHz unlicensed ISM band/SRD band. DASH7 provides multi-year battery life, a range of up to 2 km, low latency for connecting with moving things, a very small open-source protocol stack, AES 128-bit shared-key encryption support, and data transfer of up to 167 kbit/s. The DASH7 Alliance Protocol is the name of the technology promoted by the non-profit consortium called the DASH7 Alliance. Ultra-wideband (UWB, ultra-wideband, ultra-wide band, and ultraband) is a radio technology that can use a very low energy level for short-range, high-bandwidth communications over a large portion of the radio spectrum. UWB was proposed for use in personal area networks, and appeared in the IEEE 802.15.3a draft PAN standard.

FIG. 8C shows an implementation of laparoscopic oximeter 5 where ADC 259 is located in durable unit 10. The ADC is electrically connected between amplifier 256 and processor 220 via the electrical couplers 20a and 22a when the couplers are connected. Laparoscopic oximeter 5 shown in FIG. 8C and described can include any combination of the circuits, circuit configurations, and waveguide configurations shown in FIGS. 8A-8J and described, other circuits, other circuit configurations, or other waveguide configurations. The operation of laparoscopic oximeter 5 is described further below.

FIG. 8D shows an implementation of laparoscopic oximeter 5 where ADC 259 is located in durable unit 10 and at least one amplifier 256a is located in the durable unit. Amplifier 256a can include a fixed-gain amplifier, a processor-controlled amplifier, or both of these types of amplifiers. At least one amplifier 256b is located in the measurement unit 12, such as in the lid 35a of housing 35. Amplifier 256b can include a fixed gain amplifier, a processor-controlled amplifier, a transimpedance amplifier (TIA), or a combination of these amplifiers, such as a fixed gain amplifier and a TIA, or a processor-controlled amplifier and a TIA.

The ADC can be electrically connected between amplifiers 256a-256b and processor 220 via the electrical couplers 20a and 22a when the couplers are connected. In an implementation, the ADC can be electrically connected between amplifiers 256a-256b when the couplers are connected. The ADC can be located in the durable unit 10 or in the measurement unit 12, such as in the lid 35a of the housing 35. In another implementation, one or more of the amplifiers are positioned in the durable unit 10 and are coupled between the ADC and the photodetectors. Laparoscopic oximeter 5 shown in FIG. 8D and described can include any combination of the circuits, circuit configurations, and waveguide configurations shown in FIGS. 8A-8J and described, other circuits, other circuit configurations, or other waveguide configurations. The operation of laparoscopic oximeter 5 is described further below.

FIG. 8E shows an implementation of laparoscopic oximeter 5 where the processor is located in the housing 35, such as in lid 35a. The processor is coupled to electrical coupler 22a, light engine 140, ADC 259, amplifier 257, photodetectors 335, other circuits that may be in housing 35 (e.g., lid 35a), or any combination of these circuits by electrical conductors 256. The processor may be coupled to circuits housed in housing 219 through the electrical couplers 20a and 22a when the electrical couplers are connected. In an implementation, memory 222 is housed in the housing 35 (e.g., in lid 35a) and is coupled to the processor in housing 35. In an implementation, accelerometer 227 is housed in housing 35 (e.g., in lid 35a) and is coupled to the processor in housing 35. In an implementation, communication circuit 226 is housed in housing 35 (e.g., in lid 35a) and is coupled to the processor in housing 35. In an implementation, the power supply 235 and battery 230 are housed in the housing 35 (e.g., in lid 35a) and are coupled to the processor and the other circuits in housing 35. Laparoscopic oximeter 5 shown in FIG. 8E and described can include any combination of the circuits, circuit configurations, and waveguide configurations shown in FIGS. 8A-8J and described, other circuits, other circuit configurations, or other waveguide configurations. The operation of laparoscopic oximeter 5 is described further below.

FIG. 8F shows an implementation of laparoscopic oximeter 5 where the photodetectors 335 are located in the sensor head and the light engine is located in the lid 35a of housing 35. Electrical conductors 243 connect the photodetectors to the processor. The electrical conductors can extend from the processor 220 to electrical conductors 220 and through electrical conductor 20a to electrical conductor 22a. From electrical conductors 22a, the electrical conductors extend through the interior space of the laparoscopic tube to the photodetectors. In an implementation where the processor is in the measurement unit, such as in the lid, the electrical conductors can extend from the processor to the photodetectors through the interior space of the laparoscopic tube. Laparoscopic oximeter 5 shown in FIG. 8F and described can include any combination of the circuits, circuit configurations, and waveguide configurations shown in FIGS. 8A-8J and described, other circuits, other circuit configurations, or other waveguide configurations. The operation of laparoscopic oximeter 5 is described further below.

FIG. 8G shows an implementation of laparoscopic oximeter 5 where the light engine 240 and the photodetectors 335 are located in the sensor head. Electrical conductors 243 connect the light engine and the photodetectors to the processor. The electrical conductors can extend from the processor 220 to electrical conductors 220 and through electrical conductor 20a to electrical conductor 22a. From electrical conductors 22a, the electrical conductors extend through the interior space of the laparoscopic tube to the light engine. In an implementation where the processor is in the measurement unit, such as in the lid, the electrical conductors can extend from the processor to the light engine through the interior space of the laparoscopic tube. Electrical conductors 277 connect the photodetectors to the processor as described above. Laparoscopic oximeter 5 shown in FIG. 8G and described can include any combination of the circuits, circuit configurations, and waveguide configurations shown in FIGS. 8A-8J and described, other circuits, other circuit configurations, or other waveguide configurations. The operation of laparoscopic oximeter 5 is described further below.

FIG. 8H shows an implementation of laparoscopic oximeter 5 where the light engine 240 is located in the sensor head and the photodetectors are located in the lid 35a of housing 35. Laparoscopic oximeter 5 shown in FIG. 8H and described can include any combination of the circuits, circuit configurations, and waveguide configurations shown in FIGS. 8A-8J and described, other circuits, other circuit configurations, or other waveguide configurations.

FIG. 8I shows an implementation of laparoscopic oximeter 5 where the oximeter does not include a display. In this implementation, the processor can control the communication circuit to transmit oximeter information to a client system 108, server system 106, or computer system 310 for processing and display; for processing, display, and storage; for processing and transmission to one or more other devices for display; or for processing and transmission to one or more other devices for display and storage. Laparoscopic oximeter 5 shown in FIG. 8I and described can include any combination of the circuits, circuit configurations, and waveguide configurations shown in FIGS. 8A-8J and described, other circuits, other circuit configurations, or other waveguide configurations. The operation of the various implementations laparoscopic oximeter 5 is described further below.

FIG. 8J shows an implementation of laparoscopic oximeter 5 that includes a first radio frequency (RF) communication device 212 (e.g., near field communication (NFC) device) located in the measurement unit 12 (e.g., in housing 35) and a second RF communication device 214 (e.g., second NFC device) located in the durable unit 12 (e.g., housing 219). NFC device 212 can be located in the lid 35a of housing 35, such as near coupled electrical coupler 22a. NFC device 212 can be located on a PCB (not shown) that is located in the lid 35a, such as on a PCB on which the electrical coupler is located. Other circuits located in the lid can be located on the PCB. NFC device 214 can be located at an end of the housing 219, such as near electrical coupler 20a. The NFC device 214 can be located on a PCB (not shown) that is located near the end of housing 219, such as on a PCB on which electrical coupler 20a is located. Other circuits located in housing 219 can be located on the PCB.

When the durable unit is located in housing 35 and the lid 35a and sheath 35b are closed, NFC devices 212 and 214 are relatively close to each other, such that these devices can communicate through RF transmissions. NFC device 212 can include a memory that stores information that can be read by NFC device 214. In an implementation, NFC device 212 is configured to store calibration information for the laparoscopic tube that can be determined during the manufacturing of the tube. NFC device 214 can include a tag writer circuit that can write information to NFC device 212. Laparoscopic oximeter 5 shown in FIG. 8J and described can include any combination of the circuits, circuit configurations, and waveguide configurations shown in FIGS. 8A-8J and described, other circuits, other circuit configurations, or other waveguide configurations. The operation of the various implementations laparoscopic oximeter 5 is described further below.

FIG. 8K shows an implementation of laparoscopic oximeter 5 where the oximeter does not include an accelerometer. Laparoscopic oximeter 5 shown in FIG. 8K and described can include any combination of the circuits, circuit configurations, and waveguide configurations shown in FIGS. 8A-8J and described, other circuits, other circuit configurations, or other waveguide configurations. The operation of the various implementations laparoscopic oximeter 5 is described further below.

In an implementation, the processor controls the generation of light by the light engine, which transmits the generated light into the source waveguides 270 for further transmission from the sensor head. The light can be transmitted from the sensor head into patient tissue so that the laparoscopic oximeter 5 can make oximetry measurements, into a tissue phantom for calibration of the durable unit, calibration of the measurement unit (the light engine and the photodetectors), or both.

The detector waveguides can detect the reflected light after reflection from or transmission through patient tissue or a tissue phantom and transmit the received light to the photodetectors. The photodetectors convert the received light into detector responses (e.g., analog electrical signals). The photodetectors are electrically coupled to the amplifiers, which can receive the detector response generated by the photodetectors. Thereafter, the amplifiers amplify the detector responses.

For example, one or more TIAs amplify the detector responses. In an implementation, multiple photodetectors are connected to a single TIA via a multiplexer. In an implementation, the photodetectors are connected to the TIAs in a one-to-one manner. The TIAs are adapted to receive the detector responses generated by the photodetectors and convert current signals of the detector responses to amplified voltage signals. A fixed gain amplifier and a processor-controlled amplifier may be connected to the TIA and amplify the TIA voltage signals to levels that the ADCs can receive and convert to digital signals that are usable by the processor.

In the implementation shown in FIGS. 8A-8J, for example, the TIAs may amplify low current signals generated by one or more photodetectors 335 to stable voltage signals, the fixed gain amplifier may amplify the stable voltages output from the TIAs to higher voltages, and the processor-controlled amplifier may amplify the higher voltages output by the fixed gain amplifier to voltages usable by the ADC and processor.

The ADCs sample the analog signals at a sampling rate. For example, the sampling rate can be on the order of kilohertz, such as about 200-300 kilohertz. The measurement rate that the processor operates on the digitized signals is less than the sampling rate. The measurement rate that the processor operates on the digitized signals is about 1-3 hertz (depending on the conditions of the laparoscopic oximeter). In other implementations, the measurement rate can be above 3 hertz, such as from about 4 hertz to about 1 kilohertz. Generally, the faster the sampling rate of the ADCs and the operating frequency of the processor, the more power that is consumed, which is a consideration for a battery-operated device, and also the data generated increases with the sample rate.

When using a measurement rate of about 0.33 hertz to about 3 hertz, the amount of data can be transmitted wirelessly by the communication circuit to other devices (e.g., a computer or a display) using technologies such as Bluetooth and Wi-Fi (and others mentioned in this patent) without data loss. In other implementations, a proprietary wireless technology can be used, such as when higher sampling rates, measurement rates, or both are desired.

The processor can apply one or more calibration calculations to digitized detector responses received from the ADCs, such as performing one or more calibration steps on the data. Calibration can include calibrating the digitized detector responses for inherent discrepancies in the intensity of light emitted by the LEDs of the light engine, for inherent discrepancies in the detection sensitivity of the photodetectors, or both, or other correlation calculations.

Calibration information for the measurement unit, such as the LEDs, driver circuit for the LEDs, photodetectors, a combination of these circuits, or other circuits in the measurement unit, can be predetermined during manufacturing of the measurement unit. Calibration information can be stored in a memory of the measurement unit for use when the laparoscopic oximeter makes oximetry measurements.

In an implementation, the processor is adapted to use spatially resolved spectroscopy techniques for determining oximetry information, such as blood oxygen saturation, of tissue from the detector signals generated by the photodetectors. Spatially resolved spectroscopy does not use dyes introduced into patient tissue for making oximetry measurements. Spatially resolved spectroscopy is facilitated by the distances between the source structure and the detector structure of the sensor head. For example, at least one source structure to detector structure spacing at the face of the sensor head is less than about 1.5 millimeters and another source structure to detector structure spacing is greater than about 2.5 millimeters. The oximetry information generated by spatially resolved spectroscopy techniques, using near infrared spectroscopy (NIRS) laparoscopically or using NIRS laparoscopically, including visible wavelengths, can include perfusion information for tissue that is being measured by the laparoscopic oximeter, such as an interior organ of a body.

Spatially resolved spectroscopy is further facilitated by the memory storing and the processor using a number of simulated reflectance curves, where each of the reflectance curves represents an absorption coefficient and a scattering coefficient (e.g., a reduced scattering coefficient) for the particular configuration of source structures and detector structures of the sensor head for simulated tissue.

The simulated reflectance curves include reflectance intensities (e.g., in arbitrary units) for light reflected from simulated tissue for a variety of wavelengths emitted from the laparoscopic oximeter and a variety of tissue conditions, such as a variety of oxygen saturations. The simulated reflectance curves can be of simulated tissue using a Monte Carlo simulation method, a diffusion approximation method, or other simulations. The simulated reflectance curves are determined using a computing device that is separate from the laparoscopic oximeter. The computer device uses simulated tissue to simulate the simulated reflectance curves. Further, the simulation performed by the computer device uses the spacings between the sources and detector, the spacings between the sources, the spacings between the detectors, or any combination of these spacings for the laparoscopic oximeter when generating the simulated reflectance curves. Thus, the simulated reflectance curves are generated for the laparoscopic oximeter having the described source and detector configuration. The simulated reflectance curves can be stored in a memory of the durable unit during the manufacturing of the durable unit for use by the processor to determine oximetry values from oximetry measurements. The simulated reflectance curves can be stored in a memory of the lid during the manufacturing of the measurement unit when the lid is configured to determine oximetry values from oximetry measurements. The simulated reflectance curves can be stored in a memory of a client system 108, for example, when the client system is configured to determine oximetry values from oximetry measurements.

The processor can determine one or more of the simulated reflectance curves that best fit (e.g., lowest fit error determined by a fit method, such as a least squares fit method or other fit methods) the reflectance data generated by the detectors. The processor can then determine one or more absorption coefficients and one or more scattering coefficients (e.g., reduced scattering coefficient) for the tissue from the one or more simulated reflectance curves that best fit the reflectance data. From the absorption coefficient, the processor can then determine other oximetry information for the measured tissue, such as oxygen saturation. The source structure to detector structure distances of the sensor head facilitate that the absorption coefficient and the scattering coefficient (e.g., reduced scattering coefficient) can be determined from the simulated reflectance curves, where these coefficients are mathematically independent. Because the absorption coefficient and the scattering coefficient (e.g., the reduced scattering coefficient) are mathematically independent, further tissue measurements, further mathematical determinations, or both can be avoided via the use of such spatially resolved spectroscopy. Mathematical independence of the absorption coefficient from the scattering coefficient allows for a determination by the processor an absolute oxygen saturation value versus a relative oxygen saturation value. Relative oxygen saturation values can be determined, however, by the processor using two or more absolute oxygen saturation values.

In an implementation where the laparoscopic oximeter is used for making oximetry measurements of tissue with melanin, multiple sets of simulated reflectance curves can be stored in the oximeter or in a computer system 301, such as a client system 108. Each set of stored simulated reflectance curves can include information for different absorption coefficient for tissue having different melanin concentrations. For example, if the oximeter is used for making an oximetry measurement of skin, tissue in the inner ear, or retina, the oximeter can store multiple sets of simulated reflectance curves for different melanin concentrations. can be stored. Melanin concentration of tissue can be determined by various method, such as a spectrometer or matching color cards to tissue. Information determined about melanin concentration can be used by the oximeter to select a particular set of simulated reflectance curves for the particular tissue and used by the processor to determine oxygen saturation information for measured tissue or other information for the tissue.

Calculated oximetry measurement values can thereafter be displayed on the display of the laparoscopic oximeter. For example, oxygen saturation values can be displayed on the display of the laparoscopic oximeter as numbers, in a graph, or both of these display formats.

The following U.S. patent applications are incorporated by reference along with all other references cited in this application: Ser. Nos. 13/887,130, 13/887,220, 13/887,213, 13/887,178, and 13/887,152, filed May 3, 2013; Ser. No. 13/965,156, filed Aug. 26, 2013; Ser. No. 15/493,132, 15/493,111, 15/493,121, filed Apr. 20, 2017; Ser. No. 15/494,444, filed Apr. 21, 2017; Ser. No. 15/495,194, 15/495,205, and 15/495,212, filed Apr. 24, 2017; Ser. No. 15/652,201, filed Jul. 17, 2017; Ser. No. 17/146,194, 17/146,197, and 17/146,201, file Jan. 11, 2021; U.S. Pat. No. 17,146,194, filed Jan. 11, 2021; Ser. No. 18/047,627, filed Oct. 18, 2022; Ser. No. 19/012,826, filed Jan. 7, 2025; U.S. patent application Ser. Nos. 19/012,790, 19/012,792, 19/012,826, and 19/012,833, filed Jan. 7, 2025; and 19/014,124, filed Jul. 31, 2025.

The above applications describe various laparoscopic oximeters and oximetry operations, such as spatially resolved spectroscopy, and the discussion in the above applications can be combined with aspects of the described implementations in this application, in any combination.

In an implementation, the processor in the laparoscopic element controls the transmission of oximetry measurement information to computer system 301. Computer system 301 can be a local computer system that is in the same room (e.g., operating room) where the laparoscopic element is used and is connected to the computer system via a wireless communication link, such as a Bluetooth link. In this implementation, computer system 301 is a smartphone, a tablet computer, a laptop computer, a desktop computer, a console, or another computing system. Computer system 301 can be a client system 108, where the client system 108 is connected to the computer system via the Internet 104 or another network. Computer system 301 can be a server system 106, where the server system 106 is connected to the computer system via the Internet or another network.

The oximetry measurement information can be digitized oximetry information that is digitized the analog-to-digital converter and packetized by the processor. The processor can include a microprocessor, a microcontroller, a GPU, a control logic circuit (e.g., an FPGA, a PLD, or other logic circuit), an ASIC, another circuit, or any combination of these circuits.

The oximetry measurement information can include unprocessed oximetry measurement information, partially processed oximetry measurement information, or processed oximetry measurement information. Unprocessed oximetry measurement information can include information that is digitized and packetized but otherwise unprocessed. For example, the unprocessed oximetry measurement information may not be calibrated, an oxygen saturation value may not be determined from the information, and no other mathematical processing may be performed on the information.

Partially processed oximetry measurement information can include information that is digitized and packetized where processing has occurred but an oxygen saturation value has not been determined from the information, where some intermediate values in the calculation process for determining an oxygen saturation value have been performed, such as determining a reflection coefficient, a scattering coefficient, a reduced scattering coefficient, where the information has undergone a calibration process, wherein the information has been amplified, or other partial processing has occurred.

Processed oximetry measurement information can include information where a percentage of saturated hemoglobin is calculated, where a percentage of unsaturated hemoglobin is calculated, where an oxygen saturation value is calculated, where calibration information is calculated for the laparoscopic oximeter, measurement unit, or sensor head, where a reflection coefficient, a scattering coefficient, or a reduced scattering coefficient are determined, or where another portion of oximetry information is calculated.

When unprocessed or partially processed oximetry information is transmitted from the laparoscopic oximeter to a computer system 301, the computer system can process the oximetry information to determine one or more of the processed oximetry information described immediately above from the received oximetry measurement information. Thereafter, one or more pieces of the processed oximetry information (e.g., an oxygen saturation value) can be displayed on the display of the computer system. In an implementation, the processed oximetry information can be displayed as a graph where the processed oximetry information is displayed for a period of time. For example, oxygen saturation values can be displayed against time on a graph on the display of the computer system.

In an implementation, the processed oximetry information (e.g., one or more oxygen saturation values) calculated by computer system 301 is transmitted to the laparoscopic oximeter for display on the display of the oximeter. The processed oximetry information can be displayed as numbers, in a graph, in text, as an average, or a combination of these presentation types.

The computer system 301 can transmit the processed oximetry information to the laparoscopic oximeter via network 104 (e.g., the Internet or other network and a Wi-Fi router or ethernet connection), via an RF communication (e.g., a Bluetooth communication), or a combination of these types of communications.

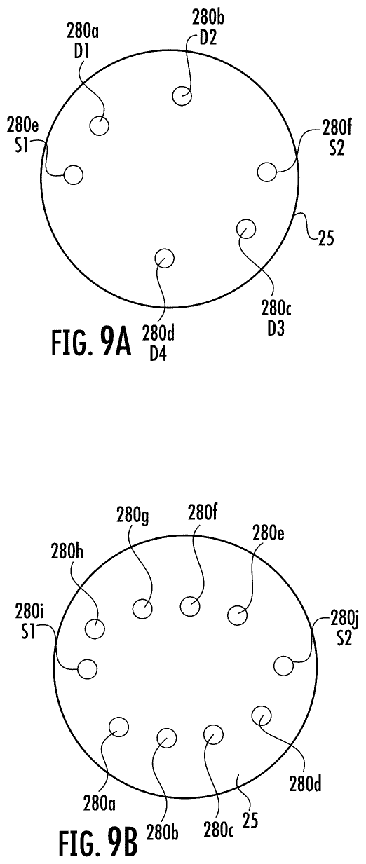

FIG. 9A shows an end view of the sensor head 25 of the laparoscopic oximeter, in an implementation. The sensor head includes two source structures 280e (S1)-280f (S2) and four detector structures 280a (D1), 280b (D2), 280c (D3), and 280d (D4), in an implementation. The sensor head can include more or fewer source structures and more or fewer detector structures. For example, the sensor head can include one source structure and two detector structures, two source structures and one detector structure, or other numbers of source structures and detector structures. The source structures emit light (visible light, infrared light, or a combination of these wavelengths) into waveguides 270 of the laparoscopic tube. The light is thereafter transmitted from the waveguides at the sensor head into tissue (e.g., patient tissue or a tissue proxy). The detector structures detect light reflected from tissue in response to the transmitted light.

The sources and detector structures can be arranged in a circle or in an arrangement that is not a circle. The detector structures can be arranged in a circle where the source structures are not on the circle. The detector structures can be arranged in an arrangement that is not a circle. Pairs of detector structures can be symmetrically arranged about a point on a line where the source structures are on the line. The point can be a midpoint on the line between the source structures. In an implementation, detector structures 280a and 280c can be arranged symmetrically about the point on the line, and detector structures 280b and 280d are arranged symmetrically about the point on the line. In an implementation, detector structures 280a and 280c can be arranged symmetrically about the point on the line, where detector structures 280b and 280d are not arranged symmetrically about the point on the line. In an implementation, detector structures 280b and 280d can be arranged symmetrically about the point on the line, where detector structures 280a and 280c are not arranged symmetrically about the point on the line.

Table 1 below includes information for the distances between the source structures and the distance between the source structures and detector structures in millimeters, in an implementation. In alternative implementations, the source structure to source structure distance can be different, the source structure to detectors structure distances can be different, or both can be different from the distances shown in table 1.

TABLE 1

| Source Structure Pair and | |
|---|---|
| Source Structure - Detector Structure Pairs | Distance Millimeters |
| S1-S2 | 4.3310 |
| S1-D1 | 1.4463 |
| S1-D2 | 3.1812 |
| S1-D3 | 4.0439 |
| S1-D4 | 2.3166 |
| S2-D1 | 4.0439 |
| S2-D2 | 2.3166 |
| S2-D3 | 1.4463 |
| D2-D4 | 3.1812 |

FIG. 9B shows an end view of the sensor head 25, in an implementation. The sensor head includes source structures 280i-280j and includes eight detector structures 280a-280h, in an implementation. The sensor head can include more or fewer source structures and more or fewer detector structures. The source structures emit light from waveguides 270 of the laparoscopic tube and the detector structures detect light reflected from tissue, such as patient tissue or a tissue proxy.

The sources and detector structures can be arranged in a circle or in an arrangement that is not a circle. The detector structures can be arranged in a circle where the source structures are not on the circle. The detector structures can be arranged in an arrangement that is not a circle. Pairs of detector structures can be symmetrically arranged about a point on a line where the source structures are on the line. The point can be a midpoint on the line between the source structures. In an implementation, detector structures 280a and 280e can be arranged symmetrically about the point on the line, detector structures 280b and 280f are arranged symmetrically about the point on the line, detector structures 280c and 280g are arranged symmetrically about the point on the line, and detector structures 280d and 280h are arranged symmetrically about the point on the line

The source structures and detector structures can include portions of the waveguides (e.g., portions of the waveguides located in the apertures formed in the sensor head, the tips of the terminal ends of waveguides, or both), other waveguides (e.g., epoxy located in the apertures) that optically couple to the waveguides, the apertures, the sidewalls of the apertures, the openings of the aperture on the face of the sensor head, other structures, or any combination of these structures.

This detailed description describes examples of implementations with specific measurements, angles, values, dimensions, shapes, and orientations. These example implementations are not intended to be exhaustive or to limit the described implementations to the precise form described.

The measurements, for example, in millimeters or centimeters are approximate values. The values can vary due to, for example, measurement or manufacturing tolerances (as will be understood by those of ordinary skill in the art) or other factors (as will be further understood by those of ordinary skill in the art). A measurement can vary, for example, by plus or minus 1 percent, plus or minus 5 percent, plus or minus 10 percent, plus or minus 15 percent, plus or minus 20 percent, plus or minus 1 to 5 percent, plus or minus 5 to 10 percent, or plus or minus 15 to 20 percent. Further, the measurements are for a specific implementation of the device, and other implementations can have different values, such as certain measurements, dimensions, both, or made longer to accommodate smaller hands or larger hands or to access tissue in a particular location of a patient's body.

For the specific implementations described, some specific values, ranges of values, and numbers are provided. These values indicate, for example, dimension, angles, ranges, frequencies, wavelengths, numbers, a relationship (e.g., relative value), and other quantities (e.g., numbers of sensors, sources, detectors, diodes, fiber optic cables, and so forth). Some measurements are for a specific implementation of the device, and other implementations can have different values, such as certain dimensions made larger for a larger-sized product or smaller for a smaller-sized product. The device may be made proportionally larger or smaller by adjusting relative measurements proportionally (e.g., maintaining the same or about the same ratio between different measurements). In various implementations, the values (or numbers or quantities) can be the same as the value given, about the same as the value given, at least or greater than the value given, or can be at most or less than the value given, or any combination of these. The values (or numbers or quantities) can also be within a range of any two values given or a range including the two values given. When a range is given, the range can also include any number within that range to any other number within that range.

The dimensions, for example, along an axis, a rotational orientation, or both are approximate values. The dimensions can be in values, directions, angles, or any combination of these dimensions. Dimensions, for example, of values in millimeters or centimeters, of directions along an axis, at an angular orientation relative to an axis, or of an angular orientation, are approximate values. The values, direction, and angles can vary due to, for example, measurement or manufacturing tolerances or other factors. A dimension can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, plus or minus 20 percent, plus or minus 0.1 to 0.2 percent, plus or minus 0.2 to 0.5 percent, plus or minus 0.5 to 1 percent, plus or minus 1 to 5 percent, plus or minus 5 to 10 percent, plus or minus 10 to 15 percent, or plus or minus 15 to 20 percent.

The shapes, for example, a geometric shape can be an approximate shape. The shapes can be in values, directions, angles, terms, or any combination of these shapes. The shapes can vary due to, for example, measurement or manufacturing tolerances or other factors. A shape can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, plus or minus 20 percent, plus or minus 0.1 to 0.2 percent, plus or minus 0.2 to 0.5 percent, plus or minus 0.5 to 1 percent, plus or minus 1 to 5 percent, plus or minus 5 to 10 percent, plus or minus 10 to 15 percent, or plus or minus 15 to 20 percent.

The orientations, for example, parallel, perpendicular, transverse, and angle are approximate values. The orientation can be in values, directions, angles, terms, or any combination of these orientations. Orientations, for example, of terms or angles, can be approximate orientations. The orientations vary due to, for example, measurement or manufacturing tolerances or other factors. An orientation can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, or plus or minus 20 percent. Terms, such as about, substantially, approximately, or other relative terms can include the described ranges as will be readily understood by those of ordinary skill in the art and can include ranges that will be understood by those of ordinary skill in the art.

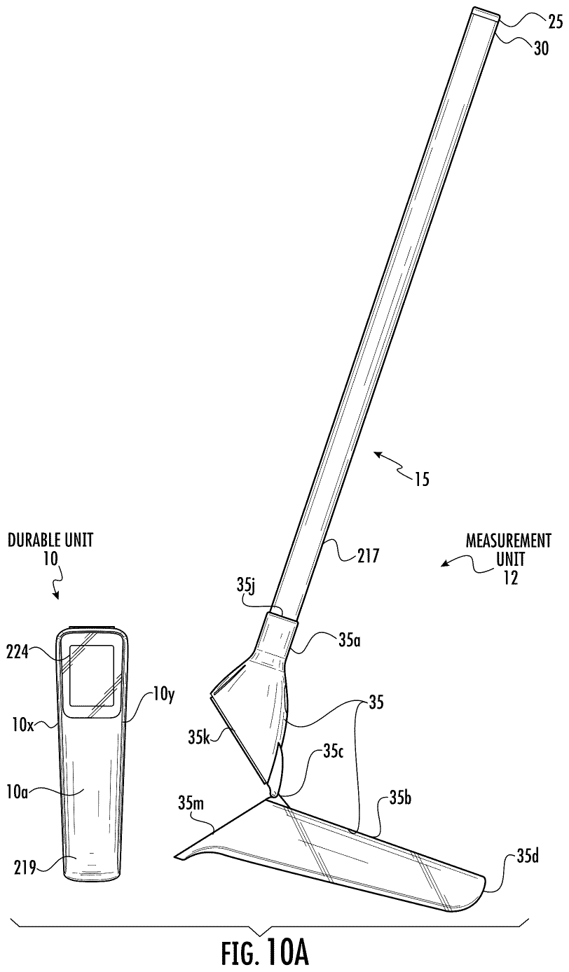

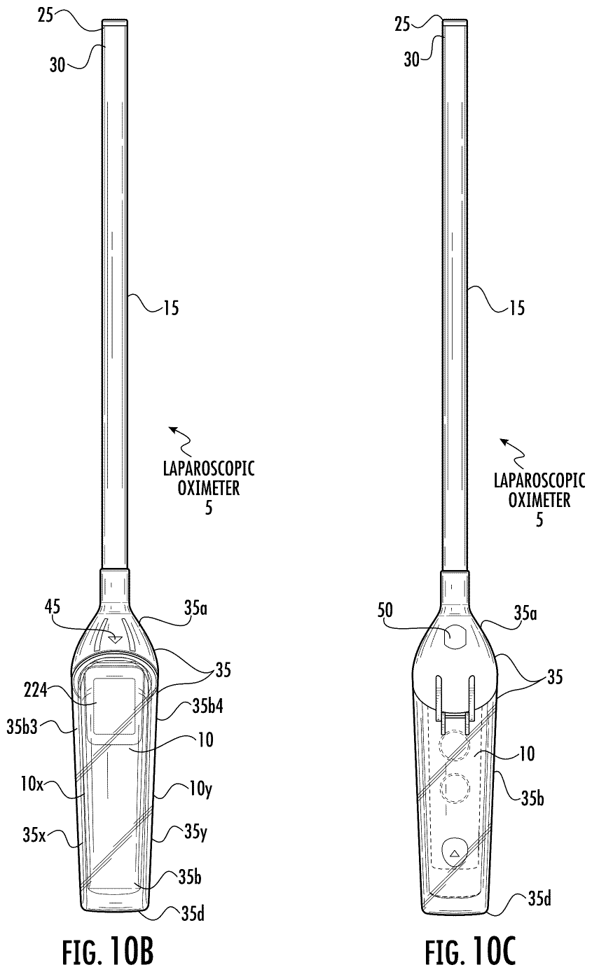

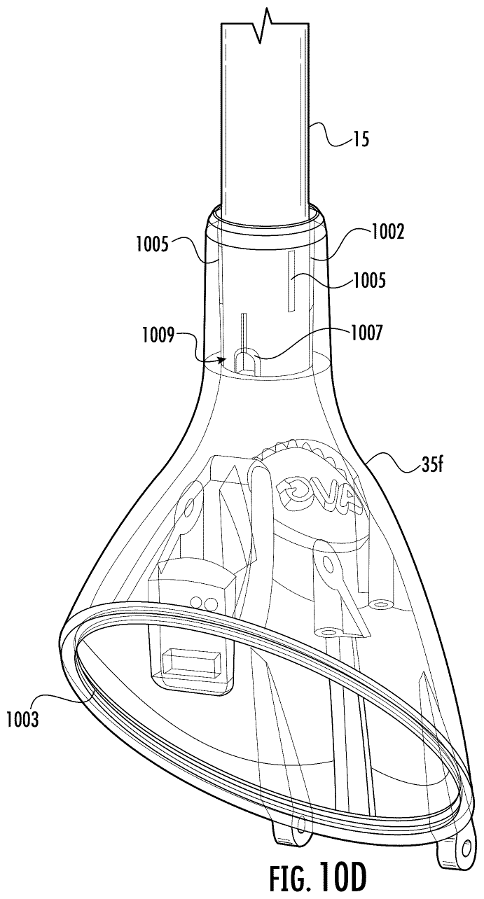

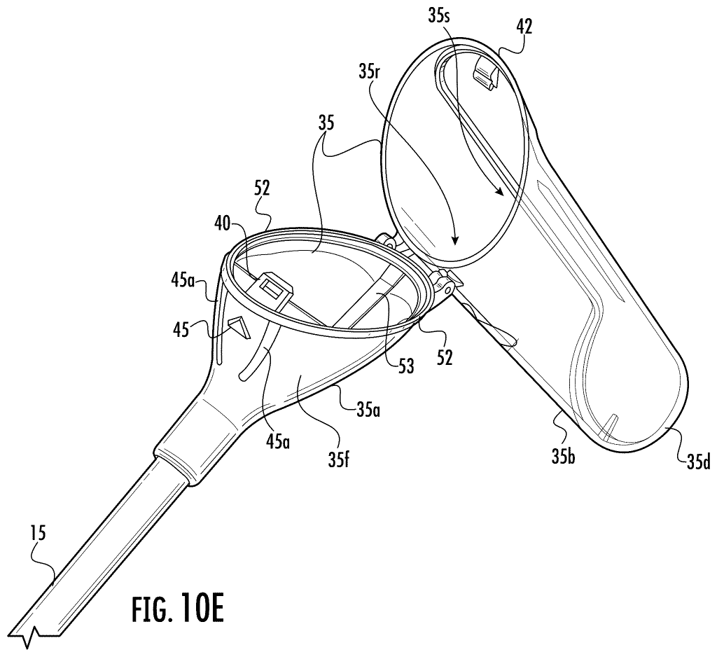

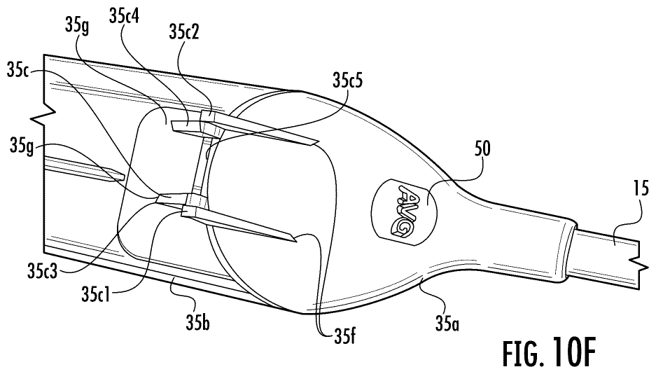

FIG. 10A shows durable unit 10 and measurement unit 12, in an implementation. Housing 35 is shown in an open configuration with lid 35a opened with respect to sheath 35b. The durable unit is shown outside of the sheath and separated from the measurement unit. The durable unit is insertable into the sheath and is electrically connectable to the laparoscopic tube via electrical connectors 20a and 22a.

Lid 35a and sheath 35b are coupled by a hinge 35c. The lid includes a first open end 35j and a second open end 35k. The first and second open ends of the lid are on opposite ends of the lid. An end of housing 217 of laparoscopic tube 15 is located in the first open end of the lid. The sheath includes a closed end 35d and an open end 35m. The closed end and open end of the sheath are on opposite ends of the sheath. Lid 35a can be rotated with respect to sheath 35b about hinge 35c so that the first open end of the lid and the open end of the sheath can contact and be separated from contact. The hinge allows the lid and sheath to be rotated with respect to each other so that the open ends contact and separate allowing for the housing 35 to be closed and opened. When the lid and sheath are open, the durable unit can be inserted into the sheath. When the lid and sheath are closed, the lid and sheath form a sealed space inside the housing where the durable unit can be enclosed in the sealed space. The closed and sealed housing prevents bodily fluids of a patient from contacting durable unit 10 and prevents contaminants on the durable unit from contacting patient tissue.

FIGS. 10B-10C show front and back views of oximeter device 5, in an implementation. The durable unit 10 is located in the closed housing 35 where the lid is closed onto the sheath of the housing. When the durable unit is in the closed housing, the durable unit extends from the lid 35a to the bottom 35d of the sheath 35b.

The lid includes a first button 45 and a second button 50. The first and second buttons are located on opposite sides of the lid, in an implementation. Button 45 is located on the front of the lid, and button 50 is located on the back of the lid. Button 45 may be pressed to open the lid from a closed configuration. Button 50 may be pressed to initiate average measurement generation by the processor of the durable unit. Average measurement generation is discussed further below.

FIG. 10D is a transparent view of the lid, in an implementation. The lid of the housing is rigidly connected to the laparoscopic tube. The connection between the lid and laparoscopic tube can be an adhesive connection (e.g., an epoxy connection) where the epoxy is located in a first opening 1002 of shell 35f of lid 35a. More specifically, the adhesive can be located between the inner side wall of the first opening and the outer sidewall of the laparoscopic tube where the end of the tube is located in the first opening. In an implementation, the epoxy can retract into opening 1002, leaving an open space at the end of opening 1002. Opening 10002 is oppositely located from a second opening 1003 of the lid.

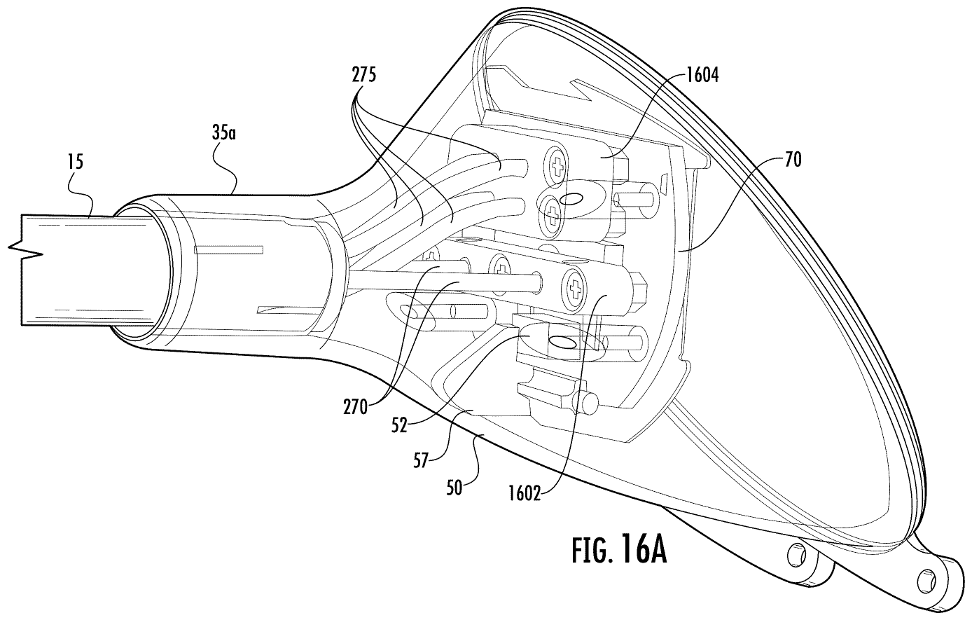

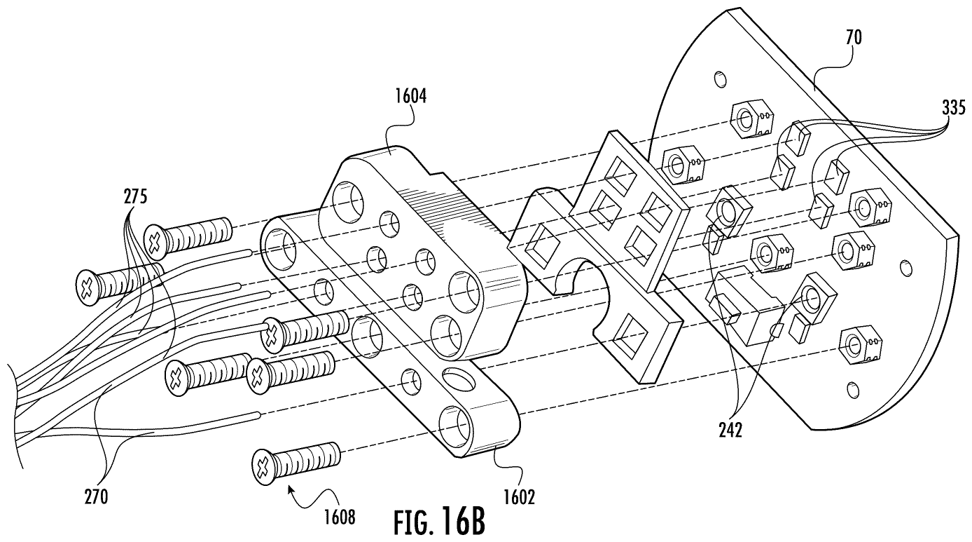

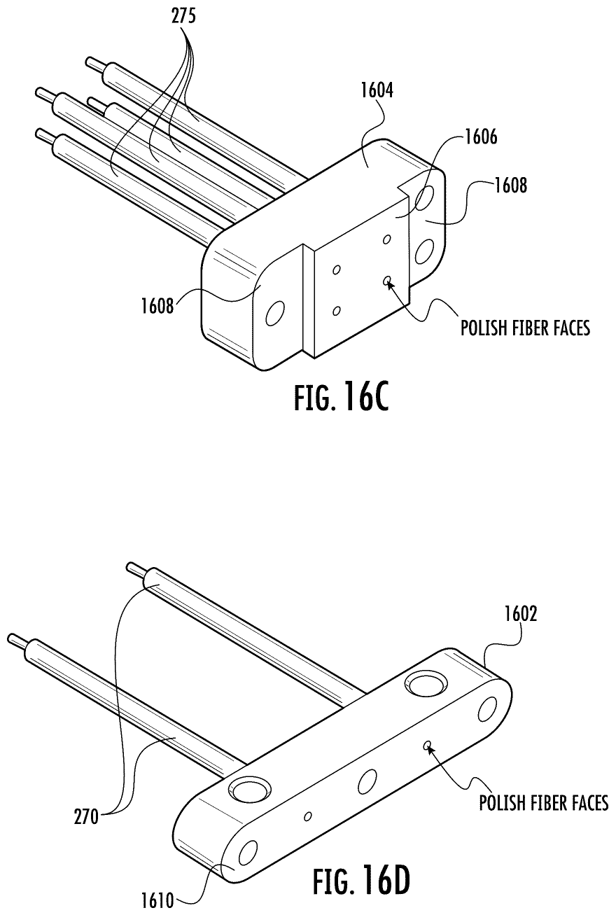

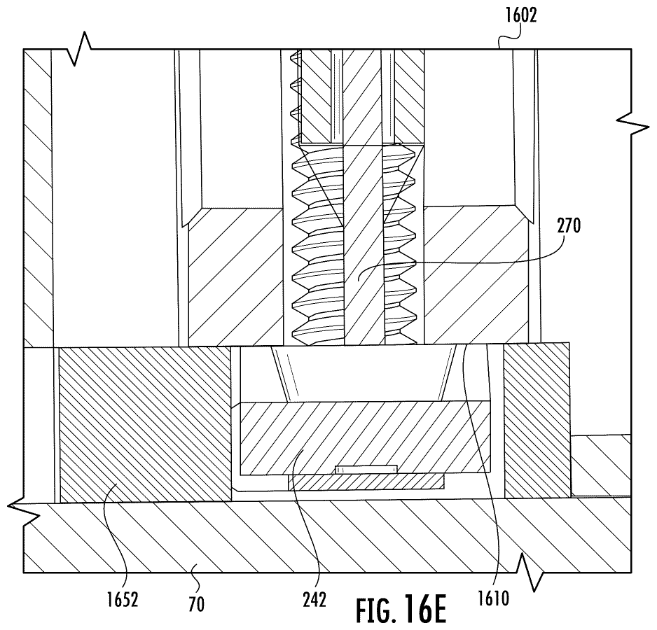

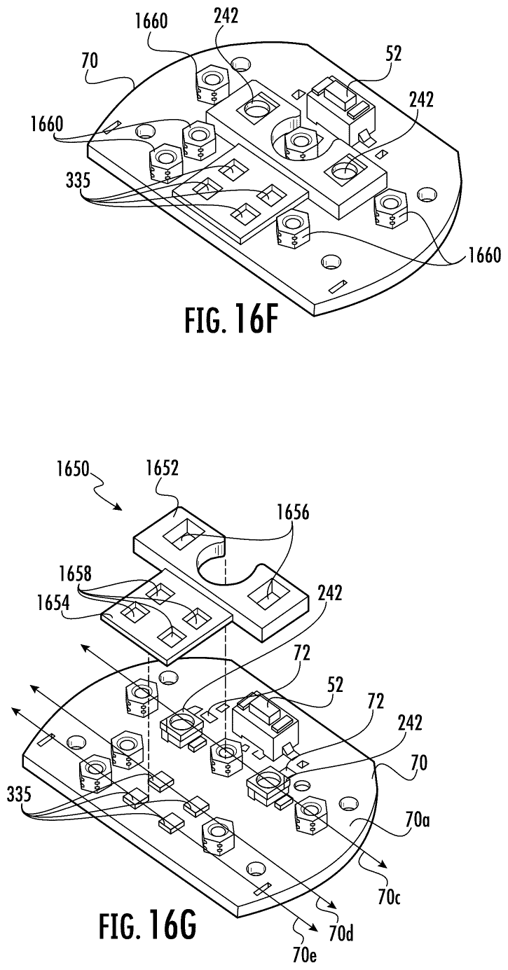

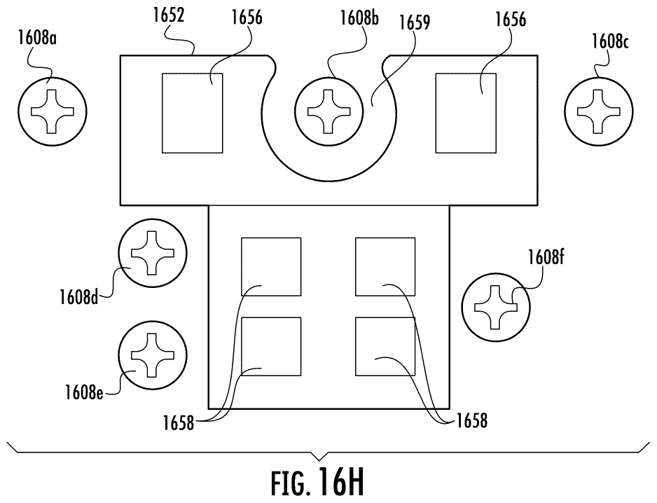

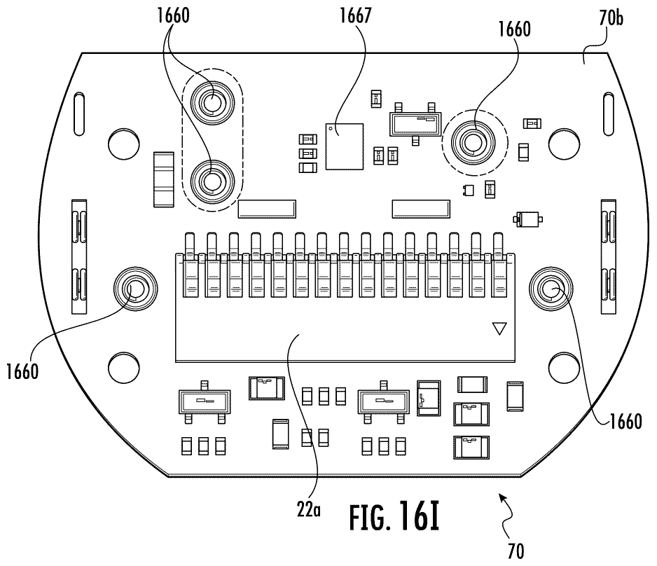

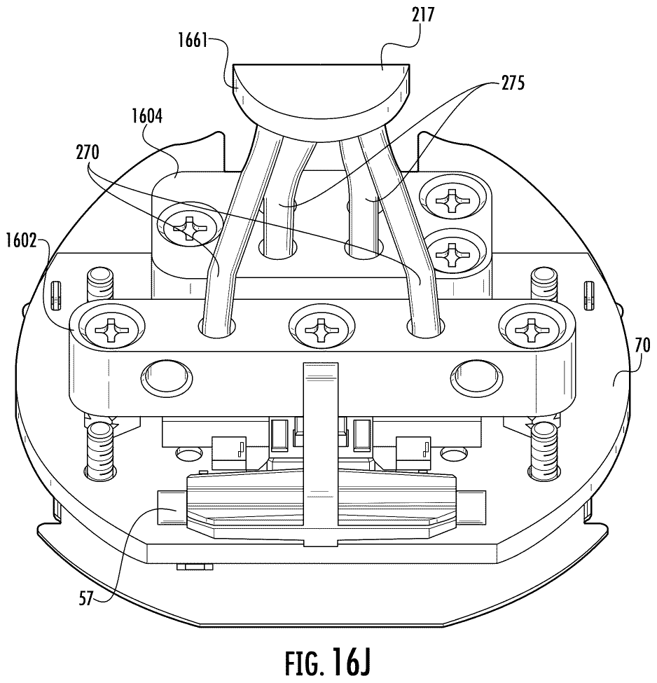

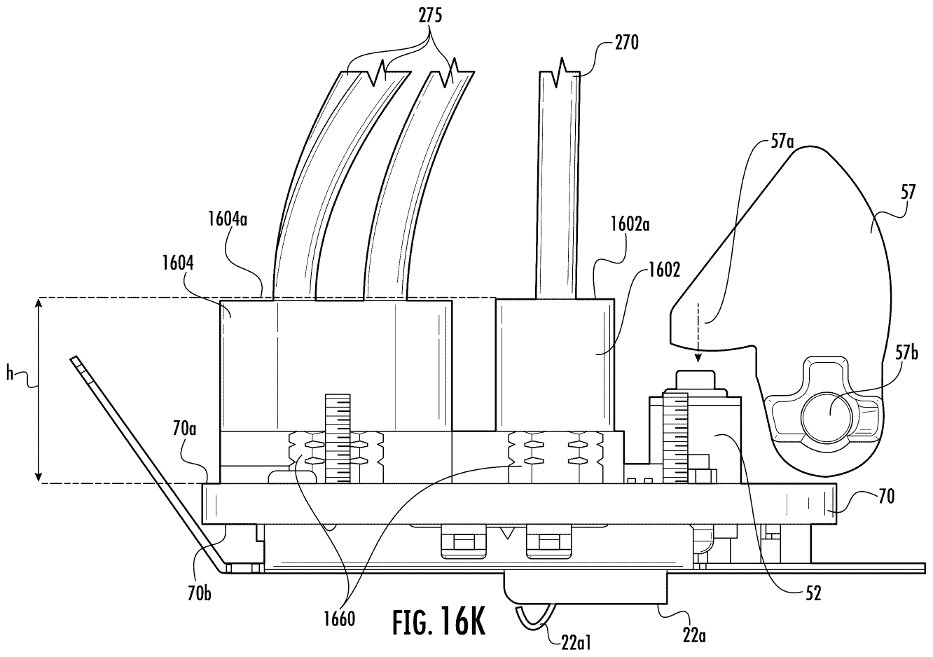

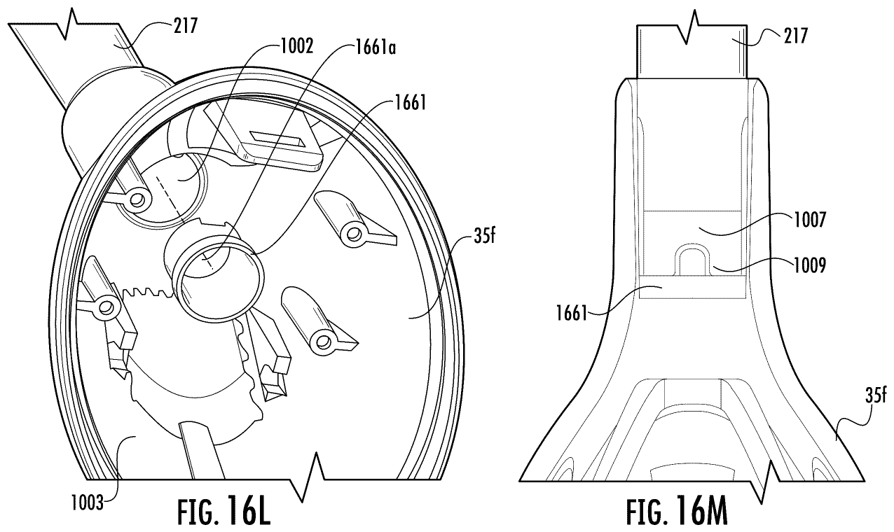

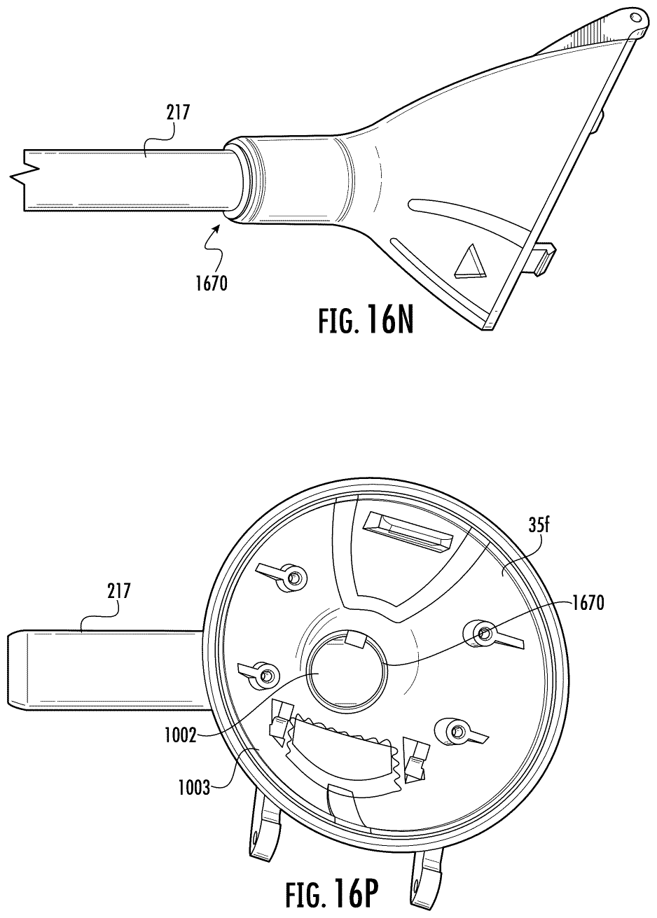

The connection between the lid and laparoscopic tube can be a mechanical connection. The mechanical connection can be a static friction connection, such as a press fit mechanism, where vertical fins 1005 located on an inside wall of the opening at the end of the lid connect with the laparoscopic tube when the tube is inserted in the opening to hold the tube in the opening. The connection between the lid and the laparoscopic tube can include an alignment mechanism, such as a notch (e.g., a notch on the lid or a notch on the laparoscopic tube 1007) that is configured to receive a tab (e.g., a tab on the lid 1009, a tab of a fiber protector 1661, which is shown in FIG. 16J-16L or a tab on the laparoscopic tube) when the laparoscopic tube is inserted in the opening.

The mechanical connection between the lid and the laparoscopic tube can be a threaded connection. The housing of the laparoscopic tube can include external threads on the end of the housing and the lid can include threads inside the end of the lid. The housing of the laparoscopic tube can include internal threads on the end of the housing and the lid can include external threads on the end of the housing.

The connection between the lid and the laparoscopic tube can be a welded connection (e.g., a plastic-welded connection). The connection between the tube and lid can include one or more of these connection types in any combination.

FIG. 10E shows a view of the housing in an open configuration and shows interior views of the lid 35a and sheath 35b. In an implementation, lid 35a includes a shell 35f wherein button 45 forms a portion of the shell. A first portion of a latch 40 forms a portion of the shell. In an implementation, button 45 and latch 40 are connected so that when button 45 is pressed, latch 40 moves inward towards the interior space in the lid. Latch 40 and button 45 may be integrally formed from the same material during the manufacture of the lid. Shell 35f, latch 40, and button 45 may be integrally formed from the same material during the manufacture of the lid. The material can be a relatively rigid plastic-type material, such as a first type of polymer. For example, the material can be acrylic, polycarbonate, HDPE, PVC, ABS, PTFE, HIPS, acetal, resin, or other types of plastic material.

The sheath 35b of the housing 35 can be formed of the same material or a different material from the shell 35f of the lid, such as the same or different types of polymers. Sheath 35b can be formed of acrylic, polycarbonate, HDPE, PVC, ABS, PTFE, HIPS, acetal, resin, or other types of plastic material. The lid can be opaque, whereas the sheath can have portions that are transparent, translucent, opaque, or a combination of these qualities.

In an implementation where one or both of the shell of the lid and the sheath are formed of a polycarbonate material, the polycarbonate can have one or more of the following properties. The polycarbonate, for example, can have a very high flow, which allows for injection molding. The polycarbonate can be biocompatible for oximetry applications, such as laparoscopic oximetry applications. In an implementation, the polycarbonate can be sterilized for healthcare applications. The polycarbonate can be exposed to ethylene oxide (EtO) gas in a high-flow environment for sterilization.

The polycarbonate's durometer hardness can be from about 75 to about 85 Shore D, and is about 80 Shore D, in an implementation. The tensile modulus of the polycarbonate is from about 2300 megapascals to about 2410 megapascals. The tensile strength of the polycarbonate is from about 60.0 megapascals to about 63.0 megapascals for yield strength, and from about 55.0 megapascals to about 58 megapascals for break strength.

Tensile elongation of the polycarbonate has a yield at about 5.7% to about 6.0%, and has a break elongation of from about 109% to about 111%, and is about 110%, in an implementation. Flexural modulus of the polycarbonate is from about 2300 megapascals to about 2410 megapascals. Flexural stress of the polycarbonate is from about 2300 megapascals to about 2410 megapascals.

The polycarbonate has a nominal transmittance through 2540 micrometers of material of about 87% to about 89%, and is about 88%, in an implementation. The polycarbonate can include dyes or particulate matter that lowers the transmittance. The polycarbonate has a nominal haze through 2540 micrometers of material of about less than 1%. The polycarbonate can include dyes or particulate matter that increases the haze.

In an implementation, the lid and shell are formed of different materials, for example, the lid is acrylonitrile butadiene styrene (ABS) and the shell is polycarbonate or the lid is polycarbonate and the shell is ABS. In an implementation, the lid and shell are formed of the same material, such as ABS or polycarbonate. In an implementation where one or both of the shell of the lid and the sheath are formed of ABS material, the ABS can have one or more of the following properties.

The ABS can have a healthcare certification for surgical instruments, such as laparoscopic oximeter 5. The ABS can have a durometer hardness from about 82 Shore D to about 100 Shore D, and can have a Rockwell R scale of about 110 to about 114, and in a specific implementation a R scale 112. The ABS can have a tensile stress at yield at 23 degrees Celsius of about 4.8194×10⁷ pascals (6990 pounds per square inch) to about 4.8332×10⁷ pascals (7010 pounds per square inch), and in a specific implementation is about 4.826310⁷ (7000 pounds per square inch). The ABS can have a tensile modulus of about 2.63000×10⁹ pascals to about 2.64000×10⁹ pascals, and in a specific implementation is about 2.620008×10⁹ pascals (380×10³ pounds per square inch).

The ABS can have a flexural strength at 23 degrees Celsius of about 75842340 pascals to about 75842350 pascals, and in a specific implementation is about 75842330 pascals (about 11000 pounds per square inch). The ABS can have a flexural modulus at 23 degrees Celsius of about 2.688945×10⁹ pascals to about 2.688970×10⁹ pascals, and in a specific implementation is about 2.688955×10⁹ pascals (e.g., about 90×10³ pounds per square inch). The ABS can have a density of about 1.03 grams per centimeter cubed to about 1.07 grams per centimeter cubed, and in a specific implementation is about 1.05 grams per centimeter cubed.

In an implementation, the lid includes elastomeric strips 45a that are located in cutouts formed in the shell 35f. The cutouts can be molded when the shell is molded. The elastomeric strips are connected to the sides of button 45 and the sides of shell 35f at the cutout.

Elastomeric strips 45a provide a seal between the sides of the button and the shell to prevent contaminants in the housing 35 (e.g., closed lid and sheath) from contacting a patient and to prevent contaminants outside of the housing, such as from a patient, from contacting a durable unit 10 sealed in the housing.

The elastomeric strips are flexible, can stretch, and return to a non-stretched configuration without deformation, which allows the button to be pressed while maintaining a seal of the button to the shell. The elastomeric strips can be a second type of polymer material that is a different type of polymer material than the first type of polymer material of the shell 35f. The second type of polymer material can have a higher modulus of elasticity (i.e., higher elasticity) than the first type of polymer material. The sheath of the housing can be a third type of polymer that is different from the first and second types of polymers of the lid and elastomeric strips.

In an implementation, the elastomeric material forming the elastomeric strips, buttons, and seals can have one or more of the following properties. The elastomeric material can form a good bond with polycarbonate and ABS.

The elastomeric material can have a durometer hardness at 23 degrees Celsius of about 55 Shore A to about 63 Shore A, and in a specific implementation is about 59 Shore A. The elastomeric material can have a specific gravity of about 0.898 grams per centimeter cubed.

The elastomeric material can have a tensile stress at 23 degrees Celsius of about 3.38 megapascals to about 3.40 megapascals, and in a specific implementation is about 3.39 megapascals. The elastomeric material can have a tensile strength at 23 degrees Celsius of about 4.49 megapascals to about 4.51 megapascals, and in a specific implementation is about 4.5 megapascals.

The elastomeric material can have a tensile elongation at 23 degrees Celsius of about 550 percent to about 570 percent, and in a specific implementation is about 560 percent. The elastomeric material can have a tear strength of about 31.3 kilonewtons per meter to about 31.7 kilonewtons per meter, and in a specific implementation is about 31.5 kilonewtons per meter.

Latch 40 includes a frame, where the frame may have a rectangular shape or another shape. Latch 40 includes an aperture formed in the frame, where the aperture can extend from a front of the frame to a back of the frame. When button 45 is pressed, latch 40 is moved inwards towards the inner space of the lid.

Sheath 35b includes a second portion of a latch 42. Latch 42 includes an extended arm and a tab extending from the arm at or near the end of the arm. Latch 42 can be integrally formed with sheath 35b during the manufacture of the sheath and latch. The latch and sheath can be formed of the same material.

The arm and tab both extend into the open space where the tab faces generally towards the bottom 35d of the sheath. When the lid and sheath are closed, the tab of latch portion 42 enters the aperture of the frame of latch portion 40 to hold the lid closed onto the sheath. Button 45 can be pressed to move the frame away from the arm and the tab to unlatch the latched first and second latch portions. In an alternative implementation, latch portion 40 includes an arm and tab, and latch portion 42 includes a frame and aperture where the tab of the latch enters the aperture of the frame to hold the lid closed on the sheath.

In an implementation, sheath 35b includes a button that is connected to a latch portion. In the implementation, the latch portion that is connected to shell 35f is not connected to a button of the shell.

In an implementation, the lid includes an elastomeric o-ring 52 that is located on a top edge of the lid and extends around the entirety of the top edge. The elastomeric o-ring is configured to deform towards the open space in the lid when button 45 is pressed and when latch 40 moves towards the open space inside the lid. In the implementation, sheath 35b does not include an o-ring attached to a top edge of the opening of the sheath.

The elastomeric o-ring can be located in a trench that is formed on the top edge of the lid or can be molded over the top edge of the lid. In an implementation, elastomeric o-ring 52 is integrally formed with elastomeric strips 45a from the same material. The elastomeric o-ring connects with the top edge of sheath 35b when the lid is closed onto the sheath. The elastomeric o-ring provides a seal between the top edges of the lid and the sheath of the housing when the lid is closed. The elastomeric o-ring prevents contaminants in the housing from contacting a patient and prevents contaminants of a patient (e.g., bodily fluids) from contacting a durable unit 10 sealed in the housing.

In an implementation, sheath 35b includes an elastomeric o-ring that is attached to a top edge of the opening of the sheath, and the lid does not include an elastomeric o-ring attached to a top edge of the opening of the lid.

In an implementation, lid 35a includes an elastomeric button 50 that is connected to shell 35f. Button 50 is further connected to a switch that is located on a PCB in the lid. The switch and PCB are described further below. Elastomeric button 50 is formed of an elastomeric material so that the button can be pressed and can return to its original shape after being released. Button 50 can retain a seal of the lid when pressed and released.

The lid includes an elastomeric strip 53 that connects with elastomeric button 50 and elastomeric o-ring 52. The elastomeric strips 45a, elastomeric button 50, elastomeric o-ring 52, and elastomeric strip 53 can be formed of the same material and can be integrally formed when the lid is manufactured. The elastomeric strips 45a, elastomeric button 50, elastomeric o-ring 52, and elastomeric strip 53 can be overmolded over shell 35f during manufacture after the lid is formed, such as by injection molding or over molding. The elastomeric material of elastomeric strips 45a, elastomeric button 50, elastomeric o-ring 52, and elastomeric strip 53 can be softer than the relatively rigid plastic of shell 35f. More specifically, the elastomeric material can have a high elasticity that is higher than the elasticity of the relatively rigid plastic type material that shell 35f is formed of.

FIG. 10F shows a view of the hinge 35c that hinge couples the lid 35a and the sheath 35b. Hinge 35c includes hinge posts 35cl and 35c2 formed on the lid and hinge posts 35c3 and 35c4 formed on the housing. Hinge posts 35c3-35c4 are located nearer to each other (i.e., inside) than hinge posts 35c3-35c4. Hinge posts 35cl and 35c3 can contact each other, and hinge posts 35c2 and 35c4 can contact each other. Hinge posts 35c3-35c4 are located nearer to each other (i.e., inside) than hinge posts 35c3-35c4.

The hinge includes a rod (e.g., a metal rod) 35c5 inserted through openings formed in each of the hinge posts. Portions of the rod adjacent to hinge posts 35c3 and 35c4 are crimped so that the hinge pin does not slide out from the hinge posts. The hinge rod can be smooth (e.g., polished) at the ends to limit the chance that the rod can snag and tear a surgical glove during use.

In an implementation, the center of mass of the oximeter 5 is located nearer to the hinge 35c than to the end 35d of the sheath. With the center of mass located nearer to the hinge than the end 35d, the oximeter can be balanced in a user hand where the user typically holds the oximeter. This location of the center of mass allows for precise placement of the sensor head onto tissue to be measured without the oximeter tending to tip forward or backward in a user's hand. In an embodiment, the center of mass of the oximeter is located in the between the ends 35f of the hinge portions coupled to the lid and ends 35g of the hinge portions couple to the sheath. In an embodiment, the center of mass of the oximeter is located between the ends of the hinge portions coupled to the lid. In an embodiment, the center of mass of the oximeter is located between the ends of the hinge portions coupled to the sheath. In an embodiment, the center of mass of the oximeter is located between the ends of the lid. Each of these locations for the center of mass are located where a user may typically hold the oximeter inhibiting the oximeter from tilting forward or backward in a user's hand during use allow for precise placement of the sensor head on tissue to be measured.

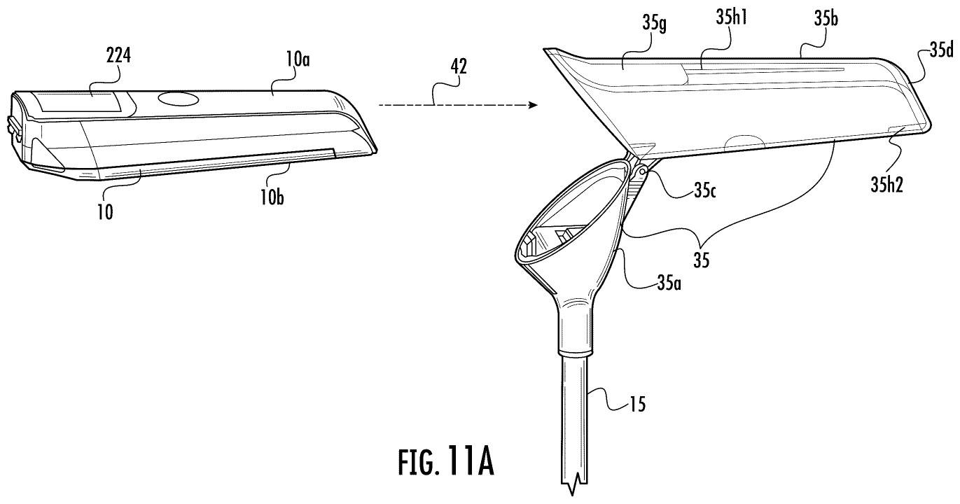

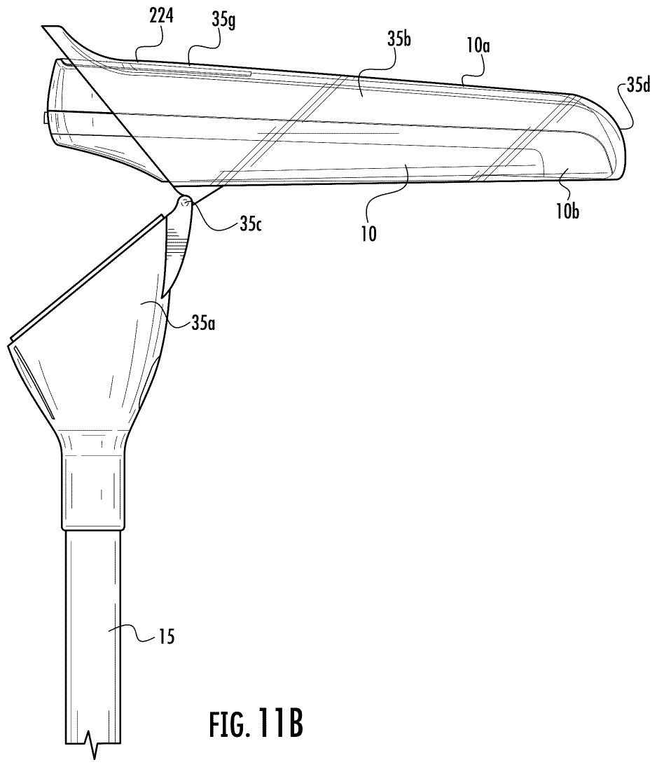

FIG. 11A shows durable unit 10, laparoscopic tube 10, and housing 35 in an implementation where the lid 35a and sheath 35b are open. The durable unit is oriented for insertion into sheath 35b. The surface of the shell 35b includes a window 35g. The window faces away from the laparoscopic tube 15 and lid 15a when the durable unit is oriented for insertion into the sheath. The display 224 of the durable unit faces the same direction as window 35g of the sheath when the durable unit is oriented for insertion into the sheath. Arrow 42 indicates the direction of insertion of the durable unit into the sheath. Window 35g is transparent, which allows for the display 224 to be viewed from outside of the sheath when the durable unit is located in the sheath. Other portions of sheath 35b, not including the window, can also be transparent, translucent, or opaque. In a specific implementation, the window of the sheath is transparent, while other portions of the sheath are translucent. The amount of translucence can vary, such as from 99 percent light transmittance to 0 percent (which would be opaque). The transparent portion can be a polished plastic or polymer surface, which would be relatively smooth. The translucent portion can be patterned or textured to provide an obscure plastic or polymer that distorts or obscures the view through it, providing a reduced view while still allowing some light to pass through. In a implementation, the translucent portion has surface texturing on an outside of the sheath surface.

Techniques to form the transparent and translucent portions of the sheath can include forming during an injection molding process. A further technique the transparent portion can be polished by chemically or mechanically, or a combination (e.g., abrasion). Typically the transparent portion would be smooth and untextured. The translucent portion can be done chemically or mechanically, or a combination (e.g., sandblasting) to achieve frosting, texturing, or other surface treatment.

The outer surface, the inner surface, or both of sheath 35b that does not include window 35g are textured, in an implementation. The textured surface of the outer surface of the sheath improves the grip of the sheath, in an implementation. The lid can be transparent, translucent, or opaque.

In an implementation, sheath 35b includes a raised rib 35h1 located on an inside surface of a wall of the sheath. The raised rib can be from about 0.5 millimeters tall to about 2 millimeters tall and from about 0.5 millimeters wide to about 3 millimeters wide. The raised rib can extend from an end of window 35g to a location that is a distance from the end 35d of the sheath. In an implementation, the raised rib does not extend to the window and there is a distance between window 35g and the end of the raised rib that is closest to the window. The raised rib can be from about 1 centimeter long to about 8 centimeters long. The raised rib is located on the opposite side of the sheath from the hinge 35c.

In an implementation, sheath 35b includes a raised rib 35h2 located on an inside surface of a wall of the sheath. The raised rib can be from about 0.5 millimeters tall to about 2 millimeters tall and from about 0.5 millimeters wide to about 3 millimeters wide. The raised rib can extend from an end 35d of the sheath to a location that is a distance from the hinge 35c. The raised rib can be from about 1 centimeter long to about 8 centimeters long. The raised rib is located on the opposite side of the sheath from window 35b and is located on the same side of the sheath as hinge 35c.

FIG. 11B shows the durable unit 10 located in sheath 35b of housing 35, in an implementation. When the lid is open, as shown in FIG. 11B, a portion of the durable unit is located outside of the sheath.

Further, when the durable unit 10 is located in sheath 35b, the top surface 10a and the bottom surface 10b of the durable unit contact ribs 35h1-35h2. And, the bottom 10c of the durable unit contacts the bottom 35d of the sheath. Other portions of the housing of the durable unit contact other internal surfaces of the sheath when the durable unit is located in the sheath. Contact of the durable unit with the raised ribs, the bottom of the housing, and other internal surfaces of the housing establishes the orientation and the location of the durable unit in the sheath. The established orientation and location of the durable unit in the sheath allows for the electrical contacts of electrical connectors 20a and 22a of the durable unit and the lid to align and contact when the lid and sheath are closed.

FIG. 11C shows the durable unit 10 located in closed housing 35 in an implementation where the lid and sheath are rotated into contact. When the housing is closed, an open space that is sealed by seal 55 is created that seals the durable unit in the sheath. When the durable unit is in the space in the closed housing 35, the lid 35a and sheath 35b press electrical connectors 20a and 22a together, forming the fully functional laparoscopic oximeter 5. Specifically, the electrical connectors 20a and 22a are electrically and mechanically connected when the lid is rotated into contact with the sheath. That is, the electrical connectors are rotated into contact when the lid and sheath are rotated into contact. The view shown in FIG. 11C provides an understanding of the orientation of the durable unit and lid when the electrical connectors 20a and 22a are connected.

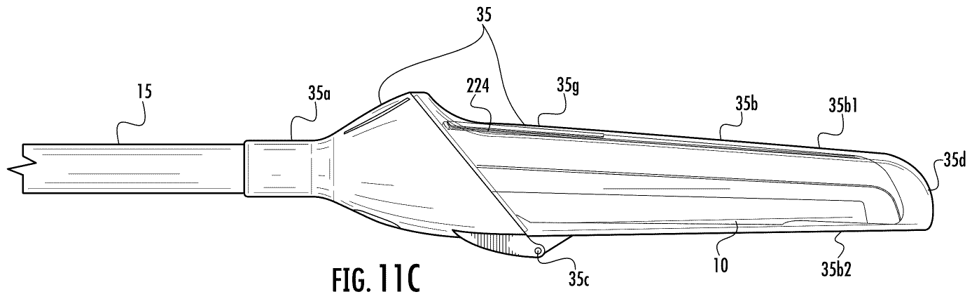

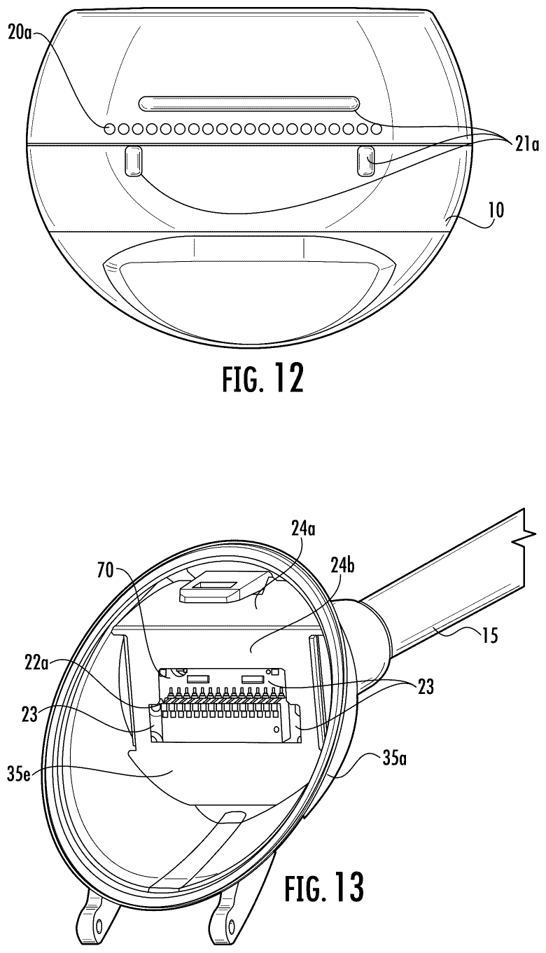

FIG. 12 shows an end view of the top of durable unit 10, and FIG. 13 shows an end view of the lid 35a. The end portion of durable unit 10 includes the electrical connector 20a, which includes a number of electrical contacts. The number of electrical contacts can be from 4 to about 40 (e.g., 20 in a specific implementation). The electrical contacts can be fixed mechanical contacts or can be spring contacts. The electrical contacts can be gold-plated contacts. Further, the contacts can be linearly arranged or arranged in another configuration.

The interior portion of lib 35a includes a PCB 70 on which electrical connector 22a is located. Electrical connector 22a can include a number of electrical contacts. The number of electrical contacts can be from 4 to about 40 (e.g., 15 in a specific implementation). The number of electric contacts of electrical connectors 20a and 22a is the same. The electrical contact can be fixed mechanical contacts or can be spring contacts. The electrical contacts can be gold-plated contacts. The electrical contacts can be linearly aligned or arranged in another configuration.

The electrical contacts of connectors 20a and 22a have complementary arrangements so that the contacts connect when the durable unit is located in the housing and when the lid and sheath are closed. The electrical contacts include ground contacts, power contacts, and signal contacts. For example, the durable unit can be configured to supply power and ground to the PCB of the lid from the power supply of the durable unit. For example, power and ground from the durable unit can be supplied to various circuits located on the PCB of the lid, such as NCF device 212, light engine 240, ADC 20d, amplifiers 256, and photodetectors 259.

In an implementation, the top of durable unit 10 includes a number of ribs 21a, such as three ribs. One rib can have a longitudinal axis that is parallel to an axis on which the electrical contacts are aligned. Two ribs can be perpendicular to the axis on which the electrical contacts are aligned. Each rib can be from about 2 millimeters long to about 30 millimeters long and from about 1 millimeter wide to about 3 millimeters wide.

The lid includes walls 24a and 24b, which can be perpendicular. The walls can be formed of metal or metalized plastic. Wall 24a includes an aperture formed in the walls and extends from a first side of the wall to a second side of the wall to create an opening. In an implementation, a portion of the PCB 70 of the lid is exposed by the aperture. Electrical contact 22a mounted on PCB 70 extends through the aperture. The aperture includes extended open spaces 23 along at least three sides of the electrical contact. When the durable unit is located in the sheath and the lid is being closed, ribs 21a of the durable unit enter the open spaces 23 and may contact the edges of wall 24a to guide the alignment of the electrical contacts of electrical connectors 20a and 22a. In one implementation, ribs 21a do not contact the PCB through open spaces 23. In one implementation, ribs 21a contact the PCB through open spaces 23. The ribs can aid in setting the pressure between the electrical contacts of electrical connectors 20a and 22a. In an implementation, wall 24a is metal and can be gold plated.

The laparoscopic tube includes source waveguides 270, detector waveguides 275, and a sensor head 25 located at a tip 30 of the laparoscopic tube. Portions of the source and detector waveguides (also sometimes referred to as optical conductors) can extend into the interior space of the lid that contains the electrical coupler 22a, light engine 240, ADC 20d, amplifiers 256, and photodetectors 259.

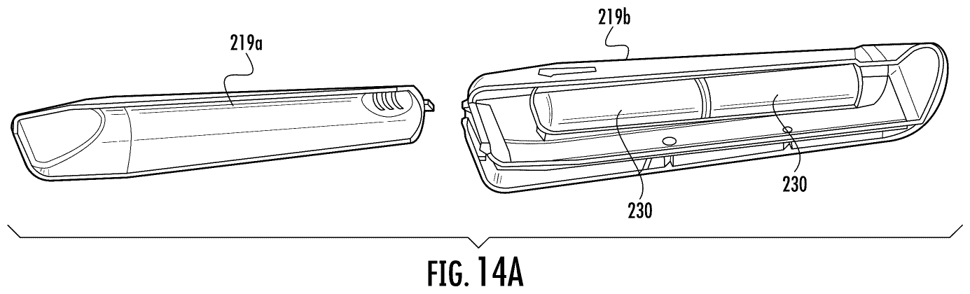

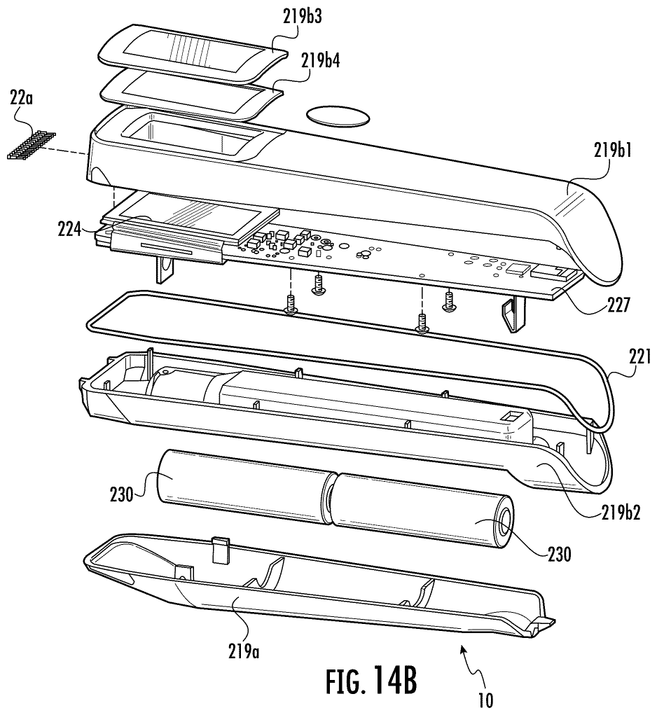

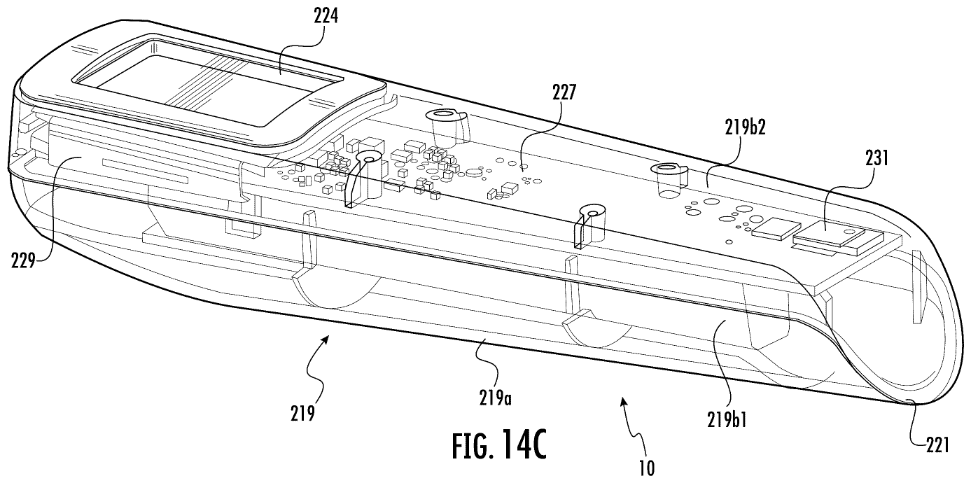

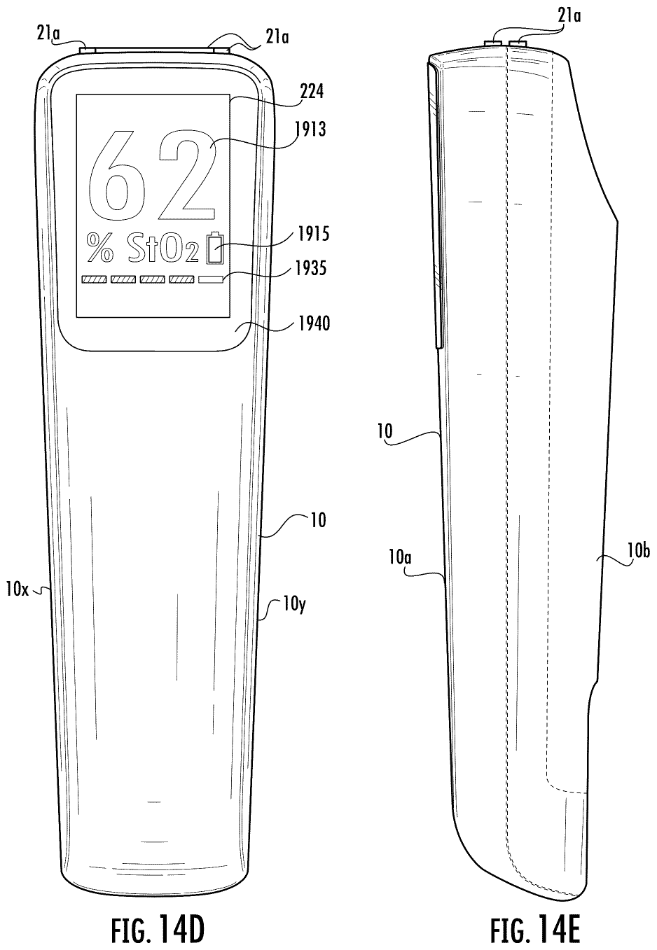

FIG. 14A shows a view of durable unit 10 with the battery cover 219a removed from housing 219b. The battery cover can be slid onto the housing and latched to the housing. The batteries 230 (e.g., two AA or two AAA) can be replaced when the battery cover is removed.

The battery cover 219a and housing 219b can be formed of relatively rigid plastic type materials. For example, the battery cover and housing can be acrylic, polycarbonate, high-density polyethylene (HDPE), polyvinyl chloride (PVC), acrylonitrile butadiene styrene (ABS), polytetrafluoroethylene (PTFE), high-impact polystyrene (HIPS), acetal, resin, or other types of plastic material.

In an implementation where the battery cover and housing are ABS, the ABS material can have the following characteristics. The ABS can have a healthcare certification for surgical instruments, such as laparoscopic oximeter 5. The ABS can have a durometer hardness from about 82 Shore D to about 100 Shore D, and can have a Rockwell R scale of about 110 to about 114, and in a specific implementation a R scale 112. The ABS can have a tensile stress at yield at 23 degrees Celsius of about 4.8194×10⁷ pascals (6990 pounds per square inch) to about 4.8332×10⁷ pascals (7010 pounds per square inch), and in a specific implementation is about 4.8263107 (7000 pounds per square inch). The ABS can have a tensile modulus of about 2.63000×10⁹ pascals to about 2.64000×10⁹ pascals, and in a specific implementation is about 2.620008×10⁹ pascals (380×10³ pounds per square inch).

The ABS can have a flexural strength at 23 degrees Celsius of about 75842340 pascals to about 75842350 pascals, and in a specific implementation is about 75842330 pascals (about 11000 pounds per square inch). The ABS can have a flexural modulus at 23 degrees Celsius of about 2.688945×10⁹ pascals to about 2.688970×10⁹ pascals, and in a specific implementation is about 2.688955×10⁹ pascals (e.g., about 90×10³ pounds per square inch). The ABS can have a density of about 1.03 grams per centimeter cubed to about 1.07 grams per centimeter cubed, and in a specific implementation is about 1.05 grams per centimeter cubed.

FIG. 14B shows an exploded view of durable unit 10, in an implementation. The housing 219b includes a top housing portion 219b1 and a bottom housing portion 219b2. A seal 221 is located between the top and bottom housing portions when the top and bottom housing portions are connected. The top and bottom housing portions can be adhered together by the double stick tape or by another adhesive, such as epoxy, fastened together, snap fit, plastic welded, or a combination of these connection types.

In an implementation, the seal material has the following properties. The seal material is a 0.10 millimeter to 0.4 millimeter thick (about 0.20 millimeters in a specific embodiment), black, double coated acrylic foam tape with a polyethylene terephthalate (PET) liner. The adhesive on both sides of the seal can bond the top and bottom housing 219a-219b together. The density of the foam tape ASTM D3574 is about 720 kilogram per meter cubed with a liner thickness of about 0.06 millimeters to 0.1 millimeters (e.g., about 0.08 millimeters in a specific embodiment. Tolerances can be about plus or minus about 15 percent. The water vapor transmission can be ASTM F1249 at 38 degrees Celsius with 100 percent relative humidity. The seal material can have a shear modulus of about 5952 pascals. The seal material can have an overlap shear strength ASTM D1002, ISO 4587 of about 690 kilopascals.

A window 219b3 and a seal 219b4 are connected to top housing portion 219b1. The seal seals the window to the housing above display 224. The seal, window, or both can be connected to the top housing portion via adhesive (e.g., epoxy), fastened together, snap fit, plastic welded, or a combination of these connection types.

The top and bottom housing portions 219b1-219b2 house a PCB 227. Various circuits are mounted on the PCB, including the electrical coupler 22a, processor 220, accelerometer 227, memory 222, display 224, and power source 235.

FIG. 14C shows a transparent view of durable unit 10, in an implementation. PCB 227 is located in the upper half of housing 219. The PCB extends under display 224. Shielding 229 is located at a first end of the PCB near display 224. The shielding can be a metal shield. In an implementation, a first transmitter circuit 231 (e.g., a Bluetooth circuit) is located at a second end of the PCB, which is oppositely located from the first end of the PCB. In an implementation, the processor 220 and the first transmitter circuit 231 are combined in a mixed single IC.

FIG. 14D shows a front view of durable unit 10, in an implementation. The left side 10x and the right side 10y of the durable unit are angled with respect to each other such that the durable unit tapers from a top of the durable unit to a bottom of the durable unit. An angle between the left and right sides is from about 2 degrees to about 6 degrees. In a specific implementation, the angle between the left and right sides of the durable unit is about 4 degrees.

The lift side 35b3 of the sheath 35b and the right side 35b4 of the sheath are angled with respect to each other similarly to the left and right sides of the durable unit. For example, an angle between the left and right sides of the sheath is from about 2 degrees to about 6 degrees. In a specific implementation, the angle between the left and right sides of the sheath is about 4 degrees.

When the laparoscopic oximeter measures oxygen saturation for patient tissue, the durable unit 10 can display oxygen saturation values 1913 on the display of the oximeter as shown in FIG. 14D. The oximeter can display a numerical value of the percentage of oxygen saturation, a bar level indication of the oxygen saturation value, a graph of oxygen saturation values over time, or any combination of these indicators. The oximeter can display a battery level indicator 1915 that indicates an amount of charge remaining in the battery. The battery level indicator can be a bar level indicator with a number of bars displayed in a first color to indicate a charge state. For example, the bar level indicator may include five bars. If the charge state of the batteries is 80 percent, then four of the five bars may be displayed as green to indicate an 80 percent charge state and the fifth bar may be displayed as another color, such as black to indicate a 20 percent discharge state of the batteries.

This display of the durable unit can display a contact indicator 1935 that that indicates the quality of contact between patient tissue and the sensor head of the oximeter. The contact indicator can be a bar level indicator with a number of bars displayed in a first color to indicate a quality of contact. For example, the bar level indicator may include five bars. The higher number of bars (such as 5 bars) displayed indicate a higher quality of contact with patient tissue and the sensor head. In contrast, a lower number of bars (such as 1 bar) displayed indicates a lower quality of contact between patient tissue and the sensor head.

The contact indicator can indicate a quality of an oximeter measurement. For example, if a quality of contact between the sensor head and tissue is low, then the quality of an oximetry value (e.g., oxygen saturation value) may be low. Alternatively, if a quality of contact between the sensor head and tissue is high, then the quality of an oximetry value (e.g., oxygen saturation value) may be low. Quality information displayed by the oximeter may be adjusted if the oximeter is moved by a user of shakes during tissue contact. In an embodiment, the accelerometer can be used by the oximeter to adjust quality information calculated by processor and displayed on the display.

In an implementation, the processor operates with the accelerometer to scale, modify, qualify, characterize, or any combination of these operations on quality information of a measurement or contact. For example, the accelerometer can detect whether the oximeter shakes in a user's hand while oximetry measurements are made. Shaking can angle the sensor head with respect to tissue being measured, which can raise or lower the quality of an oximetry reading. The shaking has a frequency. The processor can use movement information generated by the accelerometer in response to the movement of the oximeter to adjust the quality information. The processor can adjust quality information based on the frequency of the shaking. For example, the adjustment to the quality information can be adjusted proportionally to the frequency of the shaking. The quality information can be adjusted by the processor based on the length (e.g., the average length, maximum length, minimum length, or another length) of movement from the shaking.

In an implementation, the accelerometer, the detector structures using light collected by the detector structures, or other elements of the oximeter, measure the angular orientation of the sensor head respect to tissue being measured. The processor can use the angular orientation information to adjust the quality information. The processor can adjust the quality information metric based on the angle of tilt detected by the accelerometer relative to the tissue. For example, the angle is used for a proportional (e.g., multiplier) adjustment to the quality information.

The quality information can be adjusted based on a length of movement that the oximeter device has moved relative to the tissue (away from the tissue, across the tissue, or a combination of these movement lengths). The processor can adjust the quality information based on the length of the movement. For example, the length is used for a proportional (e.g., multiplier) adjustment to the quality information.

The quality information can be adjusted based on the direction of movement. A first correction can be applied for movement across the tissue (x-direction, y-direction, or both). A second correction can be applied for movement away from the tissue (z-direction). A third correction can be applied for movement toward the tissue (minus z-direction). The levels of the first, second, and third correction can be different. For example, the levels of correction may be different for the same lengths of movement across the tissue, upward from the tissue, and towards the tissue.

In an implementation, the accelerometer detects movement that persists for about 200 to about 400 milliseconds. In an implementation, the accelerometer detects movement if the movement persists for about 320 milliseconds or longer. And any movement below the acceleration of the earth's gravity is detected (e.g., above about 9.8 meters/second squared).

In an implementation, the processor does not recognize movement of the oximeter if the signal generated by the accelerometer is below a first threshold value (e.g., a first threshold voltage or a first threshold current). The processor may apply a first correction to the quality information for accelerometer output values (e.g., output voltages) above the first threshold value and below a second threshold value.

The processor may apply a second correction to the quality information for accelerometer output values (e.g., output voltages) above the second threshold value and below a third threshold value. The first and second corrections are different corrections where the second correction is larger than the first correction. The processor may apply a third correction to the quality information for accelerometer output values (e.g., output voltages) above the third threshold value and below a fourth threshold value. The second and third corrections are different corrections where the third correction is larger than the second correction. The processor may apply a fourth correction to the quality information for accelerometer output values (e.g., output voltages) above the fourth threshold value and below a fifth threshold value. The third and fourth corrections are different corrections where the fourth correction is larger than the third correction. The device may use additional threshold levels for an increasing amount of corrections, such a sixth, a seventh, an eighth, a ninth, or higher number of threshold levels.

In an embodiment, the accelerometer outputs information coordinate information for movement in a coordinate system, such as the Cartesian coordinate system. The output information may stored in an accelerometer register and transmitted to the processor for processing. The output information distance information for the distance the system unit has moved along one of the coordinate axes (e.g., Cartesian, polar, cylindrical, spherical, or others). The coordinate information output from the accelerometer is digital information.

In an implementation, processor adjusts the quality information based on a number of guard bands that are determined based on the information output by the accelerometer. There may be two or more guard bands that the processor determines based on the accelerometer output.

The number of bars displayed in the quality indicator adjusted based on the quality of contact of the sensor head and any adjustment based on the one or more described detectors on the accelerometer. In an implementation, the quality indicator includes a numerical value that is displayed on the display.

Information displayed on the display can include an indicator that the lid of the sheath has become opened, such as while the oximeter is in use. The durable unit can determine that the lid has opened when electrical contact becomes open between the one or more electrical contacts of the durable unit and one or more electrical contacts of the lid become open. The information displayed on the display can include information to such the lid or to replace the measurement unit with a different measurement unit.

FIG. 14E shows a side view of the durable unit 10, in an implementation. The front side 10a and the back side 10b of the durable unit are angled with respect to each other such that the durable unit tapers from a top of the durable unit to a bottom of the durable unit. An angle between the left and right sides can be from about 2 degrees to about 6 degrees. In a specific implementation, the angle between the left and right sides of the durable unit is about 4 degrees.

The front side 35b1 of the sheath 35b and the back side 35b2 of the sheath are angled with respect to each other similarly to the front and back sides of the durable unit. For example, an angle between the front and back sides of the sheath is from about 2 degrees to about 6 degrees. In a specific implementation, the angle between the front and back sides of the sheath is about 4 degrees.

In an implementation, display 224 is framed by a bezel 1940. The bezel can have a color (such as white) that contrasts with a color of the display when powered on (such as black) so that the information displayed on the display is easily visible to a user of the oximeter.

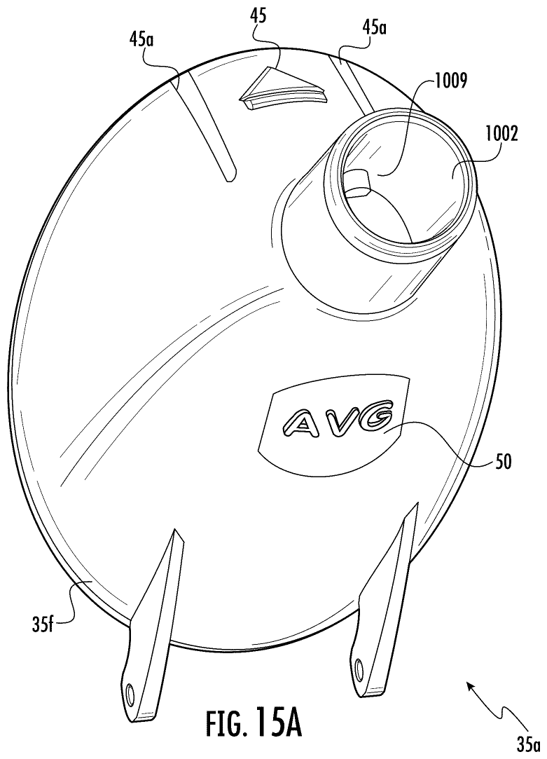

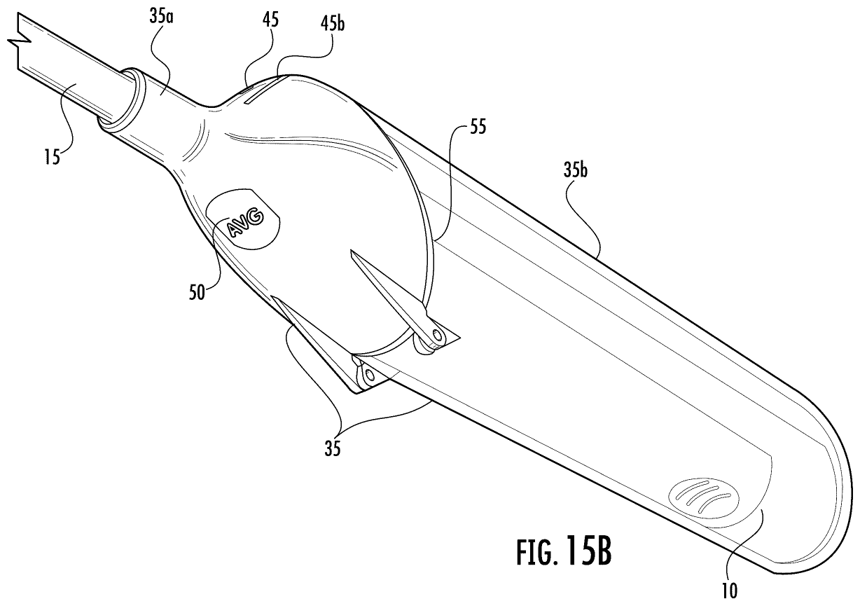

FIG. 15A shows a perspective view of lid 35a. Tab 1009, described above, is located in opening 1002 of the lid. The tab is located on the surface of the inner sidewall of opening 2001. FIG. 15a further shows shell 35f and button 45 integrally formed with the shell. The lid includes the elastomeric strips 45a that couple the side of button 45 to shell 35f. As described briefly above, the elastomeric strips are flexible and allow the button to flex at the end where the button is attached to the shell while maintaining a seal between the button and shell. The elastomeric strips allow the button to be pressed and can retain a seal of the housing. Button 45 is coupled to tab 40, and when button 45 is pressed, tab 40 is pressed so that the lid can be opened. Lid 35a includes button 50 that is coupled to a switch 52 on the PCB 70, shown in FIG. 16a and described further below. The switch is described further below.

FIG. 15B shows a view of the durable unit 10 located in the closed housing 35. The sheath 35b can be translucent so that the durable unit can be seen from an exterior of the sheath. The housing includes the elastomeric seal 55 that is located between the edges of the lid and the sheath when the lid is closed to seal the housing so that patient fluids and contaminants cannot enter the housing to contact the durable unit.

FIG. 16A shows a transparent view of the lid 35a, in an implementation. The interior space of the lid is shown in the transparent view. FIG. 16B shows an exploded view of components housed in the interior space of the lid, in an implementation. The lid includes a first guide block 1602 and a second guide block 1604. The guide blocks are connected to PCB 70. The guide blocks can be adhered to the PCB (e.g., epoxied), fastened to the PCB (e.g., fastened by screws 1608), or welded (e.g., plastic welded) to the PCB, or a combination of these fastening techniques. Guide block 1602 includes a number of apertures (e.g., 2 apertures) in which the ends of the source waveguides 270 are located. Guide block 1604 includes a number of apertures (e.g., 4 apertures) in which the ends of the detector waveguides 275 are located.

Guide block 1602 holds the ends of the source waveguides 270 in place relative to PCB 70 and relative to LEDs 242 (or laser diodes in an implementation) of light engine 240 located on the PCB. Guide block 1602 allows for a stable optical coupling of the source waveguides to the LEDs. The stable coupling allows light emitted from the LEDs to enter the source waveguide without variation in intensity because the ends of the waveguide cannot move with respect to the LEDs.

Guide block 1604 holds the ends of the detector waveguides 275 in place relative to PCB 70 and relative to photodetectors 335 located on the PCB. Guide block 1604 allows for a stable optical coupling of the detector waveguides to the photodetectors. The stable coupling allows light emitted from the detector waveguides to enter the photodetector without variation in intensity because the ends of the waveguide cannot move with respect to the photodetectors.

FIGS. 16C-16D show back views of guide blocks 1602 and 1604, in an implementation. The tips of the source waveguides and detector waveguides are polished and can be flush with the back surface of the guide blocks or can extend from the guide blocks. Polishing the tips of the source waveguides allows for improved transmission of light from the LEDs into the waveguides without reflection of light from the tips. Polishing the tips of detector waveguides allows for improved transmission of light from the waveguides into the photodetectors without reflection of light back into the waveguides.

FIG. 16E shows a cross-sectional view of PCB 70 and devices mounted on the PCB, in an implementation. As described above with respect to FIG. 16D, the end of source waveguide 270 is polished flush with the bottom surface 1610 of guide block 1602. In an implementation, the end of source waveguide 270 contacts LED 242. The contact can be a flush contact between the source waveguide and the LED. In an implementation, a gap is located between the end of the source waveguide 270 and LED 242. In an implementation, an optical element, such as a lens, is located between the end of the source waveguide and the LED. In an implementation, fluid, such as gel, is located between the end of the source waveguide and the LED. The top of the baffle can be above the top surface of the LED to allow for the gap, optical components, or both to be located between the end of the LED and the end of the source waveguide.

The baffle 1652 contacts the bottom 1610 of the guide block to block stray light from the LED from reaching the other waveguides and from reaching any of the photodetectors. The other source waveguide not shown in FIG. 16e is configured the same as the source waveguide shown in this figure.

As described above with respect to FIG. 16C, the end of the detector waveguides are polished flush with the bottom of guide block 1604. In an implementation, the end of each detector waveguide 275 contacts a photodetector 335. The contact can be a flush contact between the detector waveguide and the photodetector. In an implementation, a gap is located between the end of each of the photodetectors and the detector waveguides. In an implementation, an optical element, such as a lens, is located between the end of each detector waveguide and each photodetector. In an implementation, fluid, such as gel, is located between the end of each detector waveguide and each photodetector. The top of the baffle can be above the top surface of the photodetector to allow for the gap, optical components, or both to be located between the ends of the photodetectors and the ends of the detector waveguides.

In an implementation, the source and detector waveguides are plastic fibers. The source and detector waveguides are high numerical aperture waveguides and have relatively large diameters, as is common for plastic fibers. The fiber material, acceptance angles, or both can be used to transmit higher numbers of photons compared to smaller-diameter optical fibers and lower numerical aperture optical fibers.

In an implementation, each detector waveguide transmits light collected from tissue to the photodetectors (e.g., photodiode detectors), which generate electrical signals from the light. The diameters of the detector waveguides are smaller than the diameters of the photodetectors, so that essentially all of the light transmitted through the detector waveguides is directed onto the photodetectors. The fixed fraction of light transmitted from the detector waveguides to the photodetectors provides improved accuracy of oximetry measurements more than the absolute magnitude of transmitted light from the detector waveguides to the photodetectors. However, transmitting essentially all of the light transmitted from the detector waveguides to the photodetectors is simpler than transmitting a fixed fraction of the light to the photodetectors.

FIGS. 16F-16G show views of PCB 70, in an implementation. LEDs 242 and photodetectors 335 are mounted on a surface 70a of the PCB. Temperature sensors 72 are mounted on the surface of the PCB, where each temperature sensor is located next to one of the LEDs. A baffle 1650 is located on the surface 70a.

The LEDs are arranged on a first line 70c, two photodetectors are arranged on a second line 70d, and two photodetectors are arranged on a third line 70e. The first and second lines do not intersect. The first and third lines do not intersect. The second and third lines do not intersect. The first and second lines can be parallel. The first and third lines can be parallel. The second and third lines can be parallel.

The photodetectors can be photoconductors, photodiodes (e.g., PIN photodiodes or avalanche photodiodes), phototransistors, photoresistors, photomultiplier tubes, charge-coupled devices, complementary metal-oxide-semiconductor sensors, or other types of detectors that can detect light.

Each temperature sensor can be a thermocouple, a resistance temperature detector (RTD), a thermistor, a semiconductor-based IC sensor, or another type of temperature sensor. Each temperature sensor is configured to detect the temperature of the LED that the temperature sensor is adjacent to. The measured temperature information is transferred to the processor. The processor can use the temperature information to adjust the oximetry measurement values as described in this application.

The baffle is black to minimize reflections of stray light traveling from an LEDs to a source waveguide that is not associated with the LED, from the LEDs to the photodetectors, and from a detector waveguide to a photodetector that is not associated with the detector waveguide. The baffles are also configured to block ambient light from entering the source waveguides and from being detected by the photodetectors. Thus, stray light in the lid is lowered or eliminated so that the stray light has little or no effect on oximetry measurements made by the laparoscopic oximeter. For example, direct transmission of light from the LEDs to the photodetectors that does not travel through the waveguides and patient tissue is lowered or eliminated.

Further, the baffle includes a first portion 1652 having a first height above the surface of the PCB and a second portion 1654 having a second height above the surface of the PCB. The first height is higher from the surface of the PCB than the second height. The first height being higher than the second height provides for lowering or eliminating light from traveling backwards from the forward direction of the light when emitted by the LEDs. Thus, light traveling backwards to the photodetectors can be lowered or eliminated by the height difference between the first and second heights. The first portion and the second portions are different portions, in an implementation. The first portion and the second portions are integrally formed, in an implementation.

The baffle includes apertures 1652 where the LEDs are at least partially located in the apertures. The baffle includes apertures 1654 where the LEDs are at least partially located in these apertures. With the LEDs and photodetectors located in the apertures of the baffle material, stray light traveling from the LEDs to the photodetectors in the lid is further lowered or eliminated so that the stray light has little or no effect on oximetry measurements made by the laparoscopic oximeter. That is, direct transmission of light from the LEDs to the photodetectors that does not travel through the waveguides and patient tissue is lowered or eliminated.

The openings of apertures 1652 are higher from the PCB than the openings of apertures 1653, thus light emitted from the LEDs is emitted forward of the photodetectors relative to the laparoscopic tube. With the light from the LEDs emitted forward of the photodetectors, light from the LEDs has little or no chance of traveling backward towards the photodetectors. Thus, stray reflections of light from the LEDs to the photodetectors in the lid are further lowered or eliminated so that the stray light has little or no effect on oximetry measurements made by the laparoscopic oximeter. That is, direct transmission of light from the LEDs to the photodetectors that does not travel through the waveguides and patient tissue is lowered or eliminated.

Guide blocks 1602 and 1604 are attached to the baffle and compressed onto the baffle by fasteners 1608. The back of guide block 1604 has a first portion 1606 and has second portions 1608. The first and second portions are offset in height. The first portion contacts the baffle at the portion of the baffle that includes apertures 1658, which surround the photodetectors and the second portions 1608 contact screw bosses 1660. The back of guide block 1604 has a relatively flat surface 1610 that contacts the baffle and screw bosses at the same height above the surface of the PCB. With the guide blocks pressed onto the baffle, stray reflections of light from the LEDs to the photodetectors in the lid are further lowered or eliminated so that the stray light has little or no effect on oximetry measurements made by the laparoscopic oximeter. That is, direct transmission of light from the LEDs to the photodetectors that does not travel through the waveguides and patient tissue is lowered or eliminated. And, these many devices of the lid help to improve the accuracy and quality of oximetry measurements made by the laparoscopic oximeter.

FIG. 16H shows a top view of baffle 1652 and the tops of fasteners 1608A-1608f that connect the guide blocks 1602 and 1604 to PCB 70. Fasteners 1608a-1608C can be arranged in a line. Fastener 1608b can be in a cutout 1659 of baffle 1652. The three fasteners 1608a-1608c aligned provide for uniform pressure of guide block 1602 on the baffle. Uniform pressure provides for the baffle from being bunched up, which blocks light emitted by the LEDs from entering the photodetectors.

FIG. 16I shows a view of the bottom surface 70b of PCB 70, in an implementation. The ends of screw bosses 1660 extend through PCB 70. One end of one of the screw bosses is located under electrical connector 22a. The AFE 1667 can be located in the top half of the PCB. The AFE controls the timing of control signals delivered to the LEDs to in-turn control the timing of light pulses generated by the LEDs, control the duty cycle of the light pulses, control the of intensity of the light pulse, or a combination of these light pulse characteristic. The AFE is connected to the processor of the durable unit via the electrical couplers 20a and 22a.

FIG. 16J shows a side-perspective view of PCB 70 and various elements that are connected to the PCB, in an implementation. The top 1602a of guide block 1602 and the top 1604a of guide block 1602 are equal heights “h” above a top surface 70a of PCB 70, in an implementation. One of the electrical contacts 22al of electrical connector is shown in FIG. 16I. The electrical contact can include a curved piece of metal that operates as a spring contact when contacting a corresponding electrical contact of electrical connector 20a.

The light engine includes an LED driver circuit that can be mounted on the opposite surface of the PCB from surface 70a of the PCB. The light engine can couple to the LEDs by electrical traces in the PCB. One or more of the electrical traces can be located in apertures formed in the PCB. The LED driver circuit can be connected to the processor through electrical connectors 20a and 22a to control the circuit to further control the generation of light by the LEDs.

Referring to FIGS. 16a and 16K, button 50 can be coupled to a switch 52, such as by a rocker arm 57. When button 50 is pressed by a user, the rocker arm rotates in the direction of arrow 57a about an axle 57b (e.g., about a rotation axis) and activates switch 52. Axle 57b can be connected to the PCB, to the shell 57, or to another structure of the lid. The button and switch can be configured to initiate measurement averaging features of the oximeter device.

In an implementation, the averaging mode of the laparoscopic oximeter is entered from a press and long hold of button 50. A long hold is a hold of button 50 in a pressed position for about 5 to about 10 seconds. Other button press times can be used for entry into averaging mode. The display of the laparoscopic oximeter may display an indicator that indicates the averaging mode has been entered.

After the long hold and the release of the button press of button 50, the laparoscopic oximeter enters averaging mode. The display of the laparoscopic oximeter may display an indicator that indicates the averaging mode has been entered.

As the laparoscopic oximeter generates oximetry measurement values (e.g., oxygen saturation values), the oximeter is configured to be controlled to select values for averaging. In an implementation, button 50 can be pressed for a short hold to select a displayed oximetry measurement value for selecting an initial value for an average or selecting additional values for inclusion in an average. The temporal length of a short press is less than the temporal length of a long press. For example, a short press can be less than about 5 seconds. The average of the values selected for averaging is displayed on the display of the oximeter. Further, the displays on any of the connected devices 106, 108, or 301 can receive information for the average and display the average.

The laparoscopic oximeter is configured to reset the average value and start a new average if the average button is pressed two times with a short press (e.g., less than about 1.5 seconds) between the two button presses. The laparoscopic oximeter is configured to exit the averaging mode if the averaging button is pressed for a long press (e.g., from about 5 seconds to about 10 seconds). After the press of the long hold ends, the oximeter is configured to generate oximetry measurement values (e.g., oxygen saturation values) and display the values on the display.

In an implementation, the laparoscopic oximeter is configured to determine the heart rate of a patient and display the heart rate on the display of the oximeter or other connected devices. To determine a heart rate from the measurement values generated by the photodetectors, the analog-to-digital converter samples the measurement values at a rate above 300 kilohertz. At a higher sampling rate, the pulse rate of pulsating blood can be determined from the digitized signals.

FIGS. 16L-16M show views of a fiber guard 1661, in an implementation. The fiber guard is connected to the end of housing 217 of the laparoscopic tube 15. A first portion of the fiber guard is located in the opening at the end of the housing and a second portion of the fiber guard extends outward from the housing. The outer diameter of the fiber guard can be the diameter as the outer diameter of the housing of the laparoscopic tube.

The fiber guard is connected to the portion of the housing of the laparoscopic tube that is located in the first opening 1002 of shell 35f. In an implementation, notch 1009 is formed in the fiber guard and is located in notch 1007 of the housing of the laparoscopic tube. The fiber guard can be adhered to the housing using an adhesive, such as by a heat-curable epoxy.

The fiber guard has a central opening 1661a through which the source and detector waveguides extend. Prior to installation of the source and detector waveguides, the fiber guard is installed to protect the waveguide from being damaged by potentially sharp edges of housing 217. The edges of the fiber guard at the openings of the guard can be rounded to further prevent potential damage to the waveguides when installed. The fiber guard can be plastic-type material, such as a polymer material. For example, the fiber guard can be acrylic, polycarbonate, HDPE, PVC, ABS, PTFE, polycarbonate, HIPS, acetal, resin, or other types of plastic material.

FIGS. 16N-16P show locations where an adhesive is applied to shell 35f of the lid 35a and to the housing 217 of the laparoscopic tube 15. Specifically, the adhesive can be applied at the ends of opening 1002. The adhesive is applied to hold the housing of the laparoscopic tube in opening 1002 of the lid. The adhesive can be a heat-cured epoxy. The adhesive can be applied before the fiber guard is placed into the end of the housing of the laparoscopic tube. The epoxy can be cured before the fiber guard is placed into the housing.

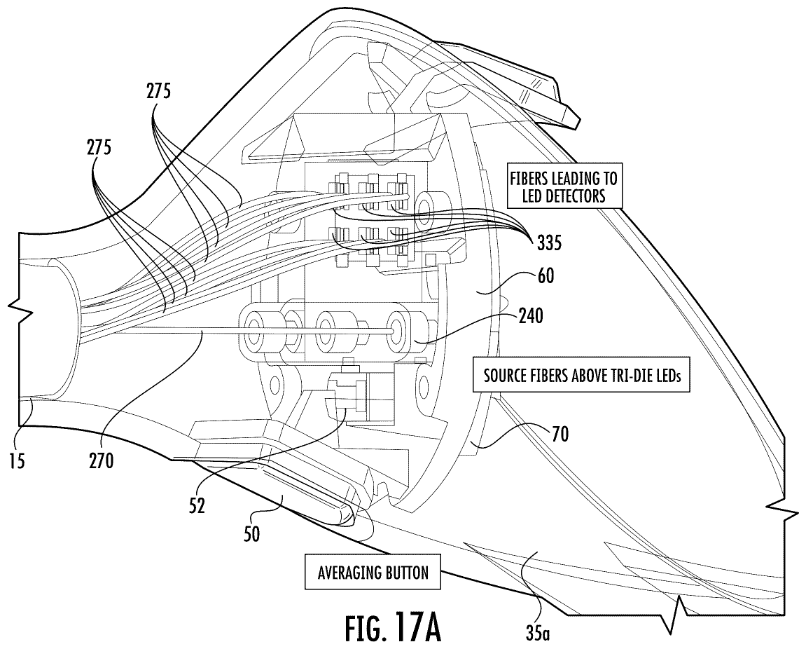

FIG. 17A shows a transparent view of the lid, in an implementation. The interior space of the lid includes light engine 240 located on a support structure 60. The light engine can be coupled to the PCB 70 through apertures formed in the support structure. The light engine can include LEDs, such as three LEDs. Each source waveguide can be optically coupled to each of the LEDs. The photodetectors can be mounted on the PCB. Ends of the detector waveguides are optically coupled to the photodetectors. The photodetectors can be LED detectors. Button 50 can be coupled to switch 52 such that when button 50 is pressed, switch 52 is pressed in turn and activated. The button and switch can be configured to initiate measurement averaging features described above.

FIG. 17B shows PCB 70 with the light engine and photodetectors mounted on the PCB. The lid includes a support structure 54 that includes apertures, slots, or both that hold the source waveguides and detector waveguides in positions to optically couple to the light engine and the photodetectors.

The durable unit contains the processor, which operates the laparoscopic oximeter 5. The processor controls the memory and the display to transmit information to the user. In an implementation, the durable unit does not contain any optical components. Control signals are sent out from the durable unit to the lid and laparoscopic tube, which takes the measurement, and digitized signals are transmitted from the lid back to the durable unit for processing. Analog signals related to the measurement can be transmitted from the lid to the durable unit, such as from the photodetectors or the amplifiers. This gives the durable unit a very long lifetime, dominated by things like ‘number of wipe-downs’ that cause labeling to come off, rather than by any physical constraint. The durable unit may not be sterilized nor particularly sterilizable by any method other than EO, but is wiped between procedures.

The lid contains the described elements pertaining to the measurement. The lid contains the LEDs, detectors, the analog front-end circuit (AFE-Texas Instruments analog front-end chip that handles the time-multiplexing and low-level electrical signal detection in the measurement), and all the transmission pathways for the photons. The AFE may be coupled between the photodetectors and the ADC in the durable unit.

A method of operation of the laparoscopic oximeter includes:

1. Signals from each combination of LED and photodetector are digitized.

2. These ‘diffuse reflectance’ curves of voltage vs distance are fitted to a database of curves corresponding to different optical properties.

3. The ‘best matching’ curve is used to determine the optical properties (absorption coefficient pa and the scattering coefficient μs, such as the reduced scattering coefficient μs′, of the sample at that wavelength.

4. μa as a function of wavelength is processed via a spectral decomposition process to estimate chromophore (hemoglobin) concentrations.

5. Hemoglobin concentrations convert into oxygen saturation values, StO2. At the end of step 5, StO2 values can be scaled to take up more of the physiological range.

In one implementation, the durable unit does not enforce a fixed temperature setpoint for the generation of oximetry measurement values. Rather, the durable unit corrects oximetry measurement values for thermal variability. Information for thermal variability is generated by one or more thermistors that are located on PCB 70 near the LEDs. The use of thermistors for generating thermal variability information for correcting oximetry measurement values includes faster startup times for use of the laparoscopic oximeter rather than waiting for the oximeter to thermally stabilize and allow for lower power consumption because the oximeter does not have to be powered on for a relatively long time waiting for thermal stabilization and power does not need to be expended actively heating the oximeter, such as by operating the LEDs to generate heat.

In an implementation, shifts in the internal temperature of the measurement unit result in changes in the raw signals for oximetry measurements that are generated by the measurement unit, and therefore can effect changes in measured oxygen saturation values or other oximetry measurement values generated by the laparoscopic oximeter. Two different thermal corrections can be applied to the oximetry measurement values to correct the oximetry measurement values when the internal temperature of the measurement unit changes during use of the laparoscopic oximeter.

First, the durable unit corrects for changes in the total number of photons that get through the optical path of the measurement unit. Correcting for the total number of photons that get through this optical path takes into account geometric changes in the measurement unit due to thermal expansion or thermal contraction of the system components, such as changes in the optomechanical coupling of the LEDs to the source waveguides and changes in the optomechanical coupling of the photodetectors to the detector waveguides. Correcting for the total number of photons that get through this optical path takes into account changes in LED output efficiency and takes into account changes in detector efficiency (i.e., sensitivity). Second, the durable unit corrects for the spectral shift in the LEDs as a function of temperature.

In an implementation, during the manufacture of a measurement unit, changes in the total number of photons that get through the optical path of the measurement unit versus internal temperature of the measurement unit (e.g., temperature changes in the lid of the measurement unit) are determined for a number of different temperatures. Changes in the internal temperature of the measurement unit can be monitored by the thermistors located adjacent to the LEDs on the PCB located in the lid as the total number of photons that are detected by the photodetectors varies. The temperature of the laparoscopic oximeter can be changed by changing the ambient temperature (local atmosphere in which the oximeter is located) of the location in which the oximeter is located. Thermal correction coefficients for the total number of photons that get through the optical path versus temperature are stored in the memory of the measurement unit. The thermal correction coefficients are used by the laparoscopic oximeter to adjust the raw data generated for oximetry measurement made by the laparoscopic oximeter based on temperature measurements made by the thermistors when the oximeter generates the raw data. The raw data adjustments can be adjustments for the total number of photons that are detected by the photodetectors for a signal generated by one or more of the thermistors of the laparoscopic oximeter. Adjustments of the raw data for the oximetry measurements can be adjusted by mathematically applying the thermal correction coefficient to the raw data, such as by multiplication, division, addition, subtraction, or other mathematical function. Temperature measurements can be relative temperature measurements or absolute temperature measurements.

In an implementation, the thermal correction coefficients are determined by measuring the reflectance from an integrating sphere based on light emitted by a laparoscopic oximeter over defined ambient temperatures while recording temperature from the thermistors. Use of an integrating sphere allows for the laparoscopic oximeter to generate optical signals that vary over temperature and are independent of illumination angle, detection angle, and contact variability. That is, the measured signal contains all thermal effects of the illumination and detection hardware. The thermal correction coefficients can be used is a second-order polynomial Cf, where Cf is a correction factor for correcting oximetry measurements (such as a raw signal). Cf includes variable for the temperature (determined by one or more of the thermistors), and A, B, and C that are the correction coefficients in the second order polynomial. The function is fit to the signal versus temperature data, where the signal is normalized to 25 degrees Celsius to determine the thermal correction coefficients. Subsequent thermal corrections to the raw data are implemented by calculating the correction factor from a measured temperature and known thermal correction coefficients, and dividing the raw signal by the factor Cf. Each of the wavelengths generated by the laparoscopic oximeter are associated with their own set of thermal correction coefficients. For example, if the laparoscopic oximeter generates and emits six wavelengths (e.g., three wavelengths for each LED) a total of 18 thermal correction coefficients are stored on the memory in the lid of the measurement unit. More or fewer thermal correction coefficients can be stored in the memory if the laparoscopic oximeter is configured to emit more or fewer wavelengths.

In an implementation, during the manufacture of a measurement unit, changes in the spectrum (e.g., wavelength changes) of light emitted by the LEDs versus temperature are determined for a number of different temperatures. Wavelength changes can include wavelength shifts, wavelength spreading (spectrum spreading), or both. Wavelength shift coefficients (e.g., correction coefficients) for the changes in the spectrum (e.g., wavelengths) of light emitted by the LEDs versus temperature are stored in the memory of the measurement unit.

Specifically, as the temperatures of the LEDs change, the wavelengths of light generated by the LEDs shifts and the molar absorptivity constant, ¿, of patient tissue shifts with the changed wavelengths. The determined absorption coefficient pa shifts with the shifted molar absorptivity constant, which alters the oximetry measurement values generated by the laparoscopic oximeter, such as the oxygen saturation values.

In an implementation, the determined absorption coefficients are used to calculate oxygen saturation values by implementing spectral decomposition using the net analytic signal (NAS) vectors. NAS vectors are a mathematical concept that represent a portion of a measured spectrum that is unique and specific to specific chromophores (e.g., oxygenated hemoglobin, deoxygenated hemoglobin, or both) when other chromophores are present in patient tissue substances.

The NAS vectors are linearly transformed versions of the oxygenated hemoglobin and deoxygenated hemoglobin molar extinction values at each wavelength. To produce stable oxygen saturation values for varying temperature, the NAS vectors are altered based on wavelength shift coefficients stored in the memory of the measurement unit versus temperature of the interior of the measurement unit, such as the interior of the lid. Adjusting the NAS vectors for varying temperature is sometimes referred to as wavelength correction.

Implementing the wavelength correction includes characterizing the LED wavelength shifts versus temperature change. To do this, spectra of the LEDs are individually measured as ambient temperature is varied for a number of oximeters 5 to provide a linear trend of average wavelength versus temperature for each LED. The slopes of this trend can be used to inform the calibration of other oximeters.

To calibrate individual measurement units, a single spectra is measured for each LED of a measurement unit versus internal temperature of the measurement unit (e.g., of the lid). The average wavelength of the spectra is then calculated. The previously determined slopes of average wavelength versus temperature are used to normalize the LED average wavelengths to 25 degrees Celsius. Since the wavelength versus temperature correlation is known, the NAS vector elements can be calculated over temperature for each measurement unit. A second-order fit is applied to each NAS vector element versus temperature to determine the three wavelength shift coefficients. The three-wavelength shift coefficients are determined from a second order polynomial NASi where the second order polynomial includes the temperature T, and Ai, Bi, and Ci are the wavelength shift coefficients. In one implementation, each NAS vector contains 3 elements corresponding to 3 wavelengths of light emitted by each LED, and for 2 LED sources and 2 NAS vectors (oxygenated hemoglobin (HbO2) and deoxygenated hemoglobin (Hb)), there are a total of 3 (A, B, and C wavelength shift coefficients)*3 (wavelengths)*2 (sources)*2 (HbO2 and Hb vectors)=36 wavelength shift coefficients. These wavelength shift coefficients are stored in the memory of a measurement unit. And, for every measurement, a unique set of NAS vectors are calculated based on the temperature reading of the thermistor. The NAS vector can contain other numbers of elements if the LEDs emit more or less than 3 wavelengths of light. As is well understood by those of ordinary skill in the art, after the NAS vectors are adjusted for temperature variation of the interior of the lid during an oximetry measurement made by a laparoscopic oximeter, relatively simple vector multiplication and simple arithmetic are used by the oximeter to determine estimates for oxygenated hemoglobin concentration and deoxygenated hemoglobin concentration, and thereafter oxygen saturation.

Second, the durable unit samples tissue that is wet, as the laparoscopic oximeter is used inside the body of a patient. A scaling equation between steps 3 and 4 (described above) converts μa as determined from a fit of oximetry measurement values to simulated reflectance curves of dry tissue to values for ua that would have been determined if the oximeter had fit oximetry measurement values to simulated reflectance curves for wet tissue.

In an implementation, the software used by the laparoscopic oximeter is embedded software. This can be enabled by the AFE, which can be designed to run measurement sequences at high speed. Without the need for signal modulation or measurement at ultra-high speeds, where the embedded software may not be used.

In an implementation, a first calibration is performed for the lid and measurement unit. The durable unit handles already-digitized measurement information. Each lid and laparoscopic tube is calibrated with two types of key measurements. First, there is a measurement against a sample (e.g., a tissue phantom) with known optical properties. The amount of signal that should be generated using the tissue phantom is known for each LED-photodetector pair, and can save calibration coefficients to scale up or down as needed to normalize to our lookup database.

A second calibration is a wavelength measurement calibration. Individual LEDs have some variability in the shape or exact wavelength of their spectra, and knowing this provides an accurate spectral decomposition in the algorithm when predicting hemoglobin levels. The spectrum is parameterized and saved to the memory (e.g., EEPROM) of the lid 35a.

In an implementation, for a number of laparoscopic oximeters, measurements are taken to appropriately parameterize the oximeter's responses to the presence of liquid on a sample and the reaction to temperature.

Measurements are taken on a series of tissue phantoms with known optical properties (including a calibration phantom) to understand the optical property predictions made when wet and dry. The measurements provide for a conversion of the oximetry measurement values for the wet to dry conversion of the oximetry measurement values, such as μa.

In an implementation, a number of laparoscopic oximeters are used with a spectrometer to measure the wavelengths of transmitted light generated by the LEDs or at a tissue phantom to measure device response, including photodetector response, to understand the changes in expected spectra and signal magnitudes, respectively, as a function of temperature. Device responses can be used for determining the calibration coefficient stored in the oximeter and used for calibrating oximetry measurements.

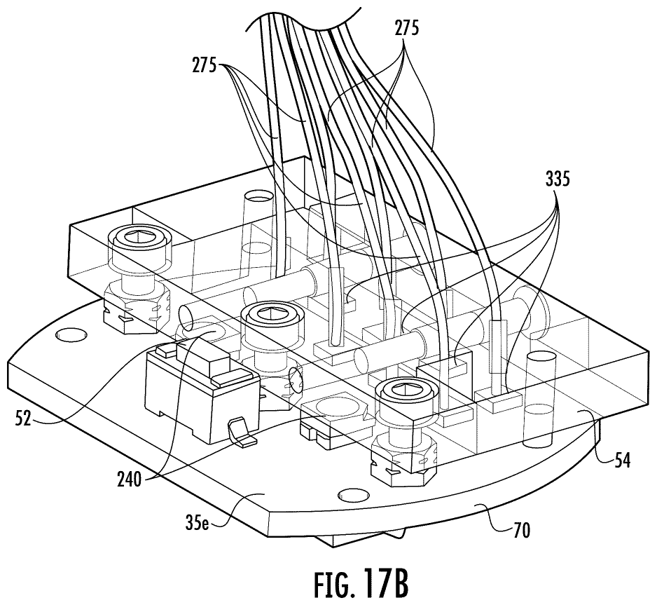

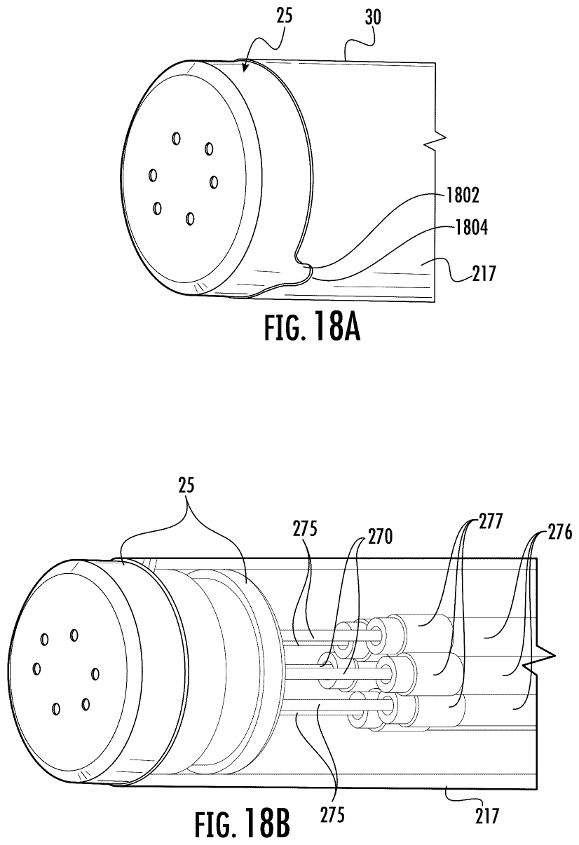

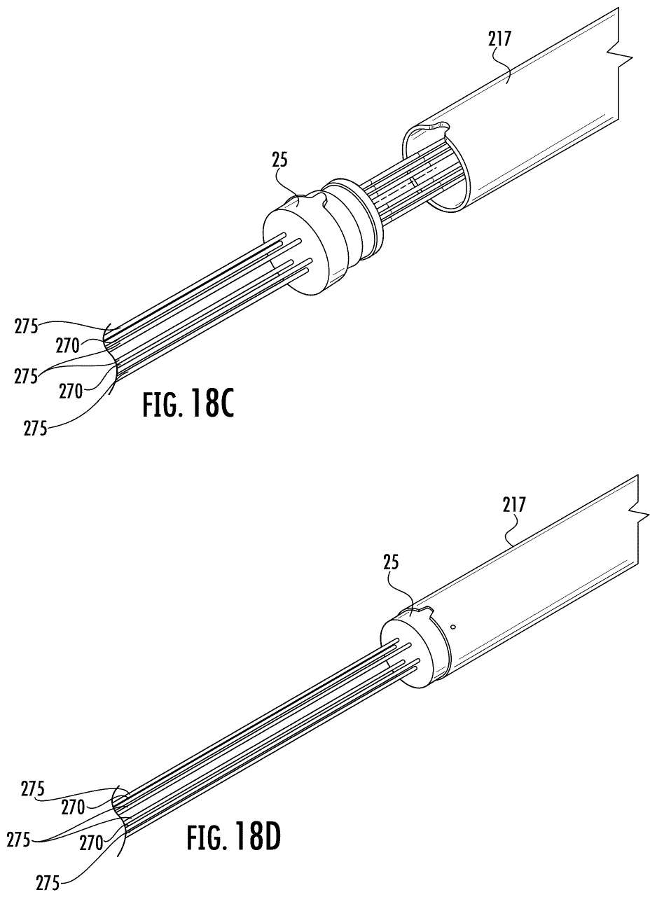

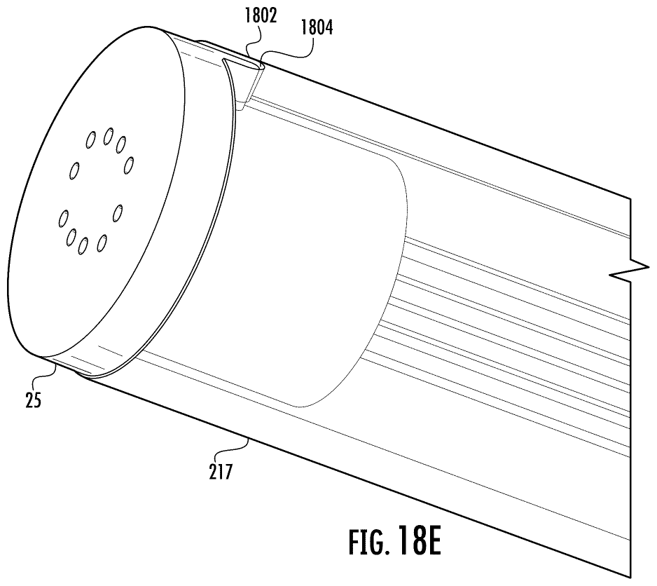

FIGS. 18A-18B show end portion 30 of the housing 217 of the laparoscopic tube 15 and the sensor head 25, in an implementation. The sensor head includes a tab 1802 that is positioned in a notch 1804 of housing 217 when the sensor head is located in housing. The Tab and notch are a key system that orients the sensor head in a known orientation with respect to the housing. In an implementation, notch 1804 and notch 1007 (see FIG. 10D) are aligned on the housing.

FIG. 18B shows a transparent view of the housing, in an implementation where the laparoscopic oximeter includes 2 source waveguides and 4 detector waveguides. FIG. 18E shows a transparent view of the housing, in an implementation where the laparoscopic oximeter includes 2 source waveguides and 8 detector waveguides. Each source waveguide 270 and detector waveguide is located in a corresponding tube 276. Each tube 276 is a plastic or rubber type of material that can protect the waveguides in the tube from becoming damaged. In a specific implementation, each tube is black silicon and has an inner diameter of about 1/32 of an inch and an outer diameter of about 1/16 of an inch. In one implementation, the tubes are inserted into the apertures formed in guides 1602 and 1604 and are adhered in the apertures with an adhesive. After the adhesive is cured, the tube and waveguides are trimmed and the waveguides are polished. In an implementation, collars 277 are placed at the ends of tubes 276. The collars can be affixed to the tube by an adhesive, shrink wrapping, or another connection type. The collars can have a variety of colors that identify the specific waveguides for ease of assembly of the sensor head.

The top surface of the laparoscopic tube at the sensor head is polished flat, as shown in FIGS. 18A-18B and 18E. The components on the surface of the sensor head can be substantially coplanar. The top surface of the sensor head is substantially perpendicular to the laparoscopic tube, but can be oriented at other angles (such as 45 degrees) in some implementations. Variant angles may use variant algorithms for determining oximetry information, such as oxygen saturation.

FIGS. 18C-18D show the source and detector waveguides being inserted into the housing 217 of the laparoscopic tube for assembly. The waveguides are inserted through the apertures formed in the sensor head. Thereafter, the waveguides are inserted into housing 217 of the laparoscopic tube. The sensor head can be epoxied into the opening at the end of the housing. Thereafter, the ends of the waveguides and the face of the sensor head are polished flush.

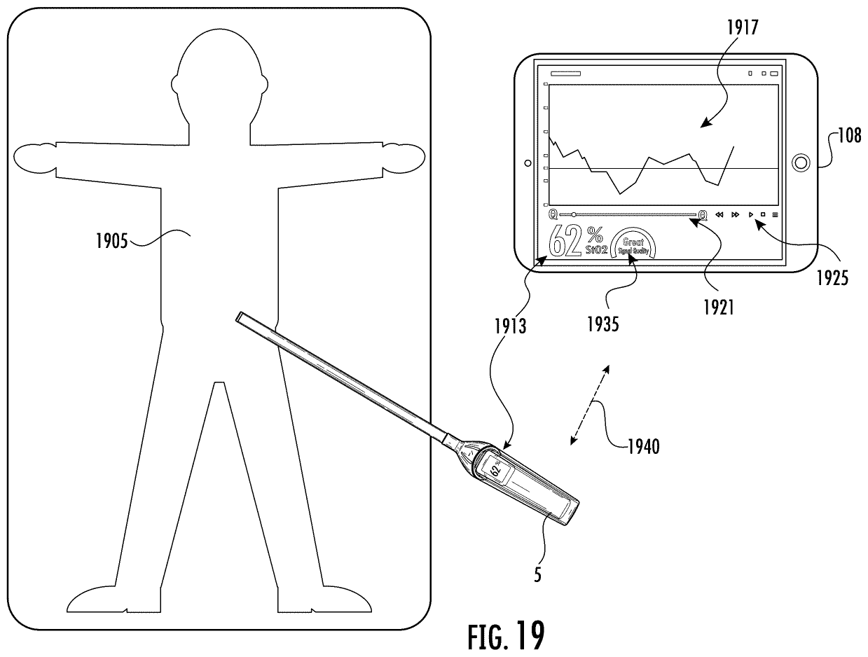

FIG. 19 shows an example use of laparoscopic oximeter 5 in an operating room with a patient 1905 during an operation. When the laparoscopic oximeter measures oxygen saturation for patient tissue, the oximeter can display oxygen saturation values 1913 on the display of the oximeter. The oximeter can display a numerical value of the percentage of oxygen saturation, a bar level indication of the oxygen saturation value, a graph of oxygen saturation values over time, or any combination of these indicators. The oximeter can display a battery level indicator that indicates an amount of charge remaining in the battery. The battery level indicator can be a bar level indicator with a number of bars displayed in a first color to indicate a charge state. For example, the bar level indicator may include five bars. If the charge state of the batteries is 80 percent, then four of the five bars may be displayed as green to indicate an 80 percent charge state and the fifth bar may be displayed as another color, such as black to indicate a 20 percent discharge state of the batteries.

The displayed oxygen saturation values can be generated by the durable unit in real time based on oximetry measurement information generated by the measurement unit and provided to the durable unit. Oxygen saturation values calculated by the durable unit in real time and can, in turn, be displayed in real time on the display of the laparoscopic oximeter. The laparoscopic oximeter can generate and display essentially an unlimited number of oxygen saturation values during a procedure, limited by the life of the batteries.

The oximeter can also transmit (e.g., via a Bluetooth transmission 1940) the oxygen saturation values 1913 to a computing device 108 (e.g., a tablet computer) located in the operating room with the patient and oximeter. Communications between the laparoscopic oximeter and the computing device can be encrypted. The laparoscopic oximeter can decrypt the encrypted communications transmitted from the computing device to the laparoscopic oximeter. And, the computing device can decrypt the encrypted communications transmitted from the laparoscopic oximeter to the computing device. Thereafter, the computing device can display the decrypted oxygen saturation value 1913, display a graph 1917 of oxygen saturation values over time 1917, or both. The computing device can display a graphic 1935 that indicates the quality of contact between patient tissue and the sensor head of the oximeter. The computing device can display a control bar that allows for enlarging the displayed graph 1917 or shrinking the displayed graph on the display. The computing device can display a control 1925 for pausing the time for the displayed graph 1917 or allowing the graph to proceed to display oxygen saturation values as time advances. The computing device can display a bar level indication of the oxygen saturation value. The computing can display a battery level indicator that indicates an amount of charge remaining in the battery.

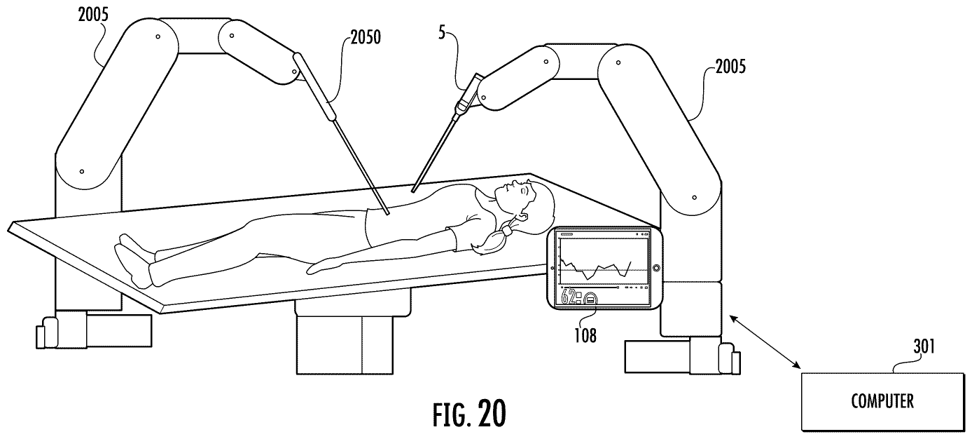

FIG. 20 shows an example use of laparoscopic oximeter 5 in an operating room with a patient, in an implementation. The oximeter is connected to a robotic arm 2005, where the robotic arm is configured for robotic manipulation of the oximeter for use with the patient. The robotic arm is connected to a computer system 301 that is configured to control the movement of the robotic arm. The computer system 310 can be connected to the robotic arm via a wired connection or a wireless connection. The computer system 310 can be connected to the robotic arm via a direct wireless connection (e.g., a Bluetooth connection) or a Wi-Fi connection. The computer system can be connected to a server system 106 via the Wi-Fi network. Communications between the computer system and the robotic arm can be encrypted communications, where the robotic arm includes a durable unit that can decrypt the encrypted communications to control the arm using the decrypted communications. Computer system 310 can be in the operating room in which the robotic arm is located, in another room of the hospital in which the robotic arm is located, or at another location. The laparoscopic oximeter can be connected to a computing device 108, such as a tablet computer, described above with respect to FIG. 19.

The robotic arm can be configured to move along the Cartesian coordinates, through the angles of the polar coordinates, through the angles of the spherical coordinates, through a combination of the Cartesian and polar coordinates, or through a combination of the Cartesian and spherical coordinates.

The laparoscopic oximeter can be used with other laparoscopic tools 2050 that are controlled by another robotic arm. In an implementation, a gel is applied to the face of the sensor head to improve physical and optical contact of the face of the sensor head with the interior tissue of the patient 1905. The gel can be a water-based gel.

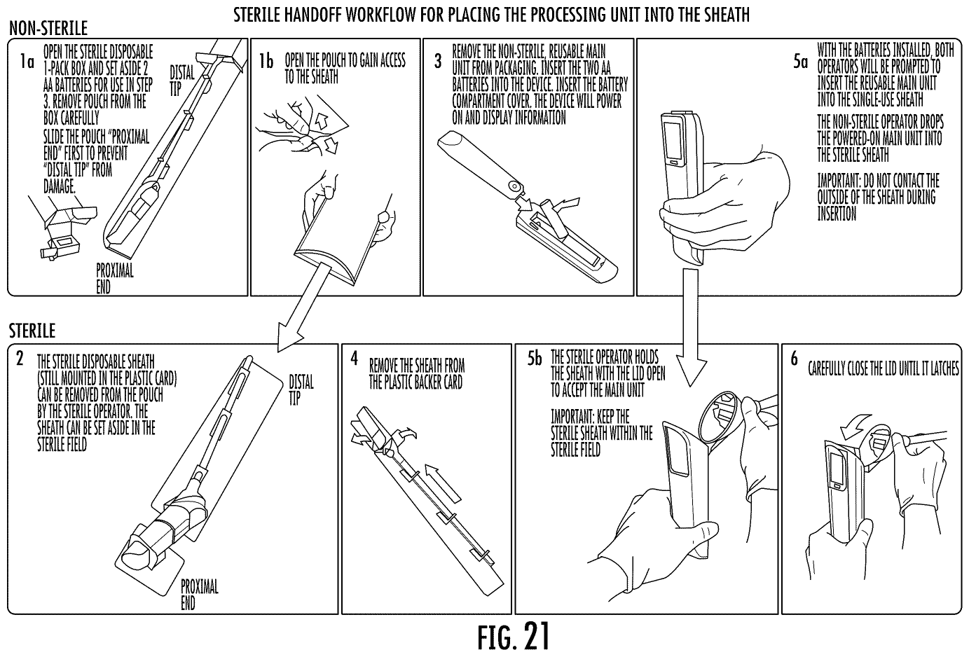

FIG. 21 shows a process for placing the durable unit into the measurement unit for a surgery, in an implementation. At a step 1a, a non-sterile nurse or other medical personal that is located in a non-sterile field of an operating room opens a sealed package in which a measurement unit 12 is located. For example, the non-sterile nurse opens the sterile disposable 1-pack box and set aside 2-AA batteries for use in the step 3. The non-sterile nurse removes pouch from the box carefully. The non-sterile nurse slides the pouch “proximal end” first to prevent “distal tip” from damage. The proximal end is identified in FIG. 21.

At step 1b, the non-sterile nurse tilts the package allowing the measurement unit to slide from the package into the sterile field of the operating room. At step 1b, the non-sterile nurse does not touch the measurement unit. If the non-sterile nurse inadvertently touches the measurement unit, the process is restarted with a new durable unit and measurement unit. For example, the non-sterile nurse opens the pouch to gain access to the sheath.

At step 2, a sterile nurse or other medical personal working in the sterile field of the operating room can help remove the measurement unit from the package without touching the package. For example, the measurement unit can be slid onto a sterile medical tray or sterile table. For example, the sterile disposable sheath (still mounted in the plastic card) can be removed by the sterile nurse. The sheath can be set aside in the sterile field.

At step 3, the non-sterile nurse removes the durable unit from a package in which the durable unit is located. The non-sterile operator can thereafter remove the battery housing from the durable unit, place batteries in the durable unit, and replace the battery housing on the durable unit. For example, the non-sterile nurse removed the non-sterile, reusable main unit (e.g., the durable unit) from the packaging. The non-sterile nurse inserts the two AA batteries into the device (e.g., the durable unit). The device (e.g., the durable unit) will power on and display information.

At step 4, a sterile nurse or other sterile medical personal removes the measurement unit from any additional packaging material that the measurement unit is in. The lid 35a of housing 35 can opened. For example, the sterile nurse removes the sheath (e.g., the measurement unit) from the plastic backer card.

At step 5a, the non-sterile operator can position the durable unit for insertion into the sheath 35b without touching any portion of the measurement unit. For example, with the batteries installed, both the operators (e.g., the sterile and non-sterile nurses) will be prompted to insert the reusable main unit (e.g., the durable unit) into the singe-use sheath. The non-sterile nurse drops the powered-on main unit (e.g., durable unit) into the sterile sheath. It is important for the non-sterile nurse not to contact the outside of the sheath during insertion.

At step 5b, the sterile nurse can hold the lid of the housing open while the non-sterile nurse places the durable unit into the sheath. The non-sterile nurse should not touch any portion of the measurement unit and the sterile nurse should not touch any portion of the durable unit. If the non-sterile nurse touches any portion of the measurement unit, the process of placing a durable unit into a measurement unit begins again with new measurement. If the sterile nurse touches any portion of the durable unit, the sterile nurse may have to resterilize. For example, The sterile nurse holds the sheath with the lid open to accept the main unit (e.g., the durable unit). It is important to keep the sterile sheath within the sterile filed.

At step 6, the sterile nurse closes the lid onto the sheath sealing the durable unit in the housing forming a fully function laparoscopic oximeter. For example, The sterile nurse carefully closes the lid until it latches.

Closing of the lid onto the sheath provides contact to be made between electrical contacts of the lid and electrical contacts of the durable unit. The gold pads on the durable unit to allow for wiping down the durable for cleaning, sterilizing, or disinfecting. If electrical contact is ever lost between the electrical contact of the lid and the electrical contact of the durable unit, the durable unit knows that it is not properly connected and can provide a warning on the display that electrical contact has been lost. Thereafter, an assessment can be made of whether the lid has become open. If the lid has become open during the operation, a new measurement unit can be attached to the durable unit.

Lid 35a can include a memory (e.g., a flash memory EEPROM) that stores device-specific calibration information for a measurement unit. The memory is located in the NFC device of the lid, in an implementation. In an alternative implementation, the memory is located on PCB 70. When electrical contact is made between electrical connectors 20a and 22a, the calibration information can be transferred from the measurement unit to the durable unit for use by the durable unit when oximetry measurements are made by the laparoscopic oximeter.

Placing the durable unit in an incorrect orientation in the sheath of the housing cannot occur due to the exterior shape of the durable unit and the interior shape of the sheath, in an implementation. That is, the durable unit cannot be placed sufficiently inside the sheath so that the lid can be rotated to a closed orientation with the sheath if the durable unit is rotated by 90 degrees about the longitudinal axis, for example, from a “correct” orientation or 180 degrees about the longitudinal axis, for example, from the correct orientation. The correct orientation of the durable unit in the sheath is an orientation where display 224 of the durable unit is under window 35g of the sheath as shown in FIG. 10C. Because the durable unit cannot be placed inside the sheath in an incorrect orientation with the lid closed, the electrical contacts 20a and 22a will not be damaged by the lid being rotated towards the closed orientation.

In an implementation where the durable unit cannot be placed fully inside the sheath in an incorrect orientation, the curvatures of the top surface 10a and bottom surface 10b of the durable unit and the inside-top surface 35s and inside-bottom surface 35r of the sheath are shaped to prevent insertion in an incorrect orientation.

For example, the top surface 10a of the durable unit and the inside-bottom surface 35r (FIGS. 10C and 10E) of the sheath have curved shapes with different radiuses of curvature that prevent inserting the durable unit inside the sheath in an incorrect orientation. The curve of the top surface 10a curves from a left side 10x of the durable unit to a right side 10y of the durable unit. The curve of the inside-bottom surface 35r of the sheath curves from a left side 35x of the durable unit to a right side 35y of the durable unit. The curve of the top surface 10a of the processing has a first radius of curvature. The curve of the inside-bottom surface 35r of the sheath has a second radius of curvature. And, the first radius of curvature is greater than the second radius of curvature, in an implementation.

The display on the top surface of the durable unit can be similarly curved from the left to right side of the durable unit and can have a third radius of curvature. The third radius of curvature is greater than the second radius of curvature, in an implementation.

Further, the bottom surface 10b of the durable unit and the inside-top surface 35s of the sheath have curved shapes with different radiuses of curvature that prevent inserting the durable unit inside the sheath in an incorrect orientation. The curve of the bottom surface 10b curves from a left side 10x of the durable unit to a right side 10y of the durable unit. The curve of the inside-top surface 35s of the sheath curves from a left side 35x of the durable unit to a right side 35y of the durable unit. The curve of the bottom surface 10b of the processing has a fourth radius of curvature. The curve of the inside-top surface of the sheath has a fifth radius of curvature. And, the fourth radius of curvature is less than the fifth radius of curvature, in an implementation.

The bottom of the durable unit and the interior bottom of the sheath can both be angled at non-zero angles that are approximately the same angles and that slope in the same direction when the durable unit is placed into the sheath in the correct orientation (display and widow facing the same direction). If the durable unit is placed in the sheath in any other orientation than the correct orientation, the bottom surface of the durable unit and the inside bottom surface of the sheath will not slope in the same direction and the durable unit will not slide far enough into the sheath so that the lid can be closed onto the sheath.

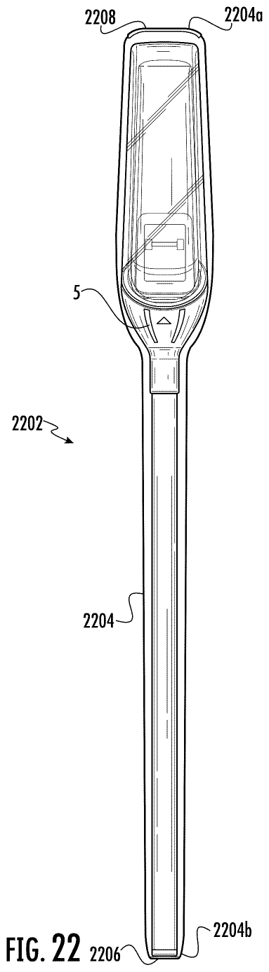

FIG. 22 shows the laparoscopic oximeter 5 located in a sheath 2202, in an implementation. The sheath is configured to inhibit contaminants from contacting the oximeter during use. The material of the sheath can have a pore size that inhibits one or more of dirt, fungus, bacteria, viruses, and prions from passing through the sheath.

The sheath includes a first portion 2204 and a second portion 2206. The first portion includes a boundary layer formed of a first material. The boundary layer has a first flexibility. The second portion includes a transparent window formed of a second material. The boundary layer has a first rigidity and the second portion has a second rigidity. The rigidity of the first portion is less than the rigidity of the second portion. The first portion can include a first opening 2204a at a first end and a second opening 2204b at a second end. The first and second ends of the bag are distally located with respect to each other. The first portion is continuous between the first and second openings.

The transparent window of the second portion 2206 can be disk-shaped. The transparent window is adhered to the first portion 2204 of the sheath at the second opening 2204b. The transparent window can be epoxied to the first portion, plastic-welded to the first portion, or otherwise attached to the first portion. The transparent window seals the second opening closed. The transparent window can be a relatively rigid material, such as rigid plastic or glass. The transparent window can have a thickness from about 0.1 millimeters to about 1 millimeter. The transparent window that is configured to allow the light transmitted from the laparoscopic oximeter to pass through the window into patient tissue. The transparent window is also configured to allow light reflected or scattered from patient tissue to pass through the window back to the sensor head.

The transparent window can have opposite surfaces that are substantially parallel. The first surface forms a portion of the inner surface of the sheath. The second surface forms a portion of the outer surface of the sheath. The first surface of the transparent window is configured to be in flush contact with the probe face of the sensor head when the laparoscopic oximeter is in the sheath. In an implementation, the transparent window has an index of refraction that is approximately the same as the index of refraction of the source waveguides and of the detector waveguides.

The first opening 2204a of the sheath is configured to allow the laparoscopic oximeter to be inserted into the sheath for use. The first opening 2204a is configured to be closed and sealed when the laparoscopic oximeter is located in the sheath for use. A seal material 2208 can be adhered to a surface of the first portion adjacent to the first opening. The seal material allows for a first portion of the first material at the first opening to be adhered to a second portion of the first material at the first opening to seal the opening. The first and second portions of the first material at the first opening are different portions of the first material, in an implementation. The seal material can be covered with a tear-away cover, where the tear-away cover can be removed from the seal material when the first opening is to be sealed by the seal material when the laparoscopic oximeter is in the sheath.

As described briefly above, the first portion of the sheath is formed of a flexible material, a rubberized material, a plastic or a plastic-type material, where the material blocks patient tissue and fluid and other debris from contacting the portion of the laparoscopic oximeter that is covered by the sheath. The sheath can also block one or more prions, viruses, bacteria, fungi, and other contaminants from contacting the portion of the laparoscopic oximeter that is covered by the sheath.

The first portion of the sheath can be formed of polycarbonate, latex rubber, polyurethane, polyisoprene, nitrile, silicone, polymer, plastic, cellophane, polyester film (i.e., BoPET film, sold under the trademarked brand names Mylar, Melinex, Hostaphan, and other trademarked names), polyethylene film (e.g., low density polyethylene (LDPE), linear low density polyethylene (LLDPE), medium density polyethylene (MDPE), high density polyethylene (HDPE, also referred to as HD), or a combination of one or more of these materials that form a film), a combination of ethylene methyl acrylate copolymer and polyethylene film, nylon film, polyvinyl chloride film with or without a plasticizer, or other materials that prevents tissue, fluid, prions, viruses, bacteria, fungi or other contaminants from contacting the laparoscopic oximeter. The first portion of the sheath can be a laminate of any one or more of the film materials mentioned above, such as a laminate of BoPet and polyethylene (e.g., LLDPE), another polyester layer, or another material. The sheath can be a film that is a mixture of materials that are used for making film, such as a mixture of LLDPE and HDPE.

The second portion of the sheath can be formed of a plastic-type material or glass. The plastic-type material can be a relatively rigid plastic-type material, such as a type of rigid polymer. For example, the material can be acrylic, polycarbonate, HDPE, PVC, ABS, PTFE, HIPS, acetal, resin, or other types of plastic material.

The sheath allows for the measurement unit to be used multiple times. The sheath can be sterilized after a first use with a first patient for a second use with a second patient. The sheath can be sterilized as described above via wipe down with a sterilizing agent, by EtO gas, or other method and sterilant.

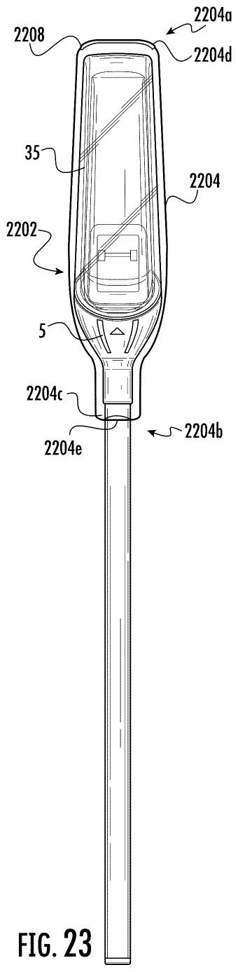

FIG. 23 shows the laparoscopic oximeter 5 located in a sheath 2202, in an implementation. The sheath is configured to inhibit contaminants from contacting housing 35 during use. The material of the sheath can have a pore size that inhibits one or more of dirt, fungus, bacteria, viruses, and prions from passing through the sheath.

The sheath includes a boundary layer 2204 that is a continuous layer from a first end 2204a of the layer to a second end 2204b of the layer. The sheath includes a first opening 2204d at a first end 2204a and a second opening 2204c at a second end 2204b. The first and second ends of the sheath are located at distal ends of the sheath. The first opening is a larger opening then the second opening. In an implementation, the diameter of the first opening is larger than the diameter of the second opening, such as 2 times to 5 times larger. The first opening is sufficiently large so that the laparoscopic oximeter can be slid into the sheath via the first opening. In an implementation, the second opening is sufficiently small so that the laparoscopic tube can slide into the second opening, but the second opening is not large enough so that housing 35 cannot slide into the second opening. In another implementation, the second opening is sufficiently small so that the laparoscopic tube can slide into the second opening and the tip of housing 35 can slide into the second opening, but the body of the housing, where the housing begins to widen cannot slide into the second opening. The housing starts to widen from about 0.5 centimeters to about 3 centimeters from the end of the housing.

The sheath includes a retaining ring 2204e that is located at the second end 2204b of the sheath. The retaining ring 2204a is connected to the boundary layer and is adjacent to the second opening 2204c. The retaining ring can be adhered to the outside surface or the inside surface of the boundary layer. The retaining ring is formed of an elastic material so that the ring can stretch and retract. The retaining ring has a higher elasticity than the boundary layer, in an implementation. When an end portion of the laparoscopic tube or the tip of housing 35 is located in the second opening, the retaining ring compresses the boundary layer onto the tube or housing portion. The retaining ring holds the boundary layer against the tube or tip of the housing to inhibit contaminants from contacting the upper portion of the tube or tip of the housing.

The sheath allows for the measurement unit to be used multiple times. The sheath can be sterilized after a first use with a first patient for a second use with a second patient. The sheath can be sterilized as described above via wipe down with a sterilizing agent, by EtO gas, or other method and sterilant.

This detailed description describes examples of implementations with specific measurements, angles, values, dimensions, shapes, and orientations. These example implementations are not intended to be exhaustive or to limit the described implementations to the precise form described.

The measurements, for example, in millimeters or centimeters are approximate values. The values can vary due to, for example, measurement or manufacturing tolerances (as will be understood by those of ordinary skill in the art) or other factors (as will be further understood by those of ordinary skill in the art). A measurement can vary, for example, by plus or minus 1 percent, plus or minus 5 percent, plus or minus 10 percent, plus or minus 15 percent, plus or minus 20 percent, plus or minus 1 to 5 percent, plus or minus 5 to 10 percent, or plus or minus 15 to 20 percent. Further, the measurements are for a specific implementation of the device, and other implementations can have different values, such as certain measurements, dimensions, or both. The measurements can be made longer to accommodate smaller hands or larger hands or to access tissue in a particular location of a patient's body.

For the specific implementations described, some specific values, ranges of values, and numbers are provided. These values indicate, for example, dimension, angles, ranges, frequencies, wavelengths, numbers, a relationship (e.g., relative value), and other quantities (e.g., numbers of sensors, sources, detectors, diodes, fiber optic cables, and so forth). Some measurements are for a specific implementation of the device, and other implementations can have different values, such as certain dimensions that are made larger for a larger-sized product or made smaller for a smaller-sized product. The device may be made proportionally larger or smaller by adjusting relative measurements proportionally (e.g., maintaining the same or about the same ratio between different measurements). In various implementations, the values (or numbers or quantities) can be the same as the value given, about the same as the value given, at least or greater than the value given, or can be at most or less than the value given, or any combination of these. The values (or numbers or quantities) can also be within a range of any two values given or a range including the two values given. When a range is given, the range can also include any number within that range to any other number within that range.

The dimensions, for example, along an axis, a rotational orientation, or both are approximate values. The dimensions can be in values, directions, angles, or any combination of these dimensions. Dimensions, for example, of values in millimeters or centimeters, of directions along an axis or at an angular orientation relative to an axis, or of an angular orientation are approximate values. The values, direction, and angles can vary due to, for example, measurement or manufacturing tolerances or other factors. A dimension can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, plus or minus 20 percent, plus or minus 0.1 to 0.2 percent, plus or minus 0.2 to 0.5 percent, plus or minus 0.5 to 1 percent, plus or minus 1 to 5 percent, plus or minus 5 to 10 percent, plus or minus 10 to 15 percent, or plus or minus 15 to 20 percent.

The shapes, for example, a geometric shape can be approximate shapes. The shapes can be in values, directions, angles, terms, or any combination of these shapes. The shapes can vary due to, for example, measurement or manufacturing tolerances or other factors. A shape can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, plus or minus 20 percent, plus or minus 0.1 to 0.2 percent, plus or minus 0.2 to 0.5 percent, plus or minus 0.5 to 1 percent, plus or minus 1 to 5 percent, plus or minus 5 to 10 percent, plus or minus 10 to 15 percent, or plus or minus 15 to 20 percent.

The orientations, for example, parallel, perpendicular, transverse, and angle are approximate values. The orientation can be in values, directions, angles, terms, or any combination of these orientations. Orientations, for example, of terms or angles, can be approximate orientations. The orientations vary due to, for example, measurement or manufacturing tolerances or other factors. An orientation can vary, for example, by plus or minus 0.1 percent, plus or minus 0.2 percent, plus or minus 0.5 percent, plus or minus 1 percent, plus or minus 5 percent, plus or minus 10 percent, plus or minus 15 percent, or plus or minus 20 percent. Terms, such as about, substantially, approximately, or other relative terms can include the described ranges as will be readily understood by those of ordinary skill in the art and can include ranges that will be understood by those of ordinary skill in the art.

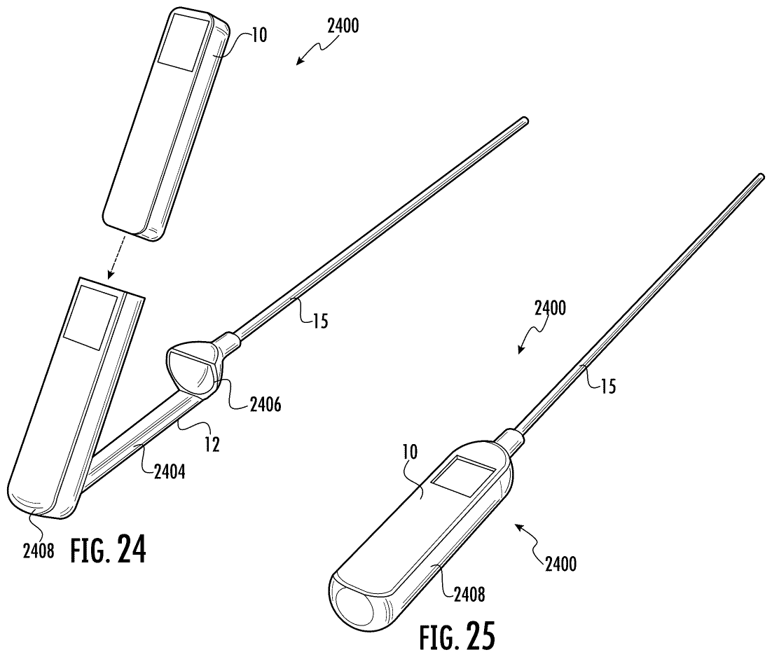

FIGS. 24-25 show a laparoscopic oximeter 2400, in an implementation. The oximeter includes a durable unit 10 and a measurement unit 12 (that can be referred to as a disposable unit). The measurement unit 12 includes a bar 2404 that extends from an end of the lid 2406 of the measurement unit to a base of the sheath 2408 of the measurement unit. The sheath 2408 is hinge-coupled, by a hinge, to the end of the bar 2404 where the bar connects to the sheath. The hinge has an axis of rotation that is transverse to a longitudinal axis of the housing and a longitudinal axis of the laparoscopic element. Compared to the implementation described above and shown in FIGS. 10A-11C, the hinge of the disposable unit is positioned closer to the proximal end of the device than the laparoscopic tube.

The sheath can be rotated about the hinge with respect to the bar and lid to connect the openings of the sheath and the lid to form a sealed space in the closed sheath and lid. More specifically, the lid rotates relative to the housing and the laparoscopic element of the measurement unit to form the sealed space and open the sealed space. The closed sheath and lid form a closed housing.

The sheath and lid include a latch that latches the sheath and lid closed when the sealed space is formed.

The durable unit 10 can be inserted into the sheath, and when the lid and sheath are rotated into contact, the durable unit is sealed in the sealed space. The durable unit and lid can include electrical contacts that contact when the lid and sheath are rotated to contact and form the sealed space. The durable unit and lid can include electrical contacts that contact when the lid and sheath are rotated to contact and form the sealed space.

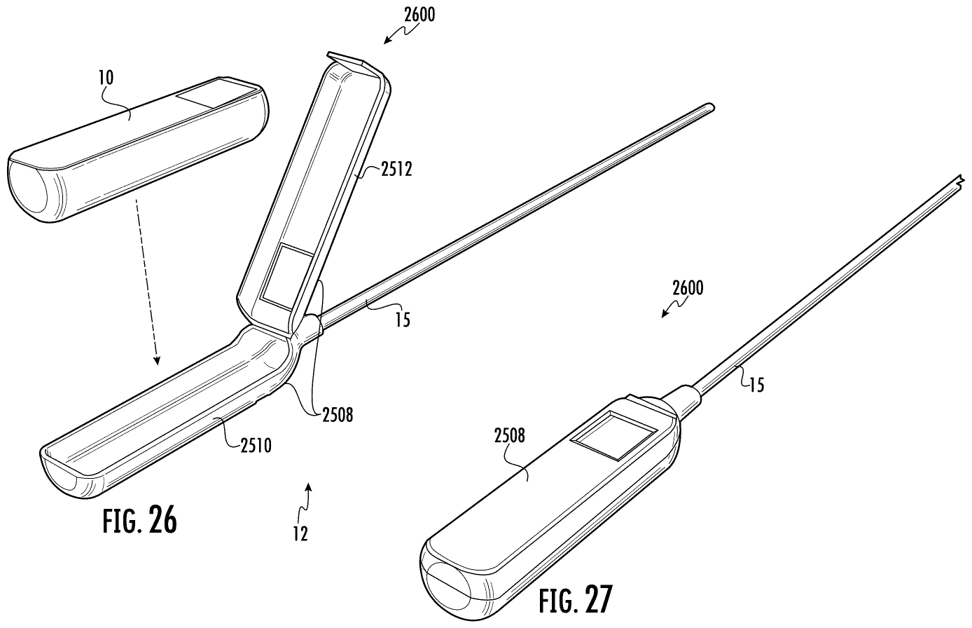

FIGS. 26-27 show a laparoscopic oximeter 2600, in an implementation. The oximeter includes a durable unit 10 and a measurement unit 12. The measurement unit includes a sheath 2508 that includes a housing 2510 with a lid 2512. At least a portion an outside edge of an opening of the housing is parallel to an axis passing through the laparoscopic tube, such as along a longitudinal axis of the tube.

The lid (which may be referred to as a sheath lid or an upper housing portion of the sheath) is connected, by a hinge, to the housing (which may be referred to as a sheath housing or a lower portion of the sheath) at an end of the housing and an end of the lid. More specifically, the hinge connects the lid at a distal end of the sheath to the housing. The hinge can include a hinge pin or the like with an axis of rotation is transverse or perpendicular to an axis of the laparoscopic tube, such as a longitudinal axis. The lid and housing portions of the sheath can be closed to enclose the processing unit. The hinge has an axis of rotation that is transverse to a longitudinal axis of the housing and a longitudinal axis of the laparoscopic element. The hinge of the measurement unit is positioned closer to the lid and laparoscopic tube than to the proximal end of the device. The lid rotates relative to the housing and the laparoscopic element of the measurement unit to form the sealed space and open the sealed space.

The sheath and lid include a latch that latches the sheath and lid closed when the sealed space is formed.

The durable unit 10 can be placed into the housing and the lid closed onto the housing to form a sealed area within the closed sheath. The durable unit is sealed in the sealed area when the lid is closed onto the housing to form the closed sheath. The durable unit and lid can include electrical contacts that contact when the lid and sheath are rotated to contact and form the sealed space.

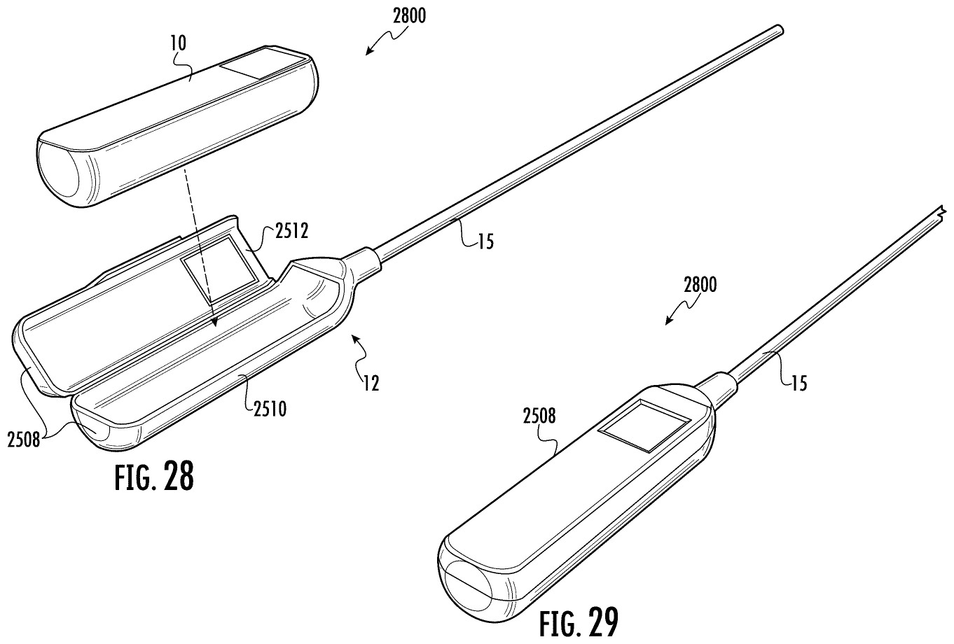

FIGS. 28-29 show a laparoscopic oximeter 2800, in an implementation. The oximeter includes a durable unit 10 and a measurement unit 12. The measurement unit includes a sheath 2508 that includes a housing 2510 with a lid 2512. The lid is hinge-coupled, by a hinge, to the housing at an end of the housing and an end of the lid. The hinge has an axis of rotation that is parallel to a longitudinal axis of the housing and a longitudinal axis of the laparoscopic element. The lid rotates relative to the housing and the laparoscopic element of the measurement unit to form the closed and sealed space and to open the closed and sealed space. The sheath and lid include a latch that latches the sheath and lid closed when the sealed space is formed.

At least a portion an outside edge of the opening of the housing is parallel to an axis passing through the laparoscopic tube, such as a longitudinal axis of the laparoscopic tube. However, in contrast to the laparoscopic oximeter 2600 shown in FIGS. 26-27, the hinge connecting the housing and lid of the sheath is along a longer edge of sheath. For example, in an implementation, a hinge pin or axis of rotation of the hinge is parallel to the axis of the laparoscopic tube, such as the longitudinal axis of the laparoscopic tube. The durable unit can be placed into the housing and the lid closed onto the housing to form the sealed area within the closed sheath. The durable unit is sealed in the sealed area when the lid is closed onto the housing to form the closed sheath.

For the implementations of FIGS. 24-29, the durable or processing unit described above and shown in FIGS. 14A-14C can be inserted and fit into the sheath or sheath housings. Also, the measurement unit (or disposable unit) of the implementations will have a connector and circuits that are compatible with the durable or processing unit. For example, these disposable implementations will include the connector and circuitry described above and shown in FIG. 13. Also, additional disposable unit implementations with, for example, different hinge positions, variations, and configurations are possible (not necessarily only those depicted—but possibly based on or variations of those depicted) that would be compatible with the durable or processing unit.

The sources, detectors, or both of the durable unit can be calibrated by one or more of a variety method. The following described steps may be used by the durable unit for calibrating the sources, detectors, or both. Steps may be added to, removed from, or combined in the method without deviating from the scope of the embodiment.

In an embodiment, sensor head 25 contacts a tissue phantom, which has homogeneous optical properties. Light (e.g., visible light, near-infrared light, or both) is emitted from one or more the light sources (e.g., S1) into the tissue phantom and at least some of the light is reflected back by the tissue phantom. Each detector receives a portion of the light reflected from the tissue phantom, and each detector generates reflectance data (i.e., a response) for the portion of reflected light received. The reflectance data for the detectors may not match a reflectance curve for the tissue phantom (i.e., may be offset from the reflectance curve). If the reflectance data generated by the detectors does not match the reflectance curve for the tissue phantom, the detectors may have an intrinsic gain or loss. The reflectance data generated is used by the oximeter or a separate computer system to generate a set of calibration functions so that the raw reflectance data matches the reflectance curve for the tissue phantom. Raw reflectance data includes the reflectance data generated and output by the detectors prior to being utilized for determining the optical properties for the tissue and before being utilized for determining oxygen saturation for the tissue.

The foregoing described steps may be repeated for one or more tissue phantoms where the different tissues phantoms have different optical properties, such as different reflection coefficients, different absorption coefficients, or both. The calibration function for each source-detector pair for each tissue phantom should generally be the same. However, if there is a deviation between the calibration functions for a given source-detector pair for a number of tissue phantoms, then the factors within the calibration function for the given source-detector might be averaged. Each of the calibration functions generated (including averaged functions) are stored in memory (e.g., Flash or other nonvolatile memory, or a programmable ROM).

The foregoing described steps may be repeated for each of the light sources, such as light sources S2, S3, or both. If the steps are repeated for each light source, then the calibration functions for these light sources may be stored in memory for each source-detector pair. That is, each source-detector pair has a calibration function specifically for the source-detector pair. For example, detector D1 a may have a first calibration functions stored for light emitted from light source S1 and a second calibration function for D1 for light emitted from light source S2. Because a calibration function can be stored for each source-detector pair, the calibration functions (e.g., two calibration functions) for each detector provide calibration not only for variations in the detectors but also for variations in the light sources. For example, the intrinsic gain or loss for a detector should not vary when receiving light from light source S1 or S2. If the two calibration functions differ for a detector when receiving reflected light for light source S1 a and thereafter for S2, the difference in the reflectance data for a given tissue phantom is attributable to differences in the intensity of light emitted by the light sources. The calibration functions can correct for these intensity differences.

The calibration functions may be applied to reflectance data that is generated by the detectors when the oximeter is used for oxygen saturation measurement in real tissue, for example, so that any intrinsic gains or losses of the detectors, and any difference in the intensity of light from light sources, may be compensated for. Specifically, the calibration functions are applied on a source-detector pair basis for the raw reflectance data generated by the detectors.

In an implementation, sensor head 25 includes a dispenser that can mark tissue. The laparoscopic tube can include a reservoir for dye or can be connected by a tube to a reservoir in the lid. The processor can control marking tissue. For example, marked tissue can indicate different ranges of oxygen saturation of tissue.

For measured tissue, the processor can determine an oxygen saturation value for the tissue based on reflectance data. Thereafter, the processor can determine a range of oxygen saturation from a plurality of ranges of oxygen saturation in which the oxygen saturation lies. The processor can control the tissue marker to mark the tissue with dye based on a range in which the oxygen saturation is in. For example, the processor may be configured to control the dispenser to mark the tissue with dye if the oxygen saturation is in a first range of oxygen saturation, but not mark the tissue if the oxygen saturation in a second range of oxygen saturation where the first range and second range are different, such as non-overlapping ranges. While the foregoing example embodiment discusses the utilization of two ranges of oxygen saturation by the oximeter, the oximeter may utilize more than two ranges of oxygen saturation for determining whether to mark the proved tissue with dye.

This description of the invention has been presented for the purposes of illustration and description. It is not intended to be exhaustive or to limit the invention to the precise form described, and many modifications and variations are possible in light of the teaching above. Elements of the various implementations can be used with other implementations in a number of ways, such as combinations, substitutions, or both. The implementations were chosen and described in order to best explain the principles of the invention and its practical applications. This description will enable others skilled in the art to best utilize and practice the invention in various implementations and with various modifications as are suited to a particular use. The scope of the invention is defined by the following claims.

What is claimed is:

1. A method comprising:
providing an upper portion of a sheath and a lower portion of the sheath;
coupling the upper and lower portions of the sheath with a hinge, such that the upper and lower portions can rotate relative to each other by the hinge;
providing a first opening in the lower portion at a distal end of the lower portion and a second opening in the lower portion at a proximal end of the lower portion;
providing a third opening in the upper portion at a distal end of the upper portion;
providing for, by rotation of the hinge, mating the third opening with the second opening to form a sealed enclosed space within the upper and lower portions;
coupling a laparoscopic tube the first opening of the lower portion at a proximal end of the laparoscopic tube; and
coupling sensor head to a distal end of the laparoscopic tube.

2. The method of claim 1 comprising positioning an electrical connector inside the lower portion of the sheath.

3. The method of claim 1 comprising providing a first latch portion on the lower portion of the sheath and a second latch portion on the upper portion of the sheath; and
when the third opening is mated with the second opening, providing for the first latch portion to latch to the second latch portion to hold the lower and upper portions together in a latched configuration to form the sealed enclosed space.

4. The method of claim 1 comprising providing the lower portion comprises a first polymer material; and
providing the upper portion comprises a second polymer material, different from the first polymer material.

5. The method of claim 4 comprising providing the first polymer material comprises an opaque material and the second polymer material comprises a translucent material.

6. The method of claim 1 comprising providing the lower portion of the sheath comprises a first polymer material and a second polymer material, and a first latch portion that comprises first and second cutaway sections;
providing omitting the first polymer material in the first and second cutaway sections;
providing the first and second cutaway sections are filled with the second polymer material; and
providing the second polymer material has a greater elasticity than the first polymer material.

7. The method of claim 1 comprising providing the lower portion of the sheath comprises a first polymer material and a second polymer material, and a button portion that comprises a cutaway section;
providing omitting the first polymer material in button portion;
providing the button portion is filled with the second polymer material; and
providing the second polymer material has a greater elasticity than the first polymer material.

8. The method of claim 1 comprising providing an electrical connector in the lower portion;
providing a printed circuit board in the lower portion that comprises electrical circuitry and is coupled to the electrical connector; and
providing a plurality of optical conductors coupled between the electrical circuitry and the sensor head.

9. The method of claim 8 wherein the electrical circuitry comprises light emitting diodes or laser diodes.

10. The method of claim 8 wherein the electrical circuitry comprises photodetectors.

11. The method of claim 1 comprising a printed circuit board, coupled to the lower portion of the sheath, wherein the printed circuit board comprises a first side and a second side, the first side of the printed circuit board comprises an electrical connector, and the second side of the printed circuit board comprises a first light emitting diode and a second light emitting diode, and a first photodetector and a second photodetector.

12. The method of claim 11 wherein the first and second light emitting diodes are formed on a first line, the first and second photodetector are formed on a second line, and the first and second lines do not intersect.

13. The method of claim 12 comprising a first fiber optic coupled between the first photodetector and a first detector structure of the sensor head.

14. The method of claim 13 comprising a second fiber optic coupled between the first light emitting diode and a second source structure of the sensor head.

15. The method of claim 1 comprising a first riser block mounted above the first and second light emitting diodes, wherein the first riser block comprises a first opening, a second opening, and a third opening, the first opening is in-line with an axis of the first emitting diode, the third opening is in-line with an axis of the second emitting diode, and the second opening is positioned between the first and third openings.

16. The method of claim 15 comprising a screw passing through the second opening and coupled to a screw receptacle formed in the printed circuit board.

17. A method comprising:
providing an enclosure for a portion of an oximeter device;
providing an interior space inside the enclosure;
providing an electrical connector at a distal end of the enclosure;
providing a printed circuit board in the interior space of the enclosure and electrical circuitry coupled to the printed circuit board and the electrical connector;
providing a battery compartment, formed in the enclosure;
covering the battery compartment with a battery cover that forms an exterior surface of the enclosure; and
providing a display coupled to the enclosure and the printed circuit board.

18. The method of claim 17 wherein the electrical circuitry comprises a processor and wireless communication circuit.

19. The method of claim 18 comprising displaying in a first screen of the display a numerical percentage of oxygen saturation value.

20. The method of claim 19 comprising displaying in the first screen of the display a battery level indication.

21. The method of claim 19 comprising displaying in the first screen of the display a bar level indication of the oxygen saturation value.

22. The method of claim 17 comprising providing the portion of the oximeter device determines an oxygen saturation value, and the portion of the oximeter device does not include light emitting diodes or laser diodes or photodetectors.

23. The method of claim 22 comprising providing for data optical measurement information to transfer to the portion via the electrical connector.

24. The method of claim 21 comprising providing for data optical measurement information to transfer to the portion via a wireless communication circuit of the electrical circuitry.

25. A method comprising:
providing a first device portion comprising:
providing an upper portion of a sheath of the first device portion and a lower portion of the sheath of the first device portion;
coupling the upper and lower portions of the sheath with a hinge, such that the upper and lower portions can rotate relative to each other by the hinge;
providing a first opening in the lower portion at a distal end of the lower portion and a second opening in the lower portion at a proximal end of the lower portion;
providing a third opening in the upper portion at a distal end of the upper portion;
providing for, by rotation of the hinge, mating the third opening with the second opening to form a sealed enclosed space within the upper and lower portions;
coupling a laparoscopic tube the first opening of the lower portion at a proximal end of the laparoscopic tube; and
coupling sensor head to a distal end of the laparoscopic tube providing a second device portion comprising:
providing an enclosure for the second device portion;
providing an interior space inside the enclosure;
providing an electrical connector at a distal end of the enclosure;
providing a printed circuit board in the interior space of the enclosure and electrical circuitry coupled to the printed circuit board and the electrical connector;
providing a battery compartment, formed in the enclosure;
covering the battery compartment with a battery cover that forms an exterior surface of the enclosure; and
providing a display coupled to the enclosure and the printed circuit board, wherein the enclosure of the second device portion fits into the upper portion of the sheath of the first device portion, and when the third opening mates with the second opening, the enclosure of the second device portion is within the sealed enclosed space within the upper and lower portions of the first device portion.

26. The device of claim 25 comprising providing the lower portion comprises an electrical connector, and when the enclosure is within the sealed enclosed space, the electrical connector of the enclosure couples to the electrical connector of the lower portion.

27. The method of claim 26 comprising providing for data optical measurement information to transfer from the lower portion to the enclosure through the electrical connectors of the lower portion and the enclosure in a digital format.

28. The method of claim 27 comprising providing for data optical measurement information to transfer from the lower portion to the enclosure through the electrical connectors of the lower portion and the enclosure in a digital format an not an analog format.

29. The method of claim 25 wherein the electrical circuitry comprises a processor and wireless communication circuit.

30. The method of claim 29 comprising providing the processor determines an oxygen saturation value, and the enclosure does not include light emitting diodes or laser diodes or photodetectors.

31. The method of claim 29 comprising displaying in a first screen of the display a numerical percentage of oxygen saturation value.

32. The method of claim 29 comprising displaying in the first screen of the display a battery level indication.

33. The method of claim 29 comprising displaying in the first screen of the display a bar level indication of the oxygen saturation value.
